SEQUENCE LISTING (72 sequences)

SEQ ID NO: 1 - protein, 177 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 2 - protein, 154 aa, Rattus norvegicus
MFHVSFRYIFGIPPLILVLLPVTSSDCHIKDKDGKAFGSVLMISINQLDKMTGTDSDCPNNEPNFFKKHLCDDTKEAAFLNRAARKLRQFLKMNISEEFNDHLLRVSDGTQTLVNCTSKEEKTIKEQKKNDPCFLKRLLREIKTCWNKILKGSI

SEQ ID NO: 3 - protein, 154 aa, Mus Musculus
MFHVSFRYIFGIPPLILVLLPVTSSECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSI

SEQ ID NO: 4 - protein, 177 aa, Cercopithecus aethiops
MFHVSFRYIFGLPPLILVLLPVASSDCDIEGKDGKQYESVLMVSIDQLLDSMKEIGSNCLNNEFNFFKRHLCDDNKEGMFLFRAARKLKQFLKMNSTGDFDLHLLKVSEGTTILLNCTGKVKGRKPAALGEPQPTKSLEENKSLKEQKKLNDSCFLKRLLQKIKTCWNKILMGTKEH

SEQ ID NO: 5 - protein, 176 aa, Bos taurus
MFHVSFRYIFGIPPLILVLLPVASSDCDISGKDGGAYQNVLMVNIDDLDNMINFDSNCLNNEPNFFKKHSCDDNKEASFLNRASRKLRQFLKMNISDDFKLHLSTVSQGTLTLLNCTSKGKGRKPPSLSEAQPTKNLEENKSSKEQKKQNDLCFLKILLQKIKTCWNKILRGIKEH

SEQ ID NO: 6 - protein, 176 aa, Ovis aries
MFHVSFRYIFGIPPLILVLLPVASSDCDFSGKDGGAYQNVLMVSIDDLDNMINFDSNCLNNEPNFFKKHSCDDNKEASFLNRAARKLKQFLKMNISDDFKLHLSTVSQGTLTLLNCTSKGKGRKPPSLGEAQPTKNLEENKSLKEQRKQNDLCFLKILLQKIKTCWNKILRGITEH

SEQ ID NO: 7 - protein, 384 aa, Artificial Sequence
  amino acid sequence of human IgD constant region (Genbank accession No. P01880)
APTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDHGPMK

SEQ ID NO: 8 - protein, 327 aa, Artificial Sequence
  amino acid sequence of Partial human IgG4 constant region (Genbank accession No. AAH25985)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 9 - protein, 245 aa, Artificial Sequence
  amino acid sequence of hyFc
RNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 245 aa, Artificial Sequence
  amino acid sequence of hyFcM1
RNTGRGGEEKKGGKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 245 aa, Artificial Sequence
  amino acid sequence of hyFcM2
RNTGRGGEEKKGSKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 245 aa, Artificial Sequence
  amino acid sequence of hyFcM3
RNTGRGGEEKKSGKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 245 aa, Artificial Sequence
  amino acid sequence of hyFcM4
RNTGRGGEEKKSSKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 243 aa, Artificial Sequence
  amino acid sequence of mouse IgG Fc variant
ASAEPRGPTIKPCPPCKCPAPNLEGGPSVFIFPPKIKDVLMISLSPIVTCVVVDVSEDDPDVQISWFVNNVEVHTAQTQTHREDYNSTLRVVSALPIQHQDWMSGKAFACAVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGNSGS

SEQ ID NO: 15 - protein, 153 aa, Artificial Sequence
  amino acid sequence of modified IL-7(M)
MDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 16 - protein, 154 aa, Artificial Sequence
  amino acid sequence of modified IL-7(MM)
MMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 17 - protein, 155 aa, Artificial Sequence
  amino acid sequence of modified IL-7(MMM)
MMMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 18 - protein, 155 aa, Artificial Sequence
  amino acid sequence of modified IL-7(MGM)
MGMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 19 - protein, 155 aa, Artificial Sequence
  amino acid sequence of modified IL-7(DDD)
DDDDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 20 - protein, 156 aa, Artificial Sequence
  amino acid sequence of modified IL-7(MMMM)
MMMMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 21 - protein, 398 aa, Artificial Sequence
  amino acid sequence of modified IL-7(M) fused hyFc
MDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 399 aa, Artificial Sequence
  amino acid sequence of modified IL-7(MM) fused hyFc
MMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 23 - protein, 400 aa, Artificial Sequence
  amino acid sequence of modified IL-7(MMM) fused hyFc
MMMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 24 - protein, 400 aa, Artificial Sequence
  amino acid sequence of modified IL-7(MGM) fused hyFc
MGMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 25 - protein, 401 aa, Artificial Sequence
  amino acid sequence of modified IL-7(MMMM) fused hyFc
MMMMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 26 - protein, 397 aa, Artificial Sequence
  amino acid sequence of human IL-7 fused hyFc
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 395 aa, Artificial Sequence
  amino acid sequence of human IL-7 fused nonlytic mouse Fc
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHASAEPRGPTIKPCPPCKCPAPNLEGGPSVFIFPPKIKDVLMISLSPIVTCVVVDVSEDDPDVQISWFVNNVEVHTAQTQTHREDYNSTLRVVSALPIQHQDWMSGKAFACAVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGNSGS

SEQ ID NO: 28 - DNA, 531 bp, Artificial Sequence
  nucleotide sequence of human IL-7
atgttccacgtgagcttcaggtacatcttcggcctgccacccctgatcctggtgctgctgcctgtggccagctccgactgcgacatcgagggaaaagacggcaagcagtacgaaagcgtgctgatggtgtccatcgaccagctgctggattctatgaaggagattgggagtaactgcctgaacaatgagttcaacttcttcaaacggcacatttgtgatgccaacaaggagggaatgttcctgtttcgggccgctagaaaactgaggcagttcctgaagatgaacagcaccggagactttgatctgcatctgctgaaagtgtctgagggcaccacaatcctgctgaactgcactgggcaggtgaaaggaaggaagcctgccgctctgggagaggctcagccaaccaagtcactggaggaaaacaaaagcctgaaggaacagaagaaactgaatgacctgtgctttctgaaacggctgctgcaggagatcaaaacatgttggaacaagattctgatgggcacaaaggaacac

SEQ ID NO: 29 - DNA, 534 bp, Artificial Sequence
  nucleotide sequence of modified IL-7(M)
atgttccacgtgagcttcagatacatcttcggcctgccccccctgatcctggtgctgctgcccgtggccagcagcatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcac

SEQ ID NO: 30 - DNA, 537 bp, Artificial Sequence
  nucleotide sequence of modified IL-7(MM)
atgttccacgtgagcttcagatacatcttcggcctgccccccctgatcctggtgctgctgcccgtggccagcagcatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcac

SEQ ID NO: 31 - DNA, 540 bp, Artificial Sequence
  nucleotide sequence of modified IL-7(MMM)
atgttccacgtgagcttcagatacatcttcggcctgccccccctgatcctggtgctgctgcccgtggccagcagcatgatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcac

SEQ ID NO: 32 - DNA, 540 bp, Artificial Sequence
  nucleotide sequence of modified IL-7(MGM)
atgttccacgtgagcttcaggtacatcttcggcctgccacccctgatcctggtgctgctgcctgtggccagctccatggggatggactgcgacatcgagggaaaagacggcaagcagtacgaaagcgtgctgatggtgtccatcgaccagctgctggattctatgaaggagattgggagtaactgcctgaacaatgagttcaacttcttcaaacggcacatttgtgatgccaacaaggagggaatgttcctgtttcgggccgctagaaaactgaggcagttcctgaagatgaacagcaccggagactttgatctgcatctgctgaaagtgtctgagggcaccacaatcctgctgaactgcactgggcaggtgaaaggaaggaagcctgccgctctgggagaggctcagccaaccaagtcactggaggaaaacaaaagcctgaaggaacagaagaaactgaatgacctgtgctttctgaaacggctgctgcaggagatcaaaacatgttggaacaagattctgatgggcaccaaggagcac

SEQ ID NO: 33 - DNA, 540 bp, Artificial Sequence
  nucleotide sequence of modified IL-7(DDD)
atgttccacgtgagcttcagatacatcttcggcctgccccccctgatcctggtgctgctgcccgtggccagcagcgacgatgacgactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcac

SEQ ID NO: 34 - DNA, 543 bp, Artificial Sequence
  nucleotide sequence of modified IL-7(MMMM)
atgttccacgtgagcttcagatacatcttcggcctgccccccctgatcctggtgctgctgcccgtggccagcagcatgatgatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcac

SEQ ID NO: 35 - DNA, 1284 bp, Artificial Sequence
  nucleotide sequence of modified IL-7(M) fused hyFc
atgttccacgtgagcttcagatacatcttcggcctgccccccctgatcctggtgctgctgcccgtggccagcagcatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcacaggaacacaggcagaggcggcgaggagaagaagaaggagaaggagaaggaggagcaggaggaaagagagaccaagacccccgagtgccccagccacacccagcccctgggcgtgttcctgttccctcccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaagtgactcgagtctaga

SEQ ID NO: 36 - DNA, 1272 bp, Artificial Sequence
  nucleotide sequence of modified IL-7(MM) fused hyFc
atgttccacgtgagcttcagatacatcttcggcctgccccccctgatcctggtgctgctgcccgtggccagcagcatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcacaggaacacaggcagaggcggcgaggagaagaagaaggagaaggagaaggaggagcaggaggaaagagagaccaagacccccgagtgccccagccacacccagcccctgggcgtgttcctgttccctcccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 37 - DNA, 1275 bp, Artificial Sequence
  nucleotide sequence of modified IL-7(MMM) fused hyFc
atgttccacgtgagcttcagatacatcttcggcctgccccccctgatcctggtgctgctgcccgtggccagcagcatgatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcacaggaacacaggcagaggcggcgaggagaagaagaaggagaaggagaaggaggagcaggaggaaagagagaccaagacccccgagtgccccagccacacccagcccctgggcgtgttcctgttccctcccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 38 - DNA, 1275 bp, Artificial Sequence
  nucleotide sequence of modified IL-7(MGM) fused hyFc
atgttccacgtgagcttcaggtacatcttcggcctgccacccctgatcctggtgctgctgcctgtggccagctccatggggatggactgcgacatcgagggaaaagacggcaagcagtacgaaagcgtgctgatggtgtccatcgaccagctgctggattctatgaaggagattgggagtaactgcctgaacaatgagttcaacttcttcaaacggcacatttgtgatgccaacaaggagggaatgttcctgtttcgggccgctagaaaactgaggcagttcctgaagatgaacagcaccggagactttgatctgcatctgctgaaagtgtctgagggcaccacaatcctgctgaactgcactgggcaggtgaaaggaaggaagcctgccgctctgggagaggctcagccaaccaagtcactggaggaaaacaaaagcctgaaggaacagaagaaactgaatgacctgtgctttctgaaacggctgctgcaggagatcaaaacatgttggaacaagattctgatgggcacaaaggaacaccgcaatactgggcggggcggggaggaaaagaaaaaggagaaggaaaaggaggaacaggaggaaagagagactaagaccccagaatgtcccagccatactcagcccctgggggtgttcctgtttccccctaaacctaaggataccctgatgatcagcaggacacccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 39 - DNA, 1278 bp, Artificial Sequence
  nucleotide sequence of modified IL-7(MMMM) fused hyFc
atgttccacgtgagcttcagatacatcttcggcctgccccccctgatcctggtgctgctgcccgtggccagcagcatgatgatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcacaggaacacaggcagaggcggcgaggagaagaagaaggagaaggagaaggaggagcaggaggaaagagagaccaagacccccgagtgccccagccacacccagcccctgggcgtgttcctgttccctcccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 40 - protein, 4 aa, Artificial Sequence
  synthetic oligopeptide fragment
MMMM

SEQ ID NO: 41 - protein, 4 aa, Artificial Sequence
  synthetic oligopeptide fragment
MGGM

SEQ ID NO: 42 - protein, 4 aa, Artificial Sequence
  synthetic oligopeptide fragment
MMGG

SEQ ID NO: 43 - protein, 4 aa, Artificial Sequence
  synthetic oligopeptide fragment
GGMM

SEQ ID NO: 44 - protein, 4 aa, Artificial Sequence
  synthetic oligopeptide fragment
MGMG

SEQ ID NO: 45 - protein, 4 aa, Artificial Sequence
  synthetic oligopeptide fragment
GMMG

SEQ ID NO: 46 - protein, 4 aa, Artificial Sequence
  synthetic oligopeptide fragment
GGGM

SEQ ID NO: 47 - protein, 4 aa, Artificial Sequence
  synthetic oligopeptide fragment
MGGG

SEQ ID NO: 48 - protein, 4 aa, Artificial Sequence
  synthetic oligopeptide fragment
GMGG

SEQ ID NO: 49 - protein, 4 aa, Artificial Sequence
  synthetic oligopeptide fragment
GGMG

SEQ ID NO: 50 - protein, 5 aa, Artificial Sequence
  synthetic oligopeptide fragment
GGMMM

SEQ ID NO: 51 - protein, 5 aa, Artificial Sequence
  synthetic oligopeptide fragment
GGGMM

SEQ ID NO: 52 - protein, 5 aa, Artificial Sequence
  synthetic oligopeptide fragment
GGGGM

SEQ ID NO: 53 - protein, 5 aa, Artificial Sequence
  synthetic oligopeptide fragment
MGMMM

SEQ ID NO: 54 - protein, 5 aa, Artificial Sequence
  synthetic oligopeptide fragment
MGGMM

SEQ ID NO: 55 - protein, 5 aa, Artificial Sequence
  synthetic oligopeptide fragment
MGGGM

SEQ ID NO: 56 - protein, 5 aa, Artificial Sequence
  synthetic oligopeptide fragment
MMGMM

SEQ ID NO: 57 - protein, 5 aa, Artificial Sequence
  synthetic oligopeptide fragment
MMGGM

SEQ ID NO: 58 - protein, 5 aa, Artificial Sequence
  synthetic oligopeptide fragment
MMGGG

SEQ ID NO: 59 - protein, 5 aa, Artificial Sequence
  synthetic oligopeptide fragment
MMMGM

SEQ ID NO: 60 - protein, 5 aa, Artificial Sequence
  synthetic oligopeptide fragment
MGMGM

SEQ ID NO: 61 - protein, 5 aa, Artificial Sequence
  synthetic oligopeptide fragment
GMGMG

SEQ ID NO: 62 - protein, 5 aa, Artificial Sequence
  synthetic oligopeptide fragment
GMMMG

SEQ ID NO: 63 - protein, 5 aa, Artificial Sequence
  synthetic oligopeptide fragment
GGMGM

SEQ ID NO: 64 - protein, 5 aa, Artificial Sequence
  synthetic oligopeptide fragment
GGMMG

SEQ ID NO: 65 - protein, 5 aa, Artificial Sequence
  synthetic oligopeptide fragment
GMMGM

SEQ ID NO: 66 - protein, 5 aa, Artificial Sequence
  synthetic oligopeptide fragment
MGMMG

SEQ ID NO: 67 - protein, 5 aa, Artificial Sequence
  synthetic oligopeptide fragment
GMGGM

SEQ ID NO: 68 - protein, 5 aa, Artificial Sequence
  synthetic oligopeptide fragment
MMGMG

SEQ ID NO: 69 - protein, 5 aa, Artificial Sequence
  synthetic oligopeptide fragment
GMMGG

SEQ ID NO: 70 - protein, 5 aa, Artificial Sequence
  synthetic oligopeptide fragment
GMGGG

SEQ ID NO: 71 - protein, 5 aa, Artificial Sequence
  synthetic oligopeptide fragment
GGMGG

SEQ ID NO: 72 - protein, 5 aa, Artificial Sequence
  synthetic oligopeptide fragment
GGGGG

(19) United States
(12) Patent Application Publication
(10) Pub. No.: US 2023/0210952 A1
(43) Pub. Date: Jul. 6, 2023

(54) METHOD OF TREATING A SOLID TUMOR WITH A COMBINATION OF AN IL-7 PROTEIN AND CAR-BEARING IMMUNE CELLS

(71) Applicants: Washington University, St. Louis, MO (US); NeoImmuneTech, Inc., Rockville, MD (US)

(72) Inventors: John F. DIPERSIO, St. Louis, MO (US); Matthew COOPER, St. Louis, MO (US); Se Hwan YANG, Seongnam-si (KR); Byung Ha LEE, Rockville, MD (US); Donghoon CHOI, Seongnam-si (KR)

(73) Assignees: Washington University, St. Louis, MO (US); NeoImmuneTech, Inc., Rockville, MD (US)

(21) Appl. No.: 17/760,290

(22) PCT Filed: Feb. 4, 2021
(86) PCT No.: PCT/US2021/016604

§ 371 Date: Aug. 5, 2022

Related U.S. Application Data
(60) Provisional application No. 62/970,554, filed on Feb. 5, 2020.

(51) Int. Cl. A61K 38/20; A61K 35/17; A61P 35/00

(52) U.S. Cl. CPC A61K 38/2046; A61K 35/17; A61P 35/00

(57) ABSTRACT

The present disclosure relates to methods of treating a solid tumor with an IL-7 protein in combination with a population of modified immune cells, e.g, CAR-bearing immune cells (e.g., CAR-T cells or iNKT-CAR cells) and/or transgenic TCR-bearing immune cells.

### CROSS-REFERENCE TO RELATED APPLICATIONS

This PCT application claims the priority benefit of U.S. Provisional Application No. 62/970,554, filed Feb. 5, 2020, which is incorporated herein by reference in its entirety.

### REFERENCE TO SEQUENCE LISTING SUBMITTED ELECTRONICALLY VIA EFS-WEB

The content of the electronically submitted sequence listing in ASCII text file (Name: 4241_014PC01_SequenceListing_ST25.txt; Size: 83,475 bytes; and Date of Creation: Feb. 3, 2021) filed with the application is herein incorporated by reference in its entirety.

### BACKGROUND OF THE DISCLOSURE

Chimeric antigen receptor T cell (CAR-T) immunotherapy is increasingly well known. T cells are genetically modified to express chimeric antigen receptors (CARs), which are fusion proteins comprised of an antigen recognition moiety and T cell activation domains. The CARs are designed to recognize antigens that are overexpressed on cancer cells. CAR-Ts demonstrate exceptional clinical efficacy against B cell malignancies, and two therapies, KYMRIAH™ (tisagenlecleucel, Novartis) and YESCARTA™ (axicabtagene ciloleucel, Kite/Gilead) has been approved by the FDA. Recent disclosures have also shown promise in expanding the react of CAR-T therapy to T-cell malignancies as well, and in enabling “off-the-shelf” use of pre-engineered cells from donors to treat malignancies without allogenic reactivity.

However, challenges remain. CAR-T immunotherapy is limited by the successful expansion of engineered cells in a recipient's body; typically, a large infusion of cells is required. Additionally, loss of persistence of CAR-T cells infused into a subject have been observed, leading to loss of clinical efficacy and potential relapse. And to date, CAR-T therapy has been limited to hematologic malignancies due to the tumor microenvironment preventing access by tumor-infiltrating lymphocytes, including engineered cells. Therefore, there remains a need for new treatment options with acceptable safety profile and high efficacy in cancer patients, particularly those with solid tumors.

### SUMMARY OF THE DISCLOSURE

Provided herein is a method of treating a solid tumor in a subject in need thereof, comprising administering to the subject a population of immune cells comprising a chimeric antigen receptor (CAR) (“CAR-bearing immune cells”) in combination with an IL-7 protein.

In some aspects, a tumor volume is reduced in the subject after the administration compared to a reference tumor volume (e.g., tumor volume in a corresponding subject that received either IL-7 protein alone or CAR-bearing immune cells alone). In certain aspects, the tumor volume is reduced by at least about 5%, at least about 10%, at least about 20%, at least about 30%, at least about 40%, at least about 50%, at least about 60%, at least about 70%, at least about 80%, at least about 90%, or about 100% after the administration compared to the reference tumor volume.

In some aspects, a duration of survival of the subject is increased after the administration compared to a reference duration of survival (e.g., duration of survival in a corresponding subject that received either IL-7 protein alone or CAR-bearing immune cells alone). In certain aspects, the duration of survival is increased by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200% or more after the administration compared to the reference duration of survival.

Also disclosed herein is a method of enhancing an anti-tumor activity of immune cells comprising a chimeric antigen receptor (CAR) (“CAR-bearing immune cells”) in a subject having a solid tumor, comprising administering to the subject a population of the CAR-bearing immune cells in combination with an IL-7 protein.

In some aspects, the anti-tumor activity comprises a reduction in tumor volume, an increase in duration of survival, or both. In some aspects, the tumor volume is reduced by at least about 5%, at least about 10%, at least about 20%, at least about 30%, at least about 40%, at least about 50%, at least about 60%, at least about 70%, at least about 80%, at least about 90%, or about 100% after the administration compared to a reference tumor volume (e.g., tumor volume in a corresponding subject that received either IL-7 protein alone or CAR-bearing immune cells alone). In some aspects, the duration of survival is increased by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200% or more after the administration compared to a reference duration of survival (e.g., duration of survival in a corresponding subject that received either IL-7 protein alone or CAR-bearing immune cells alone).

Present disclosure further provides a method of increasing an expansion of immune cells comprising a chimeric antigen receptor (CAR) (“CAR-bearing immune cells”) in a subject having a solid tumor, comprising administering to the subject a population of the CAR-bearing immune cells in combination with an IL-7 protein. In some aspects, the expansion of the CAR-bearing immune cells is increased by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200% or more after the administration compared to a reference expansion (e.g., expansion of CAR-bearing immune cells in a corresponding subject that only received an administration of the population of CAR-bearing immune cells).

Provided herein is a method of increasing a survival and/or persistence of immune cells comprising a chimeric antigen receptor (CAR) (“CAR-bearing immune cells”) in a subject having a solid tumor, comprising administering to the subject a population of the CAR-bearing immune cells in combination with an IL-7 protein. In certain aspects, the survival and/or persistence of the CAR-bearing immune cells is increased by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200% or more after the administration compared to a reference survival and/or persistence (e.g., survival and/or persistence of CAR-bearing immune cells in a corresponding subject that only received an administration of the population of CAR-bearing immune cells).

In some aspects, methods disclosed herein comprises administering an IL-7 protein at a dose of greater than about 600 μg/kg, greater than about 700 μg/kg, greater than about 800 μg/kg, greater than about 900 μg/kg, greater than about 1,000 μg/kg, greater than about 1,100 μg/kg, greater than about 1,200 μg/kg, greater than about 1,300 μg/kg, greater than about 1,400 μg/kg, greater than about 1,500 μg/kg, greater than about 1,600 μg/kg, greater than about 1,700 μg/kg, greater than about 1,800 μg/kg, greater than about 1,900 μg/kg, or greater than about 2,000 μg/kg.

In some aspects, the IL-7 protein is administered at a dose of between about 610 μg/kg and about 1,200 μg/kg, between about 650 μg/kg and about 1,200 μg/kg, between about 700 μg/kg and about 1,200 μg/kg, between about 750 μg/kg and about 1,200 μg/kg, between about 800 μg/kg and about 1,200 μg/kg, between about 850 μg/kg and about 1,200 μg/kg, between about 900 μg/kg and about 1,200 μg/kg, between about 950 μg/kg and about 1,200 μg/kg, between about 1,000 μg/kg and about 1,200 μg/kg, between about 1,050 μg/kg and about 1,200 μg/kg, between about 1,100 μg/kg and about 1,200 μg/kg, between about 1,200 μg/kg and about 2,000 μg/kg, between about 1,300 μg/kg and about 2,000 μg/kg, between about 1,500 μg/kg and about 2,000 μg/kg, between about 1,700 μg/kg and about 2,000 μg/kg, between about 610 μg/kg and about 1,000 μg/kg, between about 650 μg/kg and about 1,000 μg/kg, between about 700 μg/kg and about 1,000 μg/kg, between about 750 μg/kg and about 1,000 μg/kg, between about 800 μg/kg and about 1,000 μg/kg, between about 850 μg/kg and about 1,000 μg/kg, between about 900 μg/kg and about 1,000 μg/kg, or between about 950 μg/kg and about 1,000 μg/kg.

In some aspects, the IL-7 protein is administered at a dose of between about 700 μg/kg and about 900 μg/kg, between about 750 μg/kg and about 950 μg/kg, between about 700 μg/kg and about 850 μg/kg, between about 750 μg/kg and about 850 μg/kg, between about 700 μg/kg and about 800 μg/kg, between about 800 μg/kg and about 900 μg/kg, between about 750 μg/kg and about 850 μg/kg, or between about 850 μg/kg and about 950 μg/kg.

In some aspects, the IL-7 protein is administered at a dose of about 650 μg/kg, about 680 μg/kg, about 700 μg/kg, about 720 μg/kg, about 740 μg/kg, about 750 μg/kg, about 760 μg/kg, about 780 μg/kg, about 800 μg/kg, about 820 μg/kg, about 840 μg/kg, about 850 μg/kg, about 860 μg/kg, about 880 μg/kg, about 900 μg/kg, about 920 μg/kg, about 940 μg/kg, about 950 μg/kg, about 960 μg/kg, about 980 μg/kg, about 1000 μg/kg, about 1100 μg/kg, about 1200 μg/kg, about 1,300 μg/kg, about 1,400 μg/kg, about 1,440 μg/kg, about 1,500 μg/kg, about 1,600 μg/kg, about 1,700 μg/kg, about 1,800 μg/kg, about 1,900 μg/kg, or about 2,000 μg/kg.

In some aspects, the IL-7 protein is administered at a dosing frequency of once a week, once in two weeks, once in three weeks, once in four weeks, once in five weeks, once in six weeks, once in seven weeks, once in eight weeks, once in nine weeks, once in 10 weeks, once in 11 weeks, or once in 12 weeks.

In some aspects, the methods disclosed herein comprises administering a population of CAR-bearing immune cells at a dose of less than about 100,000 CAR-bearing immune cells per kilogram of the subject's body weight. In some aspects, the population of CAR-bearing immune cells is administered at a dose of less than about 50,000 CAR-bearing immune cells per kilogram of the subject's body weight. In certain aspects, the population of CAR-bearing immune cells is administered at a dose of less than about 10,000 CAR-bearing immune cells per kilogram of the subject's body weight. In some aspects, the population of CAR-bearing immune cells is administered at a dose of less than about 5,000 CAR-bearing immune cells per kilogram of the subject's body weight. In some aspects, the population of CAR-bearing immune cells is administered at a dose of less than about 2,500 CAR-bearing immune cells per kilogram of the subject's body weight. In some aspects, the population of CAR-bearing immune cells is administered at a dose of less than about 1,000 CAR-bearing immune cells per kilogram of the subject's body weight.

In some aspects, the IL-7 protein and the population of CAR-bearing immune cells are administered concurrently. In some aspects, the IL-7 protein and the population of CAR-bearing immune cells are administered sequentially. In certain aspects, the IL-7 protein is administered to the subject prior to administering the population of CAR-bearing immune cells. In some aspects, the IL-7 protein is administered to the subject after administering the population of CAR-bearing immune cells.

In some aspects, the IL-7 protein is administered to the subject parenthetically, intramuscularly, subcutaneously, ophthalmic, intravenously, intraperitoneally, intradermally, intraorbitally, intracerebrally, intracranially, intraspinally, intraventricular, intrathecally, intracistemally, intracapsularly, or intratumorally. In some aspects, the population of CAR-bearing immune cells is administered intravenously.

In some aspects, an IL-7 protein that can be used in any of the methods disclosed herein is not a wild-type IL-7 protein (i.e., has been modified). In certain aspects, the IL-7 protein comprises an oligopeptide consisting of 1 to 10 amino acid residues. In some aspects, the oligopeptide comprises methionine (M), glycine (G), methionine-methionine (MM), glycine-glycine (GG), methionine-glycine (MG), glycine-methionine (GM), methionine-methionine-methionine (MMM), methionine-methionine-glycine (MMG), methionine-glycine-methionine (MGM), glycine-methionine-methionine (GMM), methionine-glycine-glycine (MGG), glycine-methionine-glycine (GMG), glycine-glycine-methionine (GGM), glycine-glycine-glycine (GGG), methionine-glycine-glycine-methionine (MGGM) (SEQ ID NO: 41), methionine-methionine-glycine-glycine (MMGG) (SEQ ID NO: 42), glycine-glycine-methionine-methionine (GGMM) (SEQ ID NO: 43), methionine-glycine-methionine-glycine (MGMG) (SEQ ID NO: 44), glycine-methionine-methionine-glycine (GMMG) (SEQ ID NO: 45), glycine-glycine-glycine-methionine (GGGM) (SEQ ID NO: 46), methionine-glycine-glycine-glycine (MGGG) (SEQ ID NO: 47), glycine-methionine-glycine-glycine (GMGG) (SEQ ID NO: 48), glycine-glycine-methionine-glycine (GGMG) (SEQ ID NO: 49), glycine-glycine-methionine-methionine-methionine (GGMMM) (SEQ ID NO: 50), glycine-glycine-glycine-methionine-methionine (GGGMM) (SEQ ID NO: 51), glycine-glycine-glycine-glycine-methionine (GGGGM) (SEQ ID NO: 52), methionine-glycine-methionine-methionine-methionine (MGMMM) (SEQ ID NO: 53), methionine-glycine-glycine-methionine-methionine (MGGMM) (SEQ ID NO: 54), methionine-glycine-glycine-glycine-methionine (MGGGM) (SEQ ID NO: 55), methionine-methionine-glycine-methionine-methionine (MMGMM) (SEQ ID NO: 56), methionine-methionine-glycine-glycine-methionine (MMGGM) (SEQ ID NO: 57), methionine-methionine-glycine-glycine-glycine (MMGGG) (SEQ ID NO: 58), methionine-methionine-methionine-glycine-methionine (MMMGM) (SEQ ID NO: 59), methionine-glycine-methionine-glycine-methionine (MGMGM) (SEQ ID NO: 60), glycine-methionine-glycine-methionine-glycine (GMGMG) (SEQ ID NO: 61), glycine-methionine-methionine-methionine-glycine (GMMMG) (SEQ ID NO: 62), glycine-glycine-methionine-glycine-methionine (GGMGM) (SEQ ID NO: 63), glycine-glycine-methionine-methionine-glycine (GGMMG) (SEQ ID NO: 64), glycine-methionine-methionine-glycine-methionine (GMMGM) (SEQ ID NO: 65), methionine-glycine-methionine-methionine-glycine (MGMMG) (SEQ ID NO: 66), glycine-methionine-glycine-glycine-methionine (GMGGM) (SEQ ID NO: 67), methionine-methionine-glycine-methionine-glycine (MMGMG) (SEQ ID NO: 68), glycine-methionine-methionine-glycine-glycine (GMMGG) (SEQ ID NO: 69), glycine-methionine-glycine-glycine-glycine (GMGGG) (SEQ ID NO: 70), glycine-glycine-methionine-glycine-glycine (GGMGG) (SEQ ID NO: 71), glycine-glycine-glycine-glycine-glycine (GGGGG) (SEQ ID NO: 72), or combinations thereof. In some aspects, the oligopeptide is methionine-glycine-methionine (MGM).

In some aspects, the IL-7 protein comprises a half-life extending moiety. In certain aspects, the half-life extending moiety comprises an Fc, albumin, an albumin-binding polypeptide, Pro/Ala/Ser (PAS), a C-terminal peptide (CTP) of the β subunit of human chorionic gonadotropin, polyethylene glycol (PEG), long unstructured hydrophilic sequences of amino acids (XTEN), hydroxyethyl starch (HES), an albumin-binding small molecule, or a combination thereof.

In some aspects, the half-life extending moiety is an Fc. In certain aspects, the Fc is a hybrid Fc, comprising a hinge region, a CH2 domain, and a CH3 domain, wherein the hinge region comprises a human IgD hinge region, wherein the CH2 domain comprises a part of human IgD CH2 domain and a part of human IgG4 CH2 domain, and wherein the CH3 domain comprises a part of human IgG4 CH3 domain.

In some aspects, the IL-7 protein comprises an amino acid sequence having a sequence identity of at least about 70%, at least about 75%, at least about 80%, at least about 85%, at least about 90%, at least about 95%, at least about 96%, at least about 97%, at least about 98%, at least about 99%, or about 100% to SEQ ID NOs: 1-6 and 15-25.

In some aspects, the CAR-bearing immune cells are autologous. In some aspects, the CAR-bearing immune cells are allogenic.

In some aspects, the CAR comprises an antigen-binding domain, which binds to a solid tumor antigen. In some aspects, the solid tumor antigen comprises mesothelin, MR1, guanylate cyclase C (GC-C), epidermal growth factor receptor (EGFR or erbB-1), human epidermal growth factor receptor 2 (HER2 or erbB2), erbB-3, erbB-4, MUC-1, melanoma-associated chondroitin sulfate proteoglycan (MCSP), folate receptor 1 (FOLR1), CD4, CD19, CD20, CD22, CD30, CD33, CD38, CD44, CD44v6, CD44v7/8, CD70, CD123, CD138, CD171, CEA, CSPG4, CXCR5, c-Met, FIERY-envelope protein, eriostin, Bigh3, SPARC, BCR, CD79, CD37, EGFRvIII, EGP2, EGP40, IGFr, L1CAM, AXL, Tissue Factor (TF), CD74, EpCAM, EphA2, MRP3cadherin 19 (CDH19), epidermal growth factor 2 (HER2), 5T4, 8H9, a436 integrin, BCMA, B7-H3, B7-H6, CAIX, CA9, FAP, FBP, fetal AchR, FRcc, GD2, GD3, Glypican-1 (GPC1), Glypican-2 (GPC2), Glypican-3 (GPC3), HLA-A1+MAGE1, HLA-A1+NY-ESO-1, IL-13Rcc2, Lewis-Y, KDR, MCSP, Muc1, Muc16, NCAM, NKG2D ligands, NY-ESO-1, PRAME, PSC1, PSCA, PSMA, ROR1, ROR2, SP17, surviving, TAG72, TEMs, carcinoembryonic antigen, HMW-MAA, VEGF, CLDN18.2, or combinations thereof.

In some aspects, the CAR comprises an antigen binding domain, which binds to one or more antigens selected from CD2, CD3ε, CD4, CD5, CD7, CD19, TRAC, BCMA, TCRβ, or combinations thereof.

In some aspects, the CAR-bearing immune cells comprise CAR-T cells, CAR-iNKT cells, or both. In certain aspects, the CAR-bearing immune cells are CAR-T cells. In some aspects, the CAR-T cells are genome edited CAR-T cells. In some aspects, the genome-edited CAR-T cells comprise a deletion or modification in one or more antigens selected from CD2, CDR3ε, CD4, CD5, CD7, TRAC, TCRβ, or combinations thereof. In some aspects, the CAR-T cells are dual CAR-T cells or tandem CAR-T cells. In some aspects, the CAR-bearing immune cells are CAR-iNKT cells. In some aspects, the CAR-iNKT cells are genome edited CAR-iNKT cells. In some aspects, the genome-edited CAR-iNKT cells comprise a deletion or modification in one or more antigens selected from CD2, CD3ε, CD4, CD5, CD7, TRAC, TCRβ, or combinations thereof. In some aspects, the CAR-iNKT cells are dual CAR-iNKT cells or tandem CAR-iNKT cells.

Provided herein is a method of treating a solid tumor in a subject in need thereof, comprising administering to the subject a population of immune cells comprising a transgenic T-cell receptor (TCR) (“transgenic TCR-bearing immune cells”) in combination with an IL-7 protein. Also provided is a method of enhancing an anti-tumor activity of immune cells comprising a transgenic T-cell receptor (TCR) (“transgenic TCR-bearing immune cells”) in a subject having a solid tumor, comprising administering to the subject a population of the transgenic TCR-bearing immune cells in combination with an IL-7 protein. Present disclosure provides a method of increasing an expansion of immune cells comprising a transgenic T-cell receptor (TCR) (“transgenic TCR-bearing immune cells”) in a subject having a solid tumor, comprising administering to the subject a population of the transgenic TCR-bearing immune cells in combination with an IL-7 protein. Present disclosure further provides method of increasing a survival and/or persistence of immune cells comprising a transgenic T-cell receptor (TCR) (“transgenic TCR-bearing immune cells”) in a subject having a solid tumor, comprising administering to the subject a population of the transgenic TCR-bearing immune cells in combination with an IL-7 protein.

In some aspects, the solid tumor is derived from a mesothelioma, cervical cancer, pancreatic cancer, ovarian cancer, squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulvar cancer, thyroid cancer, hepatic carcinoma, anal carcinoma, penile carcinoma head and neck cancer, or any combination thereof.

In some aspects, methods disclosed herein further comprise administering at least one additional therapeutic agent to the subject. In certain aspects, the additional therapeutic agent comprises an immune checkpoint inhibitor. In some aspects, the immune checkpoint inhibitor comprises an inhibitor of PD-1, PD-L1, LAG-3, Tim-3, CTLA-4, or any combination thereof.

### BRIEF DESCRIPTION OF THE FIGURES

FIGS. 1A, 1B, and 1C show the anti-tumor effects of mesothelin-specific CAR-T cells, alone or in combination with IL-7 protein, in a mouse model of pancreatic cancer. The animals were injected with GFP-labeled AsPC-1 tumor cells and then subsequently treated with one of the following: (i) buffer+vehicle control (“tumor only”; triangle); (ii) IL-7 protein+vehicle control (“tumor+IL-7”; inverted triangle); (iii) buffer+CAR-T cells (“tumor+mesoCAR”; closed circle); and (iv) IL-7+CAR-T cells (“tumor+mesoCAR+IL-7”; open circle). FIGS. 1A and 1B provide a comparison of tumor burden as measured by bioluminescence assay and caliper, respectively. FIG. 1C provide a comparison of the survival data.

FIGS. 2A, 2B, and 2C show the anti-tumor effects of mesothelin-specific CAR-T cells, alone or in combination with IL-7 protein, in a mouse model of pancreatic cancer. The animals were injected with GFP-labeled AsPC-1 tumor cells and then subsequently treated with one of the following: (i) buffer+vehicle control (“tumor only”; triangle); (ii) IL-7 protein+vehicle control (“tumor+IL-7”; inverted triangle); (iii) buffer+CAR-T cells (“tumor+mesoCAR”; closed circle); and (iv) IL-7+CAR-T cells (“tumor+mesoCAR+IL-7”; open circle). FIGS. 2A and 2B provide a comparison of tumor burden as measured by bioluminescence assay and caliper, respectively. FIG. 2C provide a comparison of the survival data.

### DETAILED DESCRIPTION OF THE DISCLOSURE

### I. Definitions

In order that the present disclosure can be more readily understood, certain terms are first defined. As used in this application, except as otherwise expressly provided herein, each of the following terms shall have the meaning set forth below. Additional definitions are set forth throughout the application.

Throughout this disclosure, the term “a” or “an” entity refers to one or more of that entity; for example, “an antibody,” is understood to represent one or more antibodies. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

As used herein, “administering” refers to the physical introduction of a therapeutic agent or a composition comprising a therapeutic agent to a subject, using any of the various methods and delivery systems known to those skilled in the art. The different routes of administration for a therapeutic agent described herein include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, intratracheal, pulmonary, subcutaneous, subcuticular, intraarticular, subcapsular, subarachnoid, intraventricle, intravitreal, epidural, and intrasternal injection and infusion, as well as in vivo electroporation. Alternatively, a therapeutic agent described herein can be administered via a non-parenteral route, such as a topical, epidermal, or mucosal route of administration, for example, intranasally, orally, vaginally, rectally, sublingually, or topically. Administering can also be performed, for example, once, a plurality of times, and/or over one or more extended periods.

As used herein, the term “antigen” refers to any natural or synthetic immunogenic substance, such as a protein, peptide, or hapten. In certain aspects, the antigen comprises a tumor antigen. The term “tumor antigen” refers to an antigen that is uniquely or differentially expressed on a tumor cell compared to normal healthy cells. In some aspects, the tumor antigen is expressed on a solid tumor (i.e., “solid tumor antigen”).

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

A “polypeptide” refers to a chain comprising at least two consecutively linked amino acid residues, with no upper limit on the length of the chain. One or more amino acid residues in the protein can contain a modification such as, but not limited to, glycosylation, phosphorylation or disulfide bond formation. A “protein” can comprise one or more polypeptides. Unless otherwise specified, the terms “protein” and “polypeptide” can be used interchangeably.

The term “nucleic acid molecule,” as used herein, is intended to include DNA molecules and RNA molecules. A nucleic acid molecule can be single-stranded or double-stranded, and can be cDNA.

The nucleic acids can be present in whole cells, in a cell lysate, or in a partially purified or substantially pure form. A nucleic acid is “isolated” or “rendered substantially pure” when purified away from other cellular components or other contaminants, e.g., other cellular nucleic acids (e.g., the other parts of the chromosome) or proteins, by standard techniques, including alkaline/SDS treatment, CsCl banding, column chromatography, agarose gel electrophoresis and others well known in the art. See, F. Ausubel, et al., ed. Current Protocols in Molecular Biology, Greene Publishing and Wiley Interscience, New York (1987).

Nucleic acids, e.g., cDNA, can be mutated, in accordance with standard techniques to provide gene sequences. For coding sequences, these mutations, can affect amino acid sequence as desired. In particular, DNA sequences substantially homologous to or derived from native V, D, J, constant, switches and other such sequences described herein are contemplated (where “derived” indicates that a sequence is identical or modified from another sequence).

“Conservative amino acid substitutions” refer to substitutions of an amino acid residue with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). In certain aspects, a predicted nonessential amino acid residue in an antibody is replaced with another amino acid residue from the same side chain family. Methods of identifying nucleotide and amino acid conservative substitutions which do not eliminate antigen binding are well-known in the art (see, e.g., Brummell et al., Biochem. 32: 1180-1187 (1993); Kobayashi et al. Protein Eng. 12(10):879-884 (1999); and Burks et al. Proc. Natl. Acad. Sci. USA 94:412-417 (1997)).

For nucleic acids, the term “substantial homology” indicates that two nucleic acids, or designated sequences thereof, when optimally aligned and compared, are identical, with appropriate nucleotide insertions or deletions, in at least about 80% of the nucleotides, at least about 90% to 95%, or at least about 98% to 99.5% of the nucleotides. Alternatively, substantial homology exists when the segments will hybridize under selective hybridization conditions, to the complement of the strand.

For polypeptides, the term “substantial homology” indicates that two polypeptides, or designated sequences thereof, when optimally aligned and compared, are identical, with appropriate amino acid insertions or deletions, in at least about 80% of the amino acids, at least about 90% to 95%, or at least about 98% to 99.5% of the amino acids.

The percent identity between two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, e.g., as described in the non-limiting examples below.

The percent identity between two nucleotide sequences can be determined using the GAP program in the GCG software package (available at worldwideweb.gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4: 11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48):444-453 (1970)) algorithm which has been incorporated into the GAP program in the GCG software package (available at worldwideweb.gcg.com), using either a Blossum 62 matrix or a PAM250 matrix, and a gap weight of 16, 14, 12, 10, 8, 6, or 4 and a length weight of 1, 2, 3, 4, 5, or 6.

The nucleic acid and protein sequences described herein can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to the nucleic acid molecules described herein. BLAST protein searches can be performed with the XBLAST program, score=50, wordlength=3 to obtain amino acid sequences homologous to the protein molecules described herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See worldwideweb.ncbi.nlm.nih.gov.

As used herein, the term “effector function” refers to a specialized function of a differentiated immune cell. An effector function of a T cell, for example, can be cytolytic activity or helper activity including the secretion of cytokines. An effector function in a naive, memory, or memory-type T cell can also include antigen-dependent proliferation.

The term “fratricide” as used herein means a process which occurs when a CAR-T cell or iNKT-CAR cell becomes the target of, and is killed by, another CAR-T cell or iNKT-CAR cell comprising the same chimeric antigen receptor as the target of CAR-T or iNKT-CAR cell, because the targeted cell expresses the antigen specifically recognized by the chimeric antigen receptor on both cells. CAR-T cells or iNKT-CARs comprising a chimeric antigen receptor which are deficient in an antigen to which the chimeric antigen receptor specifically binds will be “fratricide-resistant.”

The term “immune cell,” as used herein, refers to cells that play a role in the immune response. Accordingly, in some aspects, immune cells useful for the present disclosure are those cells that can play a role in the treatment and/or eradication of a solid tumor (e.g., possess anti-tumor activity). In some aspects, the immune cells comprise lymphocytes, neutrophils, monocytes, macrophages, dendritic cells, or any combination thereof. In certain aspects, the lymphocytes comprise T cells, tumor-infiltrating lymphocytes (TIL), lymphokine-activated killer cells, natural killer T (NKT) cells, or any combination thereof. In some aspects, the lymphocytes are T cells. In some aspects, the lymphocytes are NKT cells (e.g., invariant NKT cells).

As described herein, immune cells useful for the present disclosure have been modified (i.e., modified immune cells). As used herein, the term “modified immune cell” refers to an immune cell that has been altered (e.g., genetically), such that the cell exhibits one or more properties that are not present in a corresponding non-modified immune cell. For example, in some aspects, immune cells have been modified to express a chimeric antigen receptor disclosed herein (e.g., CAR-T cells or iNKT-CAR cells). In some aspects, immune cells have been modified to express a transgenic T-cell receptor (e.g., engineered T cells). As used herein, the term “transgenic T-cell receptor” refers to a T-cell receptor (TCR) that has been modified, such that the TCR differs in one or more properties from the corresponding non-modified TCR. For instance, in some aspects, the transgenic T-cell receptor is expressed on an immune cell that does not naturally express the TCR (e.g., a transgenic TCR specific for antigen A is expressed in T cells specific for antigen B). In some aspects, the transgenic TCR has engineered to specifically bind with a desired affinity to a target antigen (e.g., solid tumor antigen). In some aspects, transgenic TCRs can refer to TCRs with desired affinity to a target antigen and expressed on immune cells that do not naturally express the TCRs. Further modifications that can be made to immune cells are provided elsewhere in the present disclosure (e.g., fratricide-resistant).

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”) In general, expression vectors of utility in recombinant DNA techniques are often in the form of plasmids. In the present specification, “plasmid” and “vector” can be used interchangeably as the plasmid is the most commonly used form of vector. However, also included are other forms of expression vectors, such as viral vectors (e.g., replication defective retroviruses, adenoviruses and adeno-associated viruses), which serve equivalent functions.

The term “recombinant host cell” (or simply “host cell”), as used herein, is intended to refer to a cell that comprises a nucleic acid that is not naturally present in the cell, and can be a cell into which a recombinant expression vector has been introduced. It should be understood that such terms are intended to refer not only to the particular subject cell but to the progeny of such a cell. Because certain modifications can occur in succeeding generations due to either mutation or environmental influences, such progeny cannot, in fact, be identical to the parent cell, but are still included within the scope of the term “host cell” as used herein.

As used herein, the term “linked” refers to the association of two or more molecules. The linkage can be covalent or non-covalent. The linkage also can be genetic (i.e., recombinantly fused). Such linkages can be achieved using a wide variety of art recognized techniques, such as chemical conjugation and recombinant protein production.

A “cancer” refers a broad group of various diseases characterized by the uncontrolled growth of abnormal cells in the body. Unregulated cell division and growth results in the formation of malignant tumors that invade neighboring tissues and can also metastasize to distant parts of the body through the lymphatic system or bloodstream. Cancers that can be treated with the present disclosure include those associated with a solid tumor.

As used herein, the term “solid tumor” refers to an abnormal mass of tissue that usually does not contain cysts or liquid areas. Different types of solid tumors are named for the particular cells that form them, for example, sarcomas are formed from connective tissue cells (e.g., bone cartilage or fat), carcinomas are formed from epithelial tissue cells (e.g., breast, colon, or pancreas), and lymphomas are formed from lymphatic tissue cells (e.g., lymph nodes, spleen, or thymus). As described herein, the present disclosure can be used to treat all types of solid tumors. In some aspects, cancers comprise primary, metastatic, and/or recurrent cancers.

The term “fusion protein” refers to proteins created through the joining of two or more genes that originally coded for separate proteins. Translation of this fusion gene results in a single polypeptide or multiple polypeptides with functional properties derived from each of the original proteins. In some aspects, the two or more genes can comprise a substitution, a deletion, and/or an addition in its nucleotide sequence.

An “Fc receptor” or “FcR” is a receptor that binds to the Fc region of an immunoglobulin. FcRs that bind to an IgG antibody comprise receptors of the FcγR family, including allelic variants and alternatively spliced forms of these receptors. The FcγR family consists of three activating (FcγRI, FcγRIII, and FcγRIV in mice; FcγRIA, FcγRIIA, and FcγRIIIA in humans) and one inhibitory (FcγRIIB) receptor. Various properties of human FcγRs are known in the art. The majority of innate effector cell types coexpress one or more activating FcγR and the inhibitory FcγRIIB, whereas natural killer (NK) cells selectively express one activating Fc receptor (FcγRIII in mice and FcγRIIIA in humans) but not the inhibitory FcγRIIB in mice and humans. Human IgG1 binds to most human Fc receptors and is considered equivalent to murine IgG2a with respect to the types of activating Fc receptors that it binds to.

An “Fc region” (fragment crystallizable region) or “Fc domain” or “Fc” refers to the C-terminal region of the heavy chain of an antibody that mediates the binding of the immunoglobulin to host tissues or factors, including binding to Fc receptors located on various cells of the immune system (e.g., effector cells) or to the first component (C1q) of the classical complement system. Thus, an Fc region comprises the constant region of an antibody excluding the first constant region immunoglobulin domain (e.g., CH1 or CL). In IgG, IgA and IgD antibody isotypes, the Fc region comprises two identical protein fragments, derived from the second (CH2) and third (CH3) constant domains of the antibody's two heavy chains; IgM and IgE Fc regions comprise three heavy chain constant domains (CH domains 2-4) in each polypeptide chain. For IgG, the Fc region comprises immunoglobulin domains CH2 and CH3 and the hinge between CH1 and CH2 domains. Although the definition of the boundaries of the Fc region of an immunoglobulin heavy chain might vary, as defined herein, the human IgG heavy chain Fc region is defined to stretch from an amino acid residue D221 for IgG1, V222 for IgG2, L221 for IgG3 and P224 for IgG4 to the carboxy-terminus of the heavy chain, wherein the numbering is according to the EU index as in Kabat. The CH2 domain of a human IgG Fc region extends from amino acid 237 to amino acid 340, and the CH3 domain is positioned on C-terminal side of a CH2 domain in an Fc region, i.e., it extends from amino acid 341 to amino acid 447 or 446 (if the C-terminal lysine residue is absent) or 445 (if the C-terminal glycine and lysine residues are absent) of an IgG. As used herein, the Fc region can be a native sequence Fc, including any allotypic variant, or a variant Fc (e.g., a non-naturally occurring Fc). Fc can also refer to this region in isolation or in the context of an Fc-comprising protein polypeptide such as a “binding protein comprising an Fc region,” also referred to as an “Fc fusion protein” (e.g., an antibody or immunoadhesion).

A “native sequence Fc region” or “native sequence Fc” comprises an amino acid sequence that is identical to the amino acid sequence of an Fc region found in nature. Native sequence human Fc regions include a native sequence human IgG1 Fc region; native sequence human IgG2 Fc region; native sequence human IgG3 Fc region; and native sequence human IgG4 Fc region as well as naturally occurring variants thereof. Native sequence Fc include the various allotypes of Fcs (see, e.g., Jefferis et al. (2009) mAbs 1: 1).

Additionally, an Fc (native or variant) of the present disclosure can be in the form of having native sugar chains, increased sugar chains, or decreased sugar chains compared to the native form, or may be in a deglycosylated form. The immunoglobulin Fc sugar chains can be modified by conventional methods such as a chemical method, an enzymatic method, and a genetic engineering method using a microorganism. The removal of sugar chains from an Fc fragment results in a sharp decrease in binding affinity to the Clq part of the first complement component C1, and a decrease or loss of ADCC or CDC, thereby not inducing any unnecessary immune responses in vivo. In this regard, an immunoglobulin Fc region in a deglycosylated or aglycosylated form may be more suitable to the object of the present disclosure as a drug carrier. As used herein, the term “deglycosylation” refers to an Fc region in which sugars are removed enzymatically from an Fc fragment. Additionally, the term “aglycosylation” means that an Fc fragment is produced in an unglycosylated form by a prokaryote, and preferably in E. coli.

As used herein, the term “immune response” refers to a biological response within a vertebrate against foreign agents, which response protects the organism against these agents and diseases caused by them. An immune response is mediated by the action of a cell of the immune system (e.g., a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell or a Th cell, such as a CD4⁺ or CD8⁺ T cell, or the inhibition of a Treg cell.

An “immunomodulator” or “immunoregulator” refers to an agent, e.g., a component of a signaling pathway, that can be involved in modulating, regulating, or modifying an immune response. “Modulating,” “regulating,” or “modifying” an immune response refers to any alteration in a cell of the immune system or in the activity of such cell (e.g., an effector T cell). Such modulation includes stimulation or suppression of the immune system which can be manifested by an increase or decrease in the number of various cell types, an increase or decrease in the activity of these cells, or any other changes which can occur within the immune system. Both inhibitory and stimulatory immunomodulators have been identified, some of which can have enhanced function in a tumor microenvironment. In preferred aspects, the immunomodulator is located on the surface of a T cell. An “immunomodulatory target” or “immunoregulatory target” is an immunomodulator that is targeted for binding by, and whose activity is altered by the binding of, a substance, agent, moiety, compound or molecule. Immunomodulatory targets include, for example, receptors on the surface of a cell (“immunomodulatory receptors”) and receptor ligands (“immunomodulatory ligands”).

The term “immunotherapy” refers to the treatment of a subject afflicted with, or at risk of contracting or suffering a recurrence of, a disease by a method comprising inducing, enhancing, suppressing or otherwise modifying an immune response. “Treatment” or “therapy” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease.

“Immunostimulating therapy” or “immunostimulatory therapy” refers to a therapy that results in increasing (inducing or enhancing) an immune response in a subject for, e.g., treating cancer.

The term “effector T cells” (Teff) refers to T cells (e.g., CD4⁺ and CD8⁺ T cells) with cytolytic activities as well as T helper (Th) cells, which secrete cytokines and activate and direct other immune cells, but does not include regulatory T cells (Treg cells). In some aspects, combination of an IL-7 protein and a population of CAR-bearing immune cells can activate and/or increase the frequency of Teff cells, e.g., CD4⁺ and CD8⁺ T cells, in a solid tumor of a subject. In some aspects, combination of an IL-7 protein and a population of transgenic TCR-bearing immune cells can activate and/or increase the frequency of Teff cells, e.g., CD4⁺ and CD8⁺ T cells, in a solid tumor of a subject.

As used herein, the term “regulatory T cells” (Tregs) refer to a population of T cells with the ability to reduce or suppress the induction and proliferation of effector T cells, and thereby, modulate an immune response. In some aspects, Tregs can suppress an immune response by secreting anti-inflammatory cytokines, such as IL-10, TGF-β, and IL-35, which can interfere with the activation and differentiation of naïve T cells into effector T cells. In some aspects, Tregs can also produce cytolytic molecules, such as Granzyme B, which can induce the apoptosis of effector T cells. In some aspects, the regulatory T cells are natural regulatory T cells (nTregs) (i.e., developed within the thymus). In some aspects, the regulatory T cells are induced regulatory T cells (iTregs) (i.e., naïve T cells that differentiate into Tregs in the peripheral tissue upon exposure to certain stimuli). Methods for identifying Tregs are known in the art. For example, Tregs express certain phenotypic markers (e.g., CD25, Foxp3, or CD39) that can be measured using flow cytometry. See, e.g., International Publication No. WO 2017/062035 A1; Gu J., et al., Cell Mol Immunol 14(6): 521-528 (2017). In some aspects, the Tregs are CD45RA⁻ CD39⁺ T cells.

As used herein, the term “tumor infiltrating lymphocytes” or “TILs” refers to lymphocytes (e.g., effector T cells) that have migrated from the periphery (e.g., from the blood) into a tumor. In some aspects, the tumor infiltrating lymphocytes are CD4+ TILs. In some aspects, the tumor infiltrating lymphocytes are CD8+ TILs.

An increased ability to stimulate an immune response or the immune system, can result from an enhanced agonist activity of T cell costimulatory receptors and/or an enhanced antagonist activity of inhibitory receptors. An increased ability to stimulate an immune response or the immune system can be reflected by a fold increase of the EC50 or maximal level of activity in an assay that measures an immune response, e.g., an assay that measures changes in cytokine or chemokine release, cytolytic activity (determined directly on target cells or indirectly via detecting CD107a or granzymes) and proliferation. The ability to stimulate an immune response or the immune system activity can be enhanced by at least 10%, 30%, 50%, 75%, 2 fold, 3 fold, 5 fold or more.

As used herein, the term “interleukin-7” or “IL-7” refers to IL-7 polypeptides and derivatives and analogs thereof having substantial amino acid sequence identity to wild-type mature mammalian IL-7 proteins and substantially equivalent biological activity, e.g., in standard bioassays or assays of IL-7 receptor binding affinity. Additional disclosure relating to IL-7 proteins that can be used with the present disclosure are provided elsewhere herein.

A “variant” of an IL-7 protein is defined as an amino acid sequence that is altered by one or more amino acids. The variant can have “conservative” changes, wherein a substituted amino acid has similar structural or chemical properties, e.g., replacement of leucine with isoleucine. More rarely, a variant can have “nonconservative” changes, e.g., replacement of a glycine with a tryptophan. Similar minor variations can also include amino acid deletions or insertions, or both. Guidance in determining which and how many amino acid residues may be substituted, inserted or deleted without abolishing biological activity can be found using computer programs well known in the art, for example software for molecular modeling or for producing alignments. The variant IL-7 proteins included within the present disclosure include IL-7 proteins that retain IL-7 activity. IL-7 polypeptides which also include additions, substitutions or deletions are also included within the present disclosure as long as the proteins retain substantially equivalent biological IL-7 activity. For example, truncations of IL-7 which retain comparable biological activity as the full length form of the IL-7 protein are included within the present disclosure. In some aspects, variant IL-7 proteins also include polypeptides that have at least about 70%, at least about 75%, at least about 80%, at least about 85%, at least about 90%, at least about 91%, at least about 92%, at least about 93%, at least about 93%, at least about 95%, at least about 96%, at least about 97%, at least about 98%, at least about 99%, or more sequence identity with wild-type IL-7.

As used herein, the term “signal sequence,” or equivalently, “signal peptide,” refers to a fragment directing the secretion of a biologically active molecule drug and a fusion protein, and it is cut off after being translated in a host cell. The signal sequence as used herein is a polynucleotide encoding an amino acid sequence initiating the movement of the protein penetrating the endoplasmic reticulum (ER) membrane. Useful signal sequences include an antibody light chain signal sequence, e.g., antibody 14.18 (Gillies et al., J. Immunol. Meth 1989. 125:191-202), an antibody heavy chain signal sequence, e.g., MOPC141 an antibody heavy chain signal sequence (Sakano et al., Nature, 1980.286: 676-683), and other signal sequences know in the art (e.g., see Watson et al., Nucleic Acid Research, 1984.12:5145-5164). The characteristics of signal peptides are well known in the art, and the signal peptides conventionally having 16 to 30 amino acids, but they can include more or less number of amino acid residues. Conventional signal peptides consist of three regions of the basic N-terminal region, a central hydrophobic region, and a more polar C-terminal region.

A “subject” includes any human or nonhuman animal. The term “nonhuman animal” includes, but is not limited to, vertebrates such as nonhuman primates, sheep, dogs, and rodents such as mice, rats and guinea pigs. In some aspects, the subject is a human. The terms “subject” and “patient” are used interchangeably herein.

The term “therapeutically effective amount” or “therapeutically effective dosage” refers to an amount of an agent that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In reference to solid tumors, an effective amount comprises an amount sufficient to cause a tumor to shrink and/or to decrease the growth rate of the tumor (such as to suppress tumor growth) or to prevent or delay other unwanted cell proliferation. In some aspects, an effective amount is an amount sufficient to delay tumor development. In some aspects, an effective amount is an amount sufficient to prevent or delay tumor recurrence. An effective amount can be administered in one or more administrations. The effective amount of the drug or composition can: (i) reduce the number of cancer cells; (ii) reduce tumor size; (iii) inhibit, retard, slow to some extent and can stop cancer cell infiltration into peripheral organs; (iv) inhibit (i.e., slow to some extent and can stop tumor metastasis; (v) inhibit tumor growth; (vi) prevent or delay occurrence and/or recurrence of tumor; and/or (vii) relieve to some extent one or more of the symptoms associated with the cancer. In some aspects, a “therapeutically effective amount” is the amount of IL-7 protein and the amount of a population of modified immune cells disclosed herein (e.g., CAR-bearing immune cells and/or transgenic TCR-bearing immune cells), in combination, clinically proven to affect a significant decrease in cancer or slowing of progression (regression) of cancer, such as an advanced solid tumor. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

The term “dosing frequency” refers to the number of times a therapeutic agent (e.g., an IL-7 protein, a population of CAR-bearing immune cells, or a population of transgenic TCR-bearing immune cells) is administered to a subject within a specific time period. Dosing frequency can be indicated as the number of doses per a given time, for example, once per day, once a week, or once in two weeks. As used herein, “dosing frequency” is applicable where a subject receives multiple (or repeated) administrations of a therapeutic agent.

As used herein, the term “standard of care” refers to a treatment that is accepted by medical experts as a proper treatment for a certain type of disease and that is widely used by healthcare professionals. The term can be used interchangeable with any of the following terms: “best practice,” “standard medical care,” and “standard therapy.”

By way of example, an “anti-cancer agent” promotes cancer regression in a subject or prevents further tumor growth. In certain aspects, a therapeutically effective amount of the drug promotes cancer regression to the point of eliminating the cancer. “Promoting cancer regression” means that administering an effective amount of the drug, alone or in combination with an anti-neoplastic agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug.

By way of example, for the treatment of solid tumors, a therapeutically effective amount of an anti-cancer agent can inhibit cell growth or tumor growth by at least about 10%, at least about 20%, by at least about 40%, by at least about 60%, or by at least about 80% relative to untreated subjects or, in certain aspects, relative to patients treated with a standard-of-care therapy. In some aspects, solid tumor regression can be observed and continue for a period of at least about 20 days, at least about 40 days, or at least about 60 days. Notwithstanding these ultimate measurements of therapeutic effectiveness, evaluation of immunotherapeutic drugs must also make allowance for “immune-related” response patterns.

As used herein, the term “immune checkpoint inhibitor” refers to molecules that totally or partially reduce, inhibit, interfere with or modulate one or more immune checkpoint proteins. Immune checkpoint proteins regulate T-cell activation or function. Numerous checkpoint proteins are known, such as CTLA-4 and its ligands CD80 and CD86; and PD-1 with its ligands PD-L1 and PD-L2. Pardoll, D. M., Nat Rev Cancer 12(4):252-64 (2012). These proteins are responsible for co-stimulatory or inhibitory interactions of T-cell responses. Immune checkpoint proteins regulate and maintain self-tolerance and the duration and amplitude of physiological immune responses. Immune checkpoint inhibitors include antibodies or are derived from antibodies.

The term “reference,” as used herein, refers to a corresponding subject (e.g., a subject having a solid tumor) who did not receive a combination of an IL-7 protein and a population of modified immune cells disclosed herein (e.g., CAR-bearing immune cells and/or transgenic TCR-bearing immune cells), e.g., a subject who received an IL-7 protein alone or a population of CAR-bearing or transgenic TCR-bearing immune cells alone. In some aspects, the reference subject received neither an IL-7 protein nor a population of modified immune cells (e.g., CAR-bearing immune cells and/or transgenic TCR-bearing immune cells). The term “reference” can also refer to a same cancer subject but prior to the administration of a combination therapy disclosed herein (i.e., IL-7 protein in combination with a population of CAR-bearing or transgenic TCR-bearing immune cells). In certain aspects, the term “reference” refers to an average of a population of subjects (e.g., subjects having a solid tumor).

As used herein, the terms “ug” and “uM” are used interchangeably with “μg” and “μM,” respectively.

Various aspects described herein are described in further detail in the following subsections.

### II. Methods of the Disclosure

The present disclosure is directed to a method for treating a solid tumor (or a cancer associated with a solid tumor) in a subject in need thereof, comprising administering to the subject an effective amount of an interleukin-7 (IL-7) protein in combination with an effective amount of a population of modified immune cells disclosed herein (e.g., chimeric antigen receptor (CAR)-bearing immune cells or transgenic T-cell receptor (TCR)-bearing immune cells). As described herein, CAR-bearing immune cells and/or transgenic TCR-bearing immune cells that are useful for the present disclosure can bind to an antigen expressed on a solid tumor (i.e., solid tumor antigen). Additional disclosure relating to exemplary IL-7 protein and modified immune cells (e.g., CAR-bearing immune cells and transgenic TCR-bearing immune cells) that can be used are provided elsewhere in the present disclosure.

In some aspects, treating a solid tumor comprises reducing the size of the solid tumor (i.e., tumor volume). In certain aspects, administering an IL-7 protein in combination with a population of CAR-bearing immune cells to a subject having a solid tumor can decrease the tumor volume by at least about 5%, at least about 10%, at least about 20%, at least about 30%, at least about 40%, at least about 50%, at least about 60%, at least about 70%, at least about 80%, at least about 90%, or about 100% compared to a reference tumor volume (e.g., tumor volume in a corresponding subject that received either IL-7 protein alone or population of CAR-bearing immune cells alone). In certain aspects, administering an IL-7 protein in combination with a population of transgenic TCR-bearing immune cells to a subject having a solid tumor can decrease the tumor volume by at least about 5%, at least about 10%, at least about 20%, at least about 30%, at least about 40%, at least about 50%, at least about 60%, at least about 70%, at least about 80%, at least about 90%, or about 100% compared to a reference tumor volume (e.g., tumor volume in a corresponding subject that received either IL-7 protein alone or population of transgenic TCR-bearing immune cells alone).

In some aspects, treating a solid tumor comprises prolonging (i.e., increasing) the survival of a subject having a solid tumor. In certain aspects, administering an IL-7 protein in combination with a population of CAR-bearing immune cells to a subject having a solid tumor can increase the duration of survival of the subject by at least about 5%, at least about 10%, at least about 20%, at least about 30%, at least about 40%, at least about 50%, at least about 60%, at least about 70%, at least about 80%, at least about 90%, at least about 100%, at least about 150%, or at least about 200% or more compared to a reference duration of survival (e.g., survival in a corresponding subject that received either IL-7 protein alone or CAR-bearing immune cells alone). In some aspects, the duration of survival of the subject (i.e., treated with an IL-7 protein in combination with a population of CAR-bearing immune cells) is increased by at least about 10 days, at least about 20 days, at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 1 year or more.

In some aspects, administering an IL-7 protein in combination with a population of transgenic TCR-bearing immune cells to a subject having a solid tumor can increase the duration of survival of the subject by at least about 5%, at least about 10%, at least about 20%, at least about 30%, at least about 40%, at least about 50%, at least about 60%, at least about 70%, at least about 80%, at least about 90%, at least about 100%, at least about 150%, or at least about 200% or more compared to a reference duration of survival (e.g., survival in a corresponding subject that received either IL-7 protein alone or population of transgenic TCR-bearing immune cells alone). In some aspects, the duration of survival of the subject (i.e., treated with an IL-7 protein in combination with a population of transgenic TCR-bearing immune cells) is increased by at least about 10 days, at least about 20 days, at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 1 year or more.

In some aspects, administering a combination therapy of the present disclosure (i.e., IL-7 protein in combination with a population of modified immune cells, e.g., CAR-bearing immune cells and/or transgenic TCR-bearing immune cells) increase the duration of progression-free survival of a subject having a solid tumor. As used herein, the term “progression-free survival” refers to the length of time during and after the treatment of a disease (e.g., with an administration of an IL-7 protein in combination with a population of modified immune cells disclosed herein (e.g., CAR-bearing immune cells and/or transgenic TCR-bearing immune cells) that a subject lives with the disease but it does not get worse (e.g., the tumor does not grow in size). In some aspects, after administering a combination therapy disclosed herein, the progression free survival of the subject is increased by at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 1 year when compared to a reference subject (e.g., corresponding subject that received either IL-7 protein alone or modified immune cells (e.g., CAR-bearing immune cells or transgenic TCR-bearing immune cells) alone).

In some aspects, administering a combination therapy disclosed herein (i.e., IL-7 protein in combination with a population of modified immune cells, e.g., CAR-bearing immune cells or transgenic TCR-bearing immune cells) methods disclosed herein effectively increases the response rate in a group of subjects. For example, the response rate in a group of subjects is increased by at least about 2%, at least about 3%, at least about 4%, at least about 5%, at least about 10%, at least about 15%, at least about 20%, at least about 25%, at least about 30%, at last about 35%, at least about 40%, at least about 45%, at least about 50%, at least about 55%, at least about 60%, at least about 70%, at least about 75%, at least about 80%, at least about 85%, at least about 90%, at least about 95%, at least about 99% or at least about 100% when compared to a reference subject (e.g., corresponding subject treated with IL-7 protein alone or with a population of modified immune cells (e.g., CAR-bearing immune cells or transgenic TCR-bearing immune cells) alone).

In some aspects, administering an IL-7 protein in combination with a population of CAR-bearing immune cells can enhance and/or improve an anti-tumor activity of CAR-bearing immune cells in a subject having a solid tumor. In certain aspects, the anti-tumor activity of CAR-bearing immune cells is increased by at least about 5%, at least about 10%, at least about 20%, at least about 30%, at least about 40%, at least about 50%, at least about 60%, at least about 70%, at least about 80%, at least about 90%, at least about 100% or more compared to a reference (e.g., anti-tumor activity in a corresponding subject that received either IL-7 protein alone or CAR-bearing immune cells alone).

In some aspects, administering an IL-7 protein in combination with a population of transgenic TCR-bearing immune cells can enhance and/or improve an anti-tumor activity of transgenic TCR-bearing immune cells in a subject having a solid tumor. In certain aspects, the anti-tumor activity of transgenic TCR-bearing immune cells is increased by at least about 5%, at least about 10%, at least about 20%, at least about 30%, at least about 40%, at least about 50%, at least about 60%, at least about 70%, at least about 80%, at least about 90%, at least about 100% or more compared to a reference (e.g., anti-tumor activity in a corresponding subject that received either IL-7 protein alone or transgenic TCR-bearing immune cells alone).

In some aspects, anti-tumor activity of CAR-bearing immune cells and/or anti-tumor activity of transgenic TCR-bearing immune cells comprises the ability to reduce the size of the solid tumor (i.e., tumor volume) in the subject. In some aspects, anti-tumor activity comprises the ability to prolong the survival of a subject having a solid tumor. In some aspects, anti-tumor activity comprises the ability of the CAR-bearing immune cells and/or transgenic TCR-bearing immune cells to exhibit effector function upon activation (e.g., via stimulation with cognate antigen). As used herein, the term “effector function” refers to one or more properties of activated cells (e.g., CAR-bearing immune cells and/or transgenic TCR-bearing immune cells) that can help eradicate and/or kill a solid tumor. Non-limiting examples of effector functions include proliferation, expression of cytolytic molecules (e.g., perforin or granzymes), production of cytokines (e.g., TNF-α, IFN-γ, and/or IL-2), trafficking to tumor tissues, attenuating and/or resisting immune checkpoint inhibitory signaling (e.g., PD-1, LAG-3, TIM-3, or CTLA-4), or combinations thereof.

As will be apparent to those skilled in the art, anti-tumor activity of modified immune cells (e.g., CAR-bearing immune cells and/or transgenic TCR-bearing immune cells) disclosed herein can be measured by any relevant methods known in the art. For instance, the ability of the modified immune cells (e.g., CAR-bearing immune cells and/or TCR-transgenic immune cells) to reduce tumor volume and/or prolong the survival of a subject can be measured as described in the Examples. The ability of modified immune cells (e.g., CAR-bearing immune cells and/or transgenic TCR-bearing immune cells) to exhibit effector function can be measured using assays such as flow cytometry, ELISA, and/or ELISPOT.

As described herein, administering an IL-7 protein (e.g., those described herein) in combination with a population of modified immune cells (e.g., CAR-bearing immune cells and/or transgenic TCR-bearing immune cells) can increase the proliferation (i.e., expansion) of the modified immune cells (e.g., CAR-bearing immune cells and/or transgenic TCR-bearing immune cells) in a subject.

Accordingly, in some aspects, the present disclosure provides a method of increasing the expansion of CAR-bearing immune cells in a subject having a solid tumor, comprising administering to the subject a population of CAR-bearing immune cells in combination with an IL-7 protein. Also provided herein is a method of increasing the expansion of transgenic TCR-bearing immune cells in a subject having a solid tumor, comprising administering to the subject a population of transgenic TCR-bearing immune cells in combination with an IL-7 protein. In certain aspects, the expansion of the CAR-bearing immune cells and/or transgenic TCR-bearing immune cells is increased by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200% or more after the administration compared to a reference expansion (e.g., expansion of corresponding modified immune cells (i.e., CAR-bearing immune cells or transgenic TCR-bearing immune cells) in a subject that only received an administration of the population of modified immune cells (i.e., CAR-bearing immune cells or transgenic TCR-bearing immune cells).

In some aspects, an IL-7 protein disclosed herein can help increase the survival and/or persistence of modified immune cells disclosed herein (e.g., CAR-bearing immune cells and/or transgenic TCR-bearing immune cells) in a subject having a solid tumor when administered in combination with a population of the modified immune cells (e.g., CAR-bearing immune cells and/or transgenic TCR-bearing immune cells). In certain aspects, the survival and/or persistence of the modified immune cells (e.g., CAR-bearing immune cells and/or transgenic TCR-bearing immune cells) is increased by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200% or more when administered in combination with an IL-7 protein compared to a reference (e.g., survival and/or persistence of the modified immune cells (e.g., CAR-bearing immune cells and/or transgenic TCR-bearing immune cells) in a corresponding subject that only received an administration of the population of the modified immune cells)

In some aspects, the increased expansion, survival, and/or persistence of the modified immune cells (e.g., CAR-bearing immune cells and/or transgenic TCR-bearing immune cells) described above can result in an increase in the total number of modified immune cells (e.g., CAR-bearing immune cells and/or transgenic TCR-bearing immune cells) present in a subject having a solid tumor. In certain aspects, the total number of the modified immune cells (e.g., CAR-bearing immune cells and/or transgenic TCR-bearing immune cells) present in the subject is increased by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200% or more compared to a reference (e.g., the total number of the modified immune cells present in a corresponding subject that only received an administration of the population of modified immune cells, e.g., CAR-bearing immune cells and/or transgenic TCR-bearing immune cells).

As will be apparent to those skilled in the art, many cancer patients are lymphopenic, as many of the available standard of care cancer treatments (e.g., chemotherapy and radiation therapy) are known to cause lymphopenia. Accordingly, methods disclosed herein can also be used to treat a solid tumor (or a cancer associated with a solid tumor) in a lymphopenic subject.

As used herein, the term “lymphopenic subject” refers to a subject with lymphopenia. As used herein, the terms “lymphopenia” and “lymphocytopenia” are used interchangeably and refer to a condition characterized by abnormally low number of circulating immune cells (e.g., lymphocytes). Peripheral circulation of all types of lymphocytes or subpopulations of lymphocytes (e.g., CD4⁺ T cells) can be depleted or abnormally low in a patient suffering from lymphopenia. See, e.g., Lymphopenia Description, The Merck Manual (18th Edition, 2006, Merck & Co.). In some aspects, compared to a normal subject (e.g., healthy individual), a lymphopenic subject has reduced number of T-lymphocytes (“T-lymphopenia”), B-lymphocytes (“B-lymphopenia”), and/or NK cells (“NK lymphopenia”).

Quantitatively, lymphopenia can be described by various cutoffs. In some aspects, a lymphopenic subject has a circulating blood total lymphocyte count that is less than by at least about 5%, at least about 10%, at least about 20%, at least about 30%, at least about 40%, at least about 50%, at least about 60%, at least about 70%, at least about 80%, at least about 90%, or at least about 100% compared to a circulating blood total lymphocyte count in a corresponding subject who does not exhibit a lymphopenia. In some aspects, a subject has lymphopenia if the subject has a circulating blood total lymphocyte count of less than about 1,500 lymphocytes/μL, less than about 1,000 lymphocytes/μL, less than about 800 lymphocytes/μL, less than about 500 lymphocytes/μL, or less than about 200 lymphocytes/μL.

Lymphocytopenia has a wide range of possible causes. In some aspects, a lymphopenia is caused by or associated with a tumor. In some aspects, a lymphopenia is caused by or associated with a previous therapy for a tumor (e.g., chemotherapy or radiation therapy). In some aspects, a lymphopenia is caused by or associated with an infection, including viral (e.g., HIV or hepatitis infection), bacterial (e.g., active tuberculosis infection), and fungal infections; chronic failure of the right ventricle of the heart, Hodgkin's disease and cancers of the lymphatic system, leukemia, a leak or rupture in the thoracic duct, side effects of prescription medications including anticancer agents, antiviral agents, and glucocorticoids, malnutrition resulting from diets that are low in protein, radiation therapy, uremia, autoimmune disorders, immune deficiency syndromes, high stress levels, and trauma.

In some aspects, a lymphopenia is idiopathic (i.e., has unknown etiology). Non-limiting examples of idiopathic lymphopenia include idiopathic CD4 positive T-lymphocytopenia (ICL), acute radiation syndrome (ARS), or a combination thereof.

As described herein, the methods disclosed herein can be used to treat a solid tumor (or a cancer associated with a solid tumor). In some aspects, the solid tumor is derived from a mesothelioma, cervical cancer, pancreatic cancer, ovarian cancer, squamous cell cancer (e.g. epithelial squamous cell cancer), lung cancer (e.g., small-cell lung cancer (SCLC), non-small cell lung cancer, adenocarcinoma of the lung and squamous carcinoma of the lung), skin cancer (e.g., basal cell carcinoma (BCC), cutaneous squamous cell carcinoma (cSCC), melanoma, Merkel cell carcinoma (MCC)), cancer of the peritoneum, hepatocellular cancer, gastric or stomach cancer (e.g., gastrointestinal cancer), esophageal cancer (e.g., gastroesophageal junction cancer), pancreatic cancer, brain cancer (e.g., glioblastoma), liver cancer (e.g., hepatocellular carcinoma), bladder cancer, hepatoma, breast cancer (e.g., triple negative breast cancer (TNBC)), colon cancer, rectal cancer, colorectal cancer, endometrial cancer or uterine carcinoma, salivary gland carcinoma, kidney or renal cancer (e.g., renal cell carcinoma), prostate cancer, vulvar cancer, thyroid cancer, hepatic carcinoma, anal carcinoma, penile carcinoma, head and neck cancer (e.g., head and neck squamous cell carcinoma), or any combination thereof.

In some aspects, the methods described herein can also be used for treatment of metastatic cancers, unresectable, refractory cancers (e.g., cancers refractory to previous cancer therapy, e.g., immunotherapy, e.g., with a blocking anti-PD-1 antibody), and/or recurrent cancers. In certain aspects, the previous cancer therapy comprises a chemotherapy. In some aspects, the chemotherapy comprises a platinum-based therapy. In some aspects, the platinum-based therapy comprises a platinum-based antineoplastic selected from the group consisting of cisplatin, carboplatin, oxaliplatin, nedaplatin, triplatin tetranitrate, phenanthriplatin, picoplatin, satraplatin, and any combination thereof. In certain aspects, the platinum-based therapy comprises cisplatin. In certain aspects, the platinum-based therapy comprises carboplatin.

In some aspects, a subject to be treated with the methods disclosed herein has received one, two, three, four, five or more prior cancer treatments. In some aspects, the subject is treatment-naïve (i.e., has never received a prior cancer treatment). In some aspects, the subject has progressed on other cancer treatments. In certain aspects, the prior cancer treatment comprised an immunotherapy (e.g., with an anti-PD-1 antibody). In some aspects, the prior cancer treatment comprised a chemotherapy. In some aspects, the solid tumor has reoccurred. In some aspects, the solid tumor is metastatic. In some aspects, the solid tumor is not metastatic.

In some aspects, a subject that can be treated with the present disclosure comprises a nonhuman animal, such as a rat or a mouse. In some aspects, a subject that can be treated is a human.

In some aspects, the unit dose (e.g., for human use) of an IL-7 protein disclosed herein can be in the range of 0.001 mg/kg to 10 mg/kg. In certain aspects, the unit dose of an IL-7 protein is in the range of 0.01 mg/kg to 2 mg/kg. In some aspects, the unit dose is in the range of 0.02 mg/kg to 1 mg/kg. The unit dose can vary depending on the subject diseases for treatment and the presence of adverse effects.

In some aspects, an IL-7 protein disclosed herein can be administered to a subject at a weight-based dose. In certain aspects, an IL-7 protein can be administered at a weight-based dose between about 20 μg/kg and about 600 μg/kg. In certain aspects, an IL-7 protein of the present disclosure can be administered at a weight-based dose of about 20 μg/kg, about 60 μg/kg, about 120 μg/kg, about 240 μg/kg, about 360 μg/kg, about 480 μg/kg, or about 600 μg/kg.

In some aspects, an IL-7 protein disclosed herein can be administered to a subject at a dose greater than about 600 μg/kg. In certain aspects, an IL-7 protein is administered to a subject at a dose greater than about 600 μg/kg, greater than about 700 μg/kg, greater than about 800 μg/kg, greater than about 900 μg/kg, greater than about 1,000 μg/kg, greater than about 1,100 μg/kg, greater than about 1,200 μg/kg, greater than about 1,300 μg/kg, greater than about 1,400 μg/kg, greater than about 1,500 μg/kg, greater than about 1,600 μg/kg, greater than about 1,700 μg/kg, greater than about 1,800 μg/kg, greater than about 1,900 μg/kg, or greater than about 2,000 μg/kg.

In some aspects, an IL-7 protein of the present disclosure is administered at a dose of between 610 μg/kg and about 1,200 μg/kg, between 650 μg/kg and about 1,200 μg/kg, between about 700 μg/kg and about 1,200 μg/kg, between about 750 μg/kg and about 1,200 μg/kg, between about 800 μg/kg and about 1,200 μg/kg, between about 850 μg/kg and about 1,200 μg/kg, between about 900 μg/kg and about 1,200 μg/kg, between about 950 μg/kg and about 1,200 μg/kg, between about 1,000 μg/kg and about 1,200 μg/kg, between about 1,050 μg/kg and about 1,200 μg/kg, between about 1,100 μg/kg and about 1,200 μg/kg, between about 1,200 μg/kg and about 2,000 μg/kg, between about 1,300 μg/kg and about 2,000 μg/kg, between about 1,500 μg/kg and about 2,000 μg/kg, between about 1,700 μg/kg and about 2,000 μg/kg, between about 610 μg/kg and about 1,000 μg/kg, between about 650 μg/kg and about 1,000 μg/kg, between about 700 μg/kg and about 1,000 μg/kg, between about 750 μg/kg and about 1,000 μg/kg, between about 800 μg/kg and about 1,000 μg/kg, between about 850 μg/kg and about 1,000 μg/kg, between about 900 μg/kg and about 1,000 μg/kg, or between about 950 μg/kg and about 1,000 μg/kg.

In some aspects, an IL-7 protein of the present disclosure is administered at a dose of between 610 μg/kg and about 1,200 μg/kg. In certain aspects, an IL-7 protein is administered at a dose of between 650 μg/kg and about 1,200 μg/kg. In some aspects, an IL-7 protein is administered at a dose of between about 700 μg/kg and about 1,200 μg/kg. In certain aspects, an IL-7 protein is administered at a dose of between about 750 μg/kg and about 1,200 μg/kg. In certain aspects, an IL-7 protein is administered at a dose of between about 800 μg/kg and about 1,200 μg/kg. In some aspects, an IL-7 protein is administered at a dose of between about 850 μg/kg and about 1,200 μg/kg. In some aspects, an IL-7 protein is administered at a dose of between about 900 μg/kg and about 1,200 μg/kg. In certain aspects, an IL-7 protein is administered at a dose of between about 950 μg/kg and about 1,200 μg/kg. In some aspects, an IL-7 protein disclosed herein is administered at a dose of between about 1,000 μg/kg and about 1,200 μg/kg. In some aspects, an IL-7 protein is administered at a dose of between about 1,050 μg/kg and about 1,200 μg/kg. In some aspects, an IL-7 protein is administered at a dose of between about 1,100 μg/kg and about 1,200 μg/kg. In some aspects, an IL-7 protein is administered at a dose of between about 1,200 μg/kg and about 2,000 μg/kg. In certain aspects, an IL-7 protein is administered at a dose of between about 1,300 μg/kg and about 2,000 μg/kg. In some aspects, an IL-7 protein is administered at a dose of between about 1,500 μg/kg and about 2,000 μg/kg. In some aspects, an IL-7 protein is administered at a dose of between about 1,700 μg/kg and about 2,000 μg/kg. In certain aspects, an IL-7 protein is administered at a dose of between about 610 μg/kg and about 1,000 μg/kg. In some aspects, an IL-7 protein is administered at a dose of between about 650 μg/kg and about 1,000 μg/kg. In certain aspects, an IL-7 protein is administered at a dose of between about 700 μg/kg and about 1,000 μg/kg. In yet certain aspects, an IL-7 protein is administered at a dose of between about 750 μg/kg and about 1,000 μg/kg. In certain aspects, an IL-7 protein is administered at a dose of between about 800 μg/kg and about 1,000 μg/kg. In some aspects, an IL-7 protein is administered at a dose of between about 850 μg/kg and about 1,000 μg/kg. In some aspects, an IL-7 protein of the present disclosure is administered at a dose of between about 900 μg/kg and about 1,000 μg/kg. In some aspects, an IL-7 protein is administered at a dose of between about 950 μg/kg and about 1,000 μg/kg.

In some aspects, an IL-7 protein is administered at a dose of between about 700 μg/kg and about 900 μg/kg, between about 750 μg/kg and about 950 μg/kg, between about 700 μg/kg and about 850 μg/kg, between about 750 μg/kg and about 850 μg/kg, between about 700 μg/kg and about 800 μg/kg, between about 800 μg/kg and about 900 μg/kg, between about 750 μg/kg and about 850 μg/kg, or between about 850 μg/kg and about 950 μg/kg. In some aspects, an IL-7 protein is administered at a dose of between about 700 μg/kg and about 900 μg/kg. In certain aspects, an IL-7 protein is administered at a dose of between about 750 μg/kg and about 950 μg/kg. In certain aspects, an IL-7 protein is administered at a dose of between about 700 μg/kg and about 850 μg/kg. In some aspects, an IL-7 protein is administered at a dose of between about 750 μg/kg and about 850 μg/kg. In some aspects, an IL-7 protein is administered at a dose of between about 700 μg/kg and about 800 μg/kg. In some aspects, an IL-7 protein is administered at a dose of between about 800 μg/kg and about 900 μg/kg. In some aspects, an IL-7 protein is administered at a dose of between about 750 μg/kg and about 850 μg/kg. In certain aspects, an IL-7 protein is administered at a dose of between about 850 μg/kg and about 950 μg/kg.

In some aspects, an IL-7 protein is administered at a dose of about 650 μg/kg, about 680 μg/kg, about 700 μg/kg, about 720 μg/kg, about 740 μg/kg, about 750 μg/kg, about 760 μg/kg, about 780 μg/kg, about 800 μg/kg, about 820 μg/kg, about 840 μg/kg, about 850 μg/kg, about 860 μg/kg, about 880 μg/kg, about 900 μg/kg, about 920 μg/kg, about 940 μg/kg, about 950 μg/kg, about 960 μg/kg, about 980 μg/kg, about 1,000 μg/kg, about 1,020 μg/kg, about 1,020 μg/kg, about 1,040 μg/kg, about 1,060 μg/kg, about 1,080 μg/kg, about 1,100 μg/kg, about 1,120 μg/kg, about 1,140 μg/kg, about 1,160 μg/kg, about 1,180 μg/kg, about 1200 μg/kg, about 1,220 μg/kg, about 1,240 μg/kg, about 1,260 μg/kg, about 1,280 μg/kg, about 1,300 μg/kg, about 1,320 μg/kg, about 1,340 μg/kg, about 1,360 μg/kg, about 1,380 μg/kg, about 1,400 μg/kg, about 1,420 μg/kg, about 1,440 μg/kg, about 1,460 μg/kg, about 1,480 μg/kg, about 1,500 μg/kg, about 1,520 μg/kg, about 1,540 μg/kg, about 1,560 μg/kg, about 1,580 μg/kg, about 1,600 μg/kg, about 1,620 μg/kg, about 1,640 μg/kg, about 1,660 μg/kg, about 1,680 μg/kg, about 1,700 μg/kg, about 1,720 μg/kg, about 1,740 μg/kg, about 1,760 μg/kg, about 1,780 μg/kg, about 1,800 μg/kg, about 1,820 μg/kg, about 1,840 μg/kg, about 1,860 μg/kg, about 1,880 μg/kg, about 1,900 μg/kg, about 1,920 μg/kg, about 1,940 μg/kg, about 1,960 μg/kg, about 1,980 μg/kg, or about 2,000 μg/kg.

In some aspects, an IL-7 protein is administered at a dose of about 650 μg/kg. In some aspects, an IL-7 protein disclosed herein is administered at a dose of about 680 μg/kg. In some aspects, an IL-7 protein is administered at a dose of about 700 μg/kg. In some aspects, an IL-7 protein is administered at a dose of about 720 μg/kg. In certain aspects, an IL-7 protein is administered at a dose of about 740 μg/kg. In some aspects, an IL-7 protein is administered at a dose of about 750 μg/kg. In some aspects, an IL-7 protein is administered at a dose of about 760 μg/kg. In certain aspects, an IL-7 protein is administered at a dose of about 780 μg/kg. In some aspects, an IL-7 protein is administered at a dose of about 800 μg/kg. In certain aspects, an IL-7 protein is administered at a dose of about 820 μg/kg. In certain aspects, an IL-7 protein is administered at a dose of about 840 μg/kg. In some aspects, an IL-7 protein is administered at a dose of about 850 μg/kg. In certain aspects, an IL-7 protein is administered at a dose of about 860 μg/kg. In some aspects, an IL-7 protein is administered at a dose of about 880 μg/kg. In some aspects, an IL-7 protein is administered at a dose of about 900 μg/kg. In certain aspects, an IL-7 protein is administered at a dose of about 920 μg/kg. In some aspects, an IL-7 protein is administered at a dose of about 940 μg/kg. In certain aspects, an IL-7 protein is administered at a dose of about 950 μg/kg. In some aspects, an IL-7 protein is administered at a dose of about 960 μg/kg. In certain aspects, an IL-7 protein is administered at a dose of about 980 μg/kg. In some aspects, an IL-7 protein is administered at a dose of about 1,000 μg/kg. In certain aspects, an IL-7 protein is administered at a dose of about 1,020 μg/kg. In certain aspects, an IL-7 protein is administered at a dose of about 1,040 μg/kg. In some aspects, an IL-7 protein is administered at a dose of about 1,060 μg/kg. In some aspects, an IL-7 protein is administered at a dose of about 1,080 μg/kg. In some aspects, an IL-7 protein is administered at a dose of about 1,100 μg/kg. In certain aspects, an IL-7 protein is administered at a dose of about 1,120 μg/kg. In certain aspects, an IL-7 protein is administered at a dose of about 1,140 μg/kg. In some aspects, an IL-7 protein is administered at a dose of about 1,160 μg/kg. In some aspects, an IL-7 protein is administered at a dose of about 1,180 μg/kg. In certain aspects, an IL-7 protein is administered at a dose of about 1,200 μg/kg. In certain aspects, an IL-7 protein is administered at a dose of about 1,220 μg/kg. In certain aspects, an IL-7 protein is administered at a dose of about 1,240 μg/kg. In some aspects, an IL-7 protein is administered at a dose of about 1,260 μg/kg. In some aspects, an IL-7 protein is administered at a dose of about 1,280 μg/kg. In some aspects, an IL-7 protein is administered at a dose of about 1,300 μg/kg. In certain aspects, an IL-7 protein is administered at a dose of about 1,320 μg/kg. In certain aspects, an IL-7 protein is administered at a dose of about 1,340 μg/kg. In some aspects, an IL-7 protein is administered at a dose of about 1,360 μg/kg. In some aspects, an IL-7 protein is administered at a dose of about 1,380 μg/kg. In certain aspects, an IL-7 protein is administered at a dose of about 1,400 μg/kg. In certain aspects, an IL-7 protein is administered at a dose of about 1,420 μg/kg. In some aspects, an IL-7 protein is administered at a dose of about 1,440 μg/kg. In some aspects, an IL-7 protein is administered at a dose of about 1,460 μg/kg. In some aspects, an IL-7 protein is administered at a dose of about 1,480 μg/kg. In certain aspects, an IL-7 protein is administered at a dose of about 1,500 μg/kg. In certain aspects, an IL-7 protein is administered at a dose of about 1,520 μg/kg. In certain aspects, an IL-7 protein is administered at a dose of about 1,540 μg/kg. In some aspects, an IL-7 protein is administered at a dose of about 1,560 μg/kg. In some aspects, an IL-7 protein is administered at a dose of about 1,580 μg/kg. In some aspects, an IL-7 protein is administered at a dose of about 1,600 μg/kg. In certain aspects, an IL-7 protein is administered at a dose of about 1,620 μg/kg. In certain aspects, an IL-7 protein is administered at a dose of about 1,640 μg/kg. In some aspects, an IL-7 protein is administered at a dose of about 1,660 μg/kg. In some aspects, an IL-7 protein is administered at a dose of about 1,680 μg/kg. In certain aspects, an IL-7 protein is administered at a dose of about 1,700 μg/kg. In certain aspects, an IL-7 protein is administered at a dose of about 1,720 μg/kg. In certain aspects, an IL-7 protein is administered at a dose of about 1,740 μg/kg. In some aspects, an IL-7 protein is administered at a dose of about 1,760 μg/kg. In some aspects, an IL-7 protein is administered at a dose of about 1,780 μg/kg. In some aspects, an IL-7 protein is administered at a dose of about 1,800 μg/kg. In certain aspects, an IL-7 protein is administered at a dose of about 1,820 μg/kg. In certain aspects, an IL-7 protein is administered at a dose of about 1,840 μg/kg. In some aspects, an IL-7 protein is administered at a dose of about 1,860 μg/kg. In some aspects, an IL-7 protein is administered at a dose of about 1,880 μg/kg. In some aspects, an IL-7 protein is administered at a dose of about 1,900 μg/kg. In certain aspects, an IL-7 protein is administered at a dose of about 1,920 μg/kg. In certain aspects, an IL-7 protein is administered at a dose of about 1,940 μg/kg. In some aspects, an IL-7 protein is administered at a dose of about 1,960 μg/kg. In some aspects, an IL-7 protein is administered at a dose of about 1,980 μg/kg. In certain aspects, an IL-7 protein is administered at a dose of about 2,000 μg/kg.

In some aspects, an IL-7 protein can be administered at a flat dose. In certain aspects, an IL-7 protein can be administered at a flat dose of about 0.25 mg to about 9 mg. In some aspects, an IL-7 protein can be administered at a flat dose of about 0.25 mg, about 1 mg, about 3 mg, about 6 mg, or about 9 mg.

In some aspects, an IL-7 protein disclosed herein is administered to a subject at multiple doses (i.e., repeated administrations). In certain embodiments, an IL-7 protein is administered to the subject at least two times, at least three times, at least four times, at least five times, at least six times, at least seven times, at least eight times, at least nine times, or at least ten times or more. In other embodiments, a subject receives a single administration of the IL-7 protein (e.g., prior to, concurrently, or after an administration of a population of modified immune cells disclosed herein, e.g., CAR-bearing immune cells and/or transgenic TCR-bearing immune cells).

In some aspects, an IL-7 protein is administered at a dosing frequency of about once a week, about once in two weeks, about once in three weeks, about once in four weeks, about once in five weeks, about once in six weeks, about once in seven weeks, about once in eight weeks, about once in nine weeks, about once in 10 weeks, about once in 11 weeks, or about once in 12 weeks. In certain aspects, an IL-7 protein is administered at a dosing frequency of about once every 10 days, about once every 20 days, about once every 30 days, about once every 40 days, about once every 50 days, about once every 60 days, about once every 70 days, about once every 80 days, about once every 90 days, or about once every 100 days. In some aspects, the IL-7 protein is administered once in three weeks. In some aspects, the IL-7 protein is administered once a week. In some aspects, the IL-7 protein is administered once in two weeks. In certain aspects, the IL-7 protein is administered once in three weeks. In some aspects, the IL-7 protein is administered once in four weeks. In certain aspects, the IL-7 protein is administered once in six weeks. In certain aspects, the IL-7 protein is administered once in eight weeks. In some aspects, the IL-7 protein is administered once in nine weeks. In certain aspects, the IL-7 protein is administered once in 12 weeks. In some aspects, the IL-7 protein is administered once every 10 days. In certain aspects, the IL-7 protein is administered once every 20 days. In some aspects, the IL-7 protein is administered once every 30 days. In some aspects, the IL-7 protein is administered once every 40 days. In certain aspects, the IL-7 protein is administered once every 50 days. In some aspects, the IL-7 protein is administered once every 60 days. In certain aspects, the IL-7 protein is administered once every 70 days. In some aspects, the IL-7 protein is administered once every 80 days. In certain aspects, the IL-7 protein is administered once every 90 days. In some aspects, the IL-7 protein is administered once every 100 days.

In some aspects, the IL-7 protein is administered twice or more times in an amount of about 720 μg/kg at an interval of about 1 week, about 2 weeks, about 3 weeks, or about 4 weeks. In some aspects, the IL-7 protein is administered twice or more times in an amount of about 840 μg/kg at an interval of about 2 weeks, about 3 weeks, about 4 weeks, or about 5 weeks. In some aspects, the IL-7 protein is administered twice or more times in an amount of about 960 μg/kg at an interval of about 2 weeks, about 3 weeks, about 4 weeks, about 5 weeks, or about 6 weeks. In some aspects, the IL-7 protein is administered twice or more times in an amount of about 1200 μg/kg at an interval of about 3 weeks, about 4 weeks, about 5 weeks, about 6 weeks, about 7 weeks, or about 8 weeks. In some aspects, the IL-7 protein is administered twice or more times in an amount of about 1440 μg/kg at an interval of about 3 weeks, about 4 weeks, about 5 weeks, about 6 weeks, about 7 weeks, about 8 weeks, about 2 months, about 8 weeks, about 10 weeks, about 12 weeks, or about 3 months.

In some aspects, the IL-7 protein is administered at a dose of 60 μg/kg with a dosing frequency of once a week. In some aspects, the IL-7 protein is administered at a dose of 120 μg/kg with a dosing frequency of once a week. In some aspects, the IL-7 protein is administered at a dose of 240 μg/kg with a dosing frequency of once a week. In some aspects, the IL-7 protein is administered at a dose of 480 μg/kg with a dosing frequency of once a week. In some aspects, the IL-7 protein is administered at a dose of 720 μg/kg with a dosing frequency of once a week. In some aspects, the IL-7 protein is administered at a dose of 960 μg/kg with a dosing frequency of once a week. In some aspects, the IL-7 protein is administered at a dose of 1,200 μg/kg with a dosing frequency of once a week. In some aspects, the IL-7 protein is administered at a dose of 1,300 μg/kg with a dosing frequency of once a week. In some aspects, the IL-7 protein is administered at a dose of 1,400 μg/kg with a dosing frequency of once a week. In some aspects, the IL-7 protein is administered at a dose of 1,420 μg/kg with a dosing frequency of once a week. In certain aspects, the IL-7 protein is administered at a dose of 1,440 μg/kg with a dosing frequency of once a week. In certain aspects, the IL-7 protein is administered at a dose of 1,460 μg/kg with a dosing frequency of once a week. In certain aspects, the IL-7 protein is administered at a dose of 1,480 μg/kg with a dosing frequency of once a week. In some aspects, the IL-7 protein is administered at a dose of 1,500 μg/kg with a dosing frequency of once a week. In certain aspects, the IL-7 protein is administered at a dose of 1,600 μg/kg with a dosing frequency of once a week. In some aspects, the IL-7 protein is administered at a dose of 1,700 μg/kg with a dosing frequency of once a week. In some aspects, the IL-7 protein is administered at a dose of 2,000 μg/kg with a dosing frequency of once a week.

In some aspects, the IL-7 protein is administered at a dose of 60 μg/kg with a dosing frequency of once in two weeks. In some aspects, the IL-7 protein is administered at a dose of 120 μg/kg with a dosing frequency of once in two weeks. In some aspects, the IL-7 protein is administered at a dose of 240 μg/kg with a dosing frequency of once in two weeks. In some aspects, the IL-7 protein is administered at a dose of 480 μg/kg with a dosing frequency of once in two weeks. In some aspects, the IL-7 protein is administered at a dose of 720 μg/kg with a dosing frequency of once in two weeks. In some aspects, the IL-7 protein is administered at a dose of 960 μg/kg with a dosing frequency of once in two weeks. In some aspects, the IL-7 protein is administered at a dose of 1,200 μg/kg with a dosing frequency of once in two weeks. In some aspects, the IL-7 protein is administered at a dose of 1,300 μg/kg with a dosing frequency of once in two weeks. In some aspects, the IL-7 protein is administered at a dose of 1,400 μg/kg with a dosing frequency of once in two weeks. In some aspects, the IL-7 protein is administered at a dose of 1,420 μg/kg with a dosing frequency of once in two weeks. In certain aspects, the IL-7 protein is administered at a dose of 1,440 μg/kg with a dosing frequency of once in two weeks. In certain aspects, the IL-7 protein is administered at a dose of 1,460 μg/kg with a dosing frequency of once in two weeks. In certain aspects, the IL-7 protein is administered at a dose of 1,480 μg/kg with a dosing frequency of once in two weeks. In some aspects, the IL-7 protein is administered at a dose of 1,500 μg/kg with a dosing frequency of once in two weeks. In certain aspects, the IL-7 protein is administered at a dose of 1,600 μg/kg with a dosing frequency of once in two weeks. In some aspects, the IL-7 protein is administered at a dose of 1,700 μg/kg with a dosing frequency of once in two weeks. In some aspects, the IL-7 protein is administered at a dose of 2,000 μg/kg with a dosing frequency of once in two weeks.

In some aspects, the IL-7 protein is administered at a dose of 60 μg/kg with a dosing frequency of once in three weeks. In some aspects, the IL-7 protein is administered at a dose of 120 μg/kg with a dosing frequency of once in three weeks. In some aspects, the IL-7 protein is administered at a dose of 240 μg/kg with a dosing frequency of once in three weeks. In some aspects, the IL-7 protein is administered at a dose of 480 μg/kg with a dosing frequency of once in three weeks. In some aspects, the IL-7 protein is administered at a dose of 720 μg/kg with a dosing frequency of once in three weeks. In some aspects, the IL-7 protein is administered at a dose of 960 μg/kg with a dosing frequency of once in three weeks. In some aspects, the IL-7 protein is administered at a dose of 1,200 μg/kg with a dosing frequency of once in three weeks. In some aspects, the IL-7 protein is administered at a dose of 1,300 μg/kg with a dosing frequency of once in three weeks. In some aspects, the IL-7 protein is administered at a dose of 1,400 μg/kg with a dosing frequency of once in three weeks. In some aspects, the IL-7 protein is administered at a dose of 1,420 μg/kg with a dosing frequency of once in three weeks. In certain aspects, the IL-7 protein is administered at a dose of 1,440 μg/kg with a dosing frequency of once in three weeks. In certain aspects, the IL-7 protein is administered at a dose of 1,460 μg/kg with a dosing frequency of once in three weeks. In certain aspects, the IL-7 protein is administered at a dose of 1,480 μg/kg with a dosing frequency of once in three weeks. In some aspects, the IL-7 protein is administered at a dose of 1,500 μg/kg with a dosing frequency of once in three weeks. In certain aspects, the IL-7 protein is administered at a dose of 1,600 μg/kg with a dosing frequency of once in three weeks. In some aspects, the IL-7 protein is administered at a dose of 1,700 μg/kg with a dosing frequency of once in three weeks. In some aspects, the IL-7 protein is administered at a dose of 2,000 μg/kg with a dosing frequency of once in three weeks.

In some aspects, the IL-7 protein is administered at a dose of 60 μg/kg with a dosing frequency of once in four weeks. In some aspects, the IL-7 protein is administered at a dose of 120 μg/kg with a dosing frequency of once in four weeks. In some aspects, the IL-7 protein is administered at a dose of 240 μg/kg with a dosing frequency of once in four weeks. In some aspects, the IL-7 protein is administered at a dose of 480 μg/kg with a dosing frequency of once in four weeks. In some aspects, the IL-7 protein is administered at a dose of 720 μg/kg with a dosing frequency of once in four weeks. In some aspects, the IL-7 protein is administered at a dose of 960 μg/kg with a dosing frequency of once in four weeks. In some aspects, the IL-7 protein is administered at a dose of 1,200 μg/kg with a dosing frequency of once in four weeks. In some aspects, the IL-7 protein is administered at a dose of 1,300 μg/kg with a dosing frequency of once in four weeks. In some aspects, the IL-7 protein is administered at a dose of 1,400 μg/kg with a dosing frequency of once in four weeks. In some aspects, the IL-7 protein is administered at a dose of 1,420 μg/kg with a dosing frequency of once in four weeks. In certain aspects, the IL-7 protein is administered at a dose of 1,440 μg/kg with a dosing frequency of once in four weeks. In certain aspects, the IL-7 protein is administered at a dose of 1,460 μg/kg with a dosing frequency of once in four weeks. In certain aspects, the IL-7 protein is administered at a dose of 1,480 μg/kg with a dosing frequency of once in four weeks. In some aspects, the IL-7 protein is administered at a dose of 1,500 μg/kg with a dosing frequency of once in four weeks. In certain aspects, the IL-7 protein is administered at a dose of 1,600 μg/kg with a dosing frequency of once in four weeks. In some aspects, the IL-7 protein is administered at a dose of 1,700 μg/kg with a dosing frequency of once in four weeks. In some aspects, the IL-7 protein is administered at a dose of 2,000 μg/kg with a dosing frequency of once in four weeks.

In some aspects, the IL-7 protein is administered at a dose of 60 μg/kg with a dosing frequency of once in five weeks. In some aspects, the IL-7 protein is administered at a dose of 120 μg/kg with a dosing frequency of once in five weeks. In some aspects, the IL-7 protein is administered at a dose of 240 μg/kg with a dosing frequency of once in five weeks. In some aspects, the IL-7 protein is administered at a dose of 480 μg/kg with a dosing frequency of once in five weeks. In some aspects, the IL-7 protein is administered at a dose of 720 μg/kg with a dosing frequency of once in five weeks. In some aspects, the IL-7 protein is administered at a dose of 960 μg/kg with a dosing frequency of once in five weeks. In some aspects, the IL-7 protein is administered at a dose of 1,200 μg/kg with a dosing frequency of once in five weeks. In some aspects, the IL-7 protein is administered at a dose of 1,300 μg/kg with a dosing frequency of once in five weeks. In some aspects, the IL-7 protein is administered at a dose of 1,400 μg/kg with a dosing frequency of once in five weeks. In some aspects, the IL-7 protein is administered at a dose of 1,420 μg/kg with a dosing frequency of once in five weeks. In certain aspects, the IL-7 protein is administered at a dose of 1,440 μg/kg with a dosing frequency of once in five weeks. In certain aspects, the IL-7 protein is administered at a dose of 1,460 μg/kg with a dosing frequency of once in five weeks. In certain aspects, the IL-7 protein is administered at a dose of 1,480 μg/kg with a dosing frequency of once in five weeks. In some aspects, the IL-7 protein is administered at a dose of 1,500 μg/kg with a dosing frequency of once in five weeks. In certain aspects, the IL-7 protein is administered at a dose of 1,600 μg/kg with a dosing frequency of once in five weeks. In some aspects, the IL-7 protein is administered at a dose of 1,700 μg/kg with a dosing frequency of once in five weeks. In some aspects, the IL-7 protein is administered at a dose of 2,000 μg/kg with a dosing frequency of once in five weeks.

In some aspects, the IL-7 protein is administered at a dose of 60 μg/kg with a dosing frequency of once in six weeks. In some aspects, the IL-7 protein is administered at a dose of 120 μg/kg with a dosing frequency of once in six weeks. In some aspects, the IL-7 protein is administered at a dose of 240 μg/kg with a dosing frequency of once in six weeks. In some aspects, the IL-7 protein is administered at a dose of 480 μg/kg with a dosing frequency of once in six weeks. In some aspects, the IL-7 protein is administered at a dose of 720 μg/kg with a dosing frequency of once in six weeks. In some aspects, the IL-7 protein is administered at a dose of 960 μg/kg with a dosing frequency of once in six weeks. In some aspects, the IL-7 protein is administered at a dose of 1,200 μg/kg with a dosing frequency of once in six weeks. In some aspects, the IL-7 protein is administered at a dose of 1,300 μg/kg with a dosing frequency of once in six weeks. In some aspects, the IL-7 protein is administered at a dose of 1,400 μg/kg with a dosing frequency of once in six weeks. In some aspects, the IL-7 protein is administered at a dose of 1,420 μg/kg with a dosing frequency of once in six weeks. In certain aspects, the IL-7 protein is administered at a dose of 1,440 μg/kg with a dosing frequency of once in six weeks. In certain aspects, the IL-7 protein is administered at a dose of 1,460 μg/kg with a dosing frequency of once in six weeks. In certain aspects, the IL-7 protein is administered at a dose of 1,480 μg/kg with a dosing frequency of once in six weeks. In some aspects, the IL-7 protein is administered at a dose of 1,500 μg/kg with a dosing frequency of once in six weeks. In certain aspects, the IL-7 protein is administered at a dose of 1,600 μg/kg with a dosing frequency of once in six weeks. In some aspects, the IL-7 protein is administered at a dose of 1,700 μg/kg with a dosing frequency of once in six weeks. In some aspects, the IL-7 protein is administered at a dose of 2,000 μg/kg with a dosing frequency of once in six weeks.

In some aspects, the IL-7 protein is administered at a dose of 60 μg/kg with a dosing frequency of once in seven weeks. In some aspects, the IL-7 protein is administered at a dose of 120 μg/kg with a dosing frequency of once in seven weeks. In some aspects, the IL-7 protein is administered at a dose of 240 μg/kg with a dosing frequency of once in seven weeks. In some aspects, the IL-7 protein is administered at a dose of 480 μg/kg with a dosing frequency of once in seven weeks. In some aspects, the IL-7 protein is administered at a dose of 720 μg/kg with a dosing frequency of once in seven weeks. In some aspects, the IL-7 protein is administered at a dose of 960 μg/kg with a dosing frequency of once in seven weeks. In some aspects, the IL-7 protein is administered at a dose of 1,200 μg/kg with a dosing frequency of once in seven weeks. In some aspects, the IL-7 protein is administered at a dose of 1,300 μg/kg with a dosing frequency of once in seven weeks. In some aspects, the IL-7 protein is administered at a dose of 1,400 μg/kg with a dosing frequency of once in seven weeks. In some aspects, the IL-7 protein is administered at a dose of 1,420 μg/kg with a dosing frequency of once in seven weeks. In certain aspects, the IL-7 protein is administered at a dose of 1,440 μg/kg with a dosing frequency of once in seven weeks. In certain aspects, the IL-7 protein is administered at a dose of 1,460 μg/kg with a dosing frequency of once in seven weeks. In certain aspects, the IL-7 protein is administered at a dose of 1,480 μg/kg with a dosing frequency of once in seven weeks. In some aspects, the IL-7 protein is administered at a dose of 1,500 μg/kg with a dosing frequency of once in seven weeks. In certain aspects, the IL-7 protein is administered at a dose of 1,600 μg/kg with a dosing frequency of once in seven weeks. In some aspects, the IL-7 protein is administered at a dose of 1,700 μg/kg with a dosing frequency of once in seven weeks. In some aspects, the IL-7 protein is administered at a dose of 2,000 μg/kg with a dosing frequency of once in seven weeks.

In some aspects, the IL-7 protein is administered at a dose of 60 μg/kg with a dosing frequency of once in eight weeks. In some aspects, the IL-7 protein is administered at a dose of 120 μg/kg with a dosing frequency of once in eight weeks. In some aspects, the IL-7 protein is administered at a dose of 240 μg/kg with a dosing frequency of once in eight weeks. In some aspects, the IL-7 protein is administered at a dose of 480 μg/kg with a dosing frequency of once in eight weeks. In some aspects, the IL-7 protein is administered at a dose of 720 μg/kg with a dosing frequency of once in eight weeks. In some aspects, the IL-7 protein is administered at a dose of 960 μg/kg with a dosing frequency of once in eight weeks. In some aspects, the IL-7 protein is administered at a dose of 1,200 μg/kg with a dosing frequency of once in eight weeks. In some aspects, the IL-7 protein is administered at a dose of 1,300 μg/kg with a dosing frequency of once in eight weeks. In some aspects, the IL-7 protein is administered at a dose of 1,400 μg/kg with a dosing frequency of once in eight weeks. In some aspects, the IL-7 protein is administered at a dose of 1,420 μg/kg with a dosing frequency of once in eight weeks. In certain aspects, the IL-7 protein is administered at a dose of 1,440 μg/kg with a dosing frequency of once in eight weeks. In certain aspects, the IL-7 protein is administered at a dose of 1,460 μg/kg with a dosing frequency of once in eight weeks. In certain aspects, the IL-7 protein is administered at a dose of 1,480 μg/kg with a dosing frequency of once in eight weeks. In some aspects, the IL-7 protein is administered at a dose of 1,500 μg/kg with a dosing frequency of once in eight weeks. In certain aspects, the IL-7 protein is administered at a dose of 1,600 μg/kg with a dosing frequency of once in eight weeks. In some aspects, the IL-7 protein is administered at a dose of 1,700 μg/kg with a dosing frequency of once in eight weeks. In some aspects, the IL-7 protein is administered at a dose of 2,000 μg/kg with a dosing frequency of once in eight weeks.

In some aspects, the IL-7 protein is administered at a dose of 60 μg/kg with a dosing frequency of once in nine weeks. In some aspects, the IL-7 protein is administered at a dose of 120 μg/kg with a dosing frequency of once in nine weeks. In some aspects, the IL-7 protein is administered at a dose of 240 μg/kg with a dosing frequency of once in nine weeks. In some aspects, the IL-7 protein is administered at a dose of 480 μg/kg with a dosing frequency of once in nine weeks. In some aspects, the IL-7 protein is administered at a dose of 720 μg/kg with a dosing frequency of once in nine weeks. In some aspects, the IL-7 protein is administered at a dose of 960 μg/kg with a dosing frequency of once in nine weeks. In some aspects, the IL-7 protein is administered at a dose of 1,200 μg/kg with a dosing frequency of once in nine weeks. In some aspects, the IL-7 protein is administered at a dose of 1,300 μg/kg with a dosing frequency of once in three weeks. In some aspects, the IL-7 protein is administered at a dose of 1,400 μg/kg with a dosing frequency of once in three weeks. In some aspects, the IL-7 protein is administered at a dose of 1,420 μg/kg with a dosing frequency of once in three weeks. In certain aspects, the IL-7 protein is administered at a dose of 1,440 μg/kg with a dosing frequency of once in nine weeks. In certain aspects, the IL-7 protein is administered at a dose of 1,460 μg/kg with a dosing frequency of once in three weeks. In certain aspects, the IL-7 protein is administered at a dose of 1,480 μg/kg with a dosing frequency of once in three weeks. In some aspects, the IL-7 protein is administered at a dose of 1,500 μg/kg with a dosing frequency of once in three weeks. In certain aspects, the IL-7 protein is administered at a dose of 1,600 μg/kg with a dosing frequency of once in three weeks. In some aspects, the IL-7 protein is administered at a dose of 1,700 μg/kg with a dosing frequency of once in nine weeks. In some aspects, the IL-7 protein is administered at a dose of 2,000 μg/kg with a dosing frequency of once in nine weeks.

In some aspects, the IL-7 protein is administered at a dose of 60 μg/kg with a dosing frequency of once in 10 weeks. In some aspects, the IL-7 protein is administered at a dose of 120 μg/kg with a dosing frequency of once in 10 weeks. In some aspects, the IL-7 protein is administered at a dose of 240 μg/kg with a dosing frequency of once in 10 weeks. In some aspects, the IL-7 protein is administered at a dose of 480 μg/kg with a dosing frequency of once in 10 weeks. In some aspects, the IL-7 protein is administered at a dose of 720 μg/kg with a dosing frequency of once in 10 weeks. In some aspects, the IL-7 protein is administered at a dose of 960 μg/kg with a dosing frequency of once in 10 weeks. In some aspects, the IL-7 protein is administered at a dose of 1,200 μg/kg with a dosing frequency of once in 10 weeks. In some aspects, the IL-7 protein is administered at a dose of 1,300 μg/kg with a dosing frequency of once in 10 weeks. In some aspects, the IL-7 protein is administered at a dose of 1,400 μg/kg with a dosing frequency of once in 10 weeks. In some aspects, the IL-7 protein is administered at a dose of 1,420 μg/kg with a dosing frequency of once in 10 weeks. In certain aspects, the IL-7 protein is administered at a dose of 1,440 μg/kg with a dosing frequency of once in 10 weeks. In certain aspects, the IL-7 protein is administered at a dose of 1,460 μg/kg with a dosing frequency of once in 10 weeks. In certain aspects, the IL-7 protein is administered at a dose of 1,480 μg/kg with a dosing frequency of once in 10 weeks. In some aspects, the IL-7 protein is administered at a dose of 1,500 μg/kg with a dosing frequency of once in 10 weeks. In certain aspects, the IL-7 protein is administered at a dose of 1,600 μg/kg with a dosing frequency of once in 10 weeks. In some aspects, the IL-7 protein is administered at a dose of 1,700 μg/kg with a dosing frequency of once in 10 weeks. In some aspects, the IL-7 protein is administered at a dose of 2,000 μg/kg with a dosing frequency of once in 10 weeks.

In some aspects, the IL-7 protein is administered at a dose of 60 μg/kg with a dosing frequency of once in 11 weeks. In some aspects, the IL-7 protein is administered at a dose of 120 μg/kg with a dosing frequency of once in 11 weeks. In some aspects, the IL-7 protein is administered at a dose of 240 μg/kg with a dosing frequency of once in 11 weeks. In some aspects, the IL-7 protein is administered at a dose of 480 μg/kg with a dosing frequency of once in 11 weeks. In some aspects, the IL-7 protein is administered at a dose of 720 μg/kg with a dosing frequency of once in 11 weeks. In some aspects, the IL-7 protein is administered at a dose of 960 μg/kg with a dosing frequency of once in 11 weeks. In some aspects, the IL-7 protein is administered at a dose of 1,200 μg/kg with a dosing frequency of once in 11 weeks. In some aspects, the IL-7 protein is administered at a dose of 1,300 μg/kg with a dosing frequency of once in 11 weeks. In some aspects, the IL-7 protein is administered at a dose of 1,400 μg/kg with a dosing frequency of once in 11 weeks. In some aspects, the IL-7 protein is administered at a dose of 1,420 μg/kg with a dosing frequency of once in 11 weeks. In certain aspects, the IL-7 protein is administered at a dose of 1,440 μg/kg with a dosing frequency of once in 11 weeks. In certain aspects, the IL-7 protein is administered at a dose of 1,460 μg/kg with a dosing frequency of once in 11 weeks. In certain aspects, the IL-7 protein is administered at a dose of 1,480 μg/kg with a dosing frequency of once in 11 weeks. In some aspects, the IL-7 protein is administered at a dose of 1,500 μg/kg with a dosing frequency of once in 11 weeks. In certain aspects, the IL-7 protein is administered at a dose of 1,600 μg/kg with a dosing frequency of once in 11 weeks. In some aspects, the IL-7 protein is administered at a dose of 1,700 μg/kg with a dosing frequency of once in 11 weeks. In some aspects, the IL-7 protein is administered at a dose of 2,000 μg/kg with a dosing frequency of once in 11 weeks.

In some aspects, the IL-7 protein is administered at a dose of 60 μg/kg with a dosing frequency of once in 12 weeks. In some aspects, the IL-7 protein is administered at a dose of 120 μg/kg with a dosing frequency of once in 12 weeks. In some aspects, the IL-7 protein is administered at a dose of 240 μg/kg with a dosing frequency of once in 12 weeks. In some aspects, the IL-7 protein is administered at a dose of 480 μg/kg with a dosing frequency of once in 12 weeks. In some aspects, the IL-7 protein is administered at a dose of 720 μg/kg with a dosing frequency of once in 12 weeks. In some aspects, the IL-7 protein is administered at a dose of 960 μg/kg with a dosing frequency of once in 12 weeks. In some aspects, the IL-7 protein is administered at a dose of 1,200 μg/kg with a dosing frequency of once in 12 weeks. In some aspects, the IL-7 protein is administered at a dose of 1,300 μg/kg with a dosing frequency of once in 12 weeks. In some aspects, the IL-7 protein is administered at a dose of 1,400 μg/kg with a dosing frequency of once in 12 weeks. In some aspects, the IL-7 protein is administered at a dose of 1,420 μg/kg with a dosing frequency of once in 12 weeks. In certain aspects, the IL-7 protein is administered at a dose of 1,440 μg/kg with a dosing frequency of once in 12 weeks. In certain aspects, the IL-7 protein is administered at a dose of 1,460 μg/kg with a dosing frequency of once in 12 weeks. In certain aspects, the IL-7 protein is administered at a dose of 1,480 μg/kg with a dosing frequency of once in 12 weeks. In some aspects, the IL-7 protein is administered at a dose of 1,500 μg/kg with a dosing frequency of once in 12 weeks. In certain aspects, the IL-7 protein is administered at a dose of 1,600 μg/kg with a dosing frequency of once in 12 weeks. In some aspects, the IL-7 protein is administered at a dose of 1,700 μg/kg with a dosing frequency of once in 12 weeks. In some aspects, the IL-7 protein is administered at a dose of 2,000 μg/kg with a dosing frequency of once in 12 weeks.

In some aspects, the IL-7 protein is administered at a dose of 60 μg/kg with a dosing frequency of once every 10 days. In some aspects, the IL-7 protein is administered at a dose of 120 μg/kg with a dosing frequency of once every 10 days. In some aspects, the IL-7 protein is administered at a dose of 240 μg/kg with a dosing frequency of once every 10 days. In some aspects, the IL-7 protein is administered at a dose of 480 μg/kg with a dosing frequency of once every 10 days. In some aspects, the IL-7 protein is administered at a dose of 720 μg/kg with a dosing frequency of once every 10 days. In some aspects, the IL-7 protein is administered at a dose of 960 μg/kg with a dosing frequency of once every 10 days. In some aspects, the IL-7 protein is administered at a dose of 1,200 μg/kg with a dosing frequency of once every 10 days. In some aspects, the IL-7 protein is administered at a dose of 1,300 μg/kg with a dosing frequency of once every 10 days. In some aspects, the IL-7 protein is administered at a dose of 1,400 μg/kg with a dosing frequency of once every 10 days. In some aspects, the IL-7 protein is administered at a dose of 1,420 μg/kg with a dosing frequency of once every 10 days. In certain aspects, the IL-7 protein is administered at a dose of 1,440 μg/kg with a dosing frequency of once every 10 days. In certain aspects, the IL-7 protein is administered at a dose of 1,460 μg/kg with a dosing frequency of once every 10 days. In certain aspects, the IL-7 protein is administered at a dose of 1,480 μg/kg with a dosing frequency of once every 10 days. In some aspects, the IL-7 protein is administered at a dose of 1,500 μg/kg with a dosing frequency of once every 10 days. In certain aspects, the IL-7 protein is administered at a dose of 1,600 μg/kg with a dosing frequency of once every 10 days. In some aspects, the IL-7 protein is administered at a dose of 1,700 μg/kg with a dosing frequency of once every 10 days. In some aspects, the IL-7 protein is administered at a dose of 2,000 μg/kg with a dosing frequency of once every 10 days.

In some aspects, the IL-7 protein is administered at a dose of 60 μg/kg with a dosing frequency of once every 20 days. In some aspects, the IL-7 protein is administered at a dose of 120 μg/kg with a dosing frequency of once every 20 days. In some aspects, the IL-7 protein is administered at a dose of 240 μg/kg with a dosing frequency of once every 20 days. In some aspects, the IL-7 protein is administered at a dose of 480 μg/kg with a dosing frequency of once every 20 days. In some aspects, the IL-7 protein is administered at a dose of 720 μg/kg with a dosing frequency of once every 20 days. In some aspects, the IL-7 protein is administered at a dose of 960 μg/kg with a dosing frequency of once every 20 days. In some aspects, the IL-7 protein is administered at a dose of 1,200 μg/kg with a dosing frequency of once every 20 days. In some aspects, the IL-7 protein is administered at a dose of 1,300 μg/kg with a dosing frequency of once every 20 days. In some aspects, the IL-7 protein is administered at a dose of 1,400 μg/kg with a dosing frequency of once every 20 days. In some aspects, the IL-7 protein is administered at a dose of 1,420 μg/kg with a dosing frequency of once every 20 days. In certain aspects, the IL-7 protein is administered at a dose of 1,440 μg/kg with a dosing frequency of once every 20 days. In certain aspects, the IL-7 protein is administered at a dose of 1,460 μg/kg with a dosing frequency of once every 20 days. In certain aspects, the IL-7 protein is administered at a dose of 1,480 μg/kg with a dosing frequency of once every 20 days. In some aspects, the IL-7 protein is administered at a dose of 1,500 μg/kg with a dosing frequency of once every 20 days. In certain aspects, the IL-7 protein is administered at a dose of 1,600 μg/kg with a dosing frequency of once every 20 days. In some aspects, the IL-7 protein is administered at a dose of 1,700 μg/kg with a dosing frequency of once every 20 days. In some aspects, the IL-7 protein is administered at a dose of 2,000 μg/kg with a dosing frequency of once every 20 days.

In some aspects, the IL-7 protein is administered at a dose of 60 μg/kg with a dosing frequency of once every 30 days. In some aspects, the IL-7 protein is administered at a dose of 120 μg/kg with a dosing frequency of once every 30 days. In some aspects, the IL-7 protein is administered at a dose of 240 μg/kg with a dosing frequency of once every 30 days. In some aspects, the IL-7 protein is administered at a dose of 480 μg/kg with a dosing frequency of once every 30 days. In some aspects, the IL-7 protein is administered at a dose of 720 μg/kg with a dosing frequency of once every 30 days. In some aspects, the IL-7 protein is administered at a dose of 960 μg/kg with a dosing frequency of once every 30 days. In some aspects, the IL-7 protein is administered at a dose of 1,200 μg/kg with a dosing frequency of once every 30 days. In some aspects, the IL-7 protein is administered at a dose of 1,300 μg/kg with a dosing frequency of once every 30 days. In some aspects, the IL-7 protein is administered at a dose of 1,400 μg/kg with a dosing frequency of once every 30 days. In some aspects, the IL-7 protein is administered at a dose of 1,420 μg/kg with a dosing frequency of once every 30 days. In certain aspects, the IL-7 protein is administered at a dose of 1,440 μg/kg with a dosing frequency of once every 30 days. In certain aspects, the IL-7 protein is administered at a dose of 1,460 μg/kg with a dosing frequency of once every 30 days. In certain aspects, the IL-7 protein is administered at a dose of 1,480 μg/kg with a dosing frequency of once every 30 days. In some aspects, the IL-7 protein is administered at a dose of 1,500 μg/kg with a dosing frequency of once every 30 days. In certain aspects, the IL-7 protein is administered at a dose of 1,600 μg/kg with a dosing frequency of once every 30 days. In some aspects, the IL-7 protein is administered at a dose of 1,700 μg/kg with a dosing frequency of once every 30 days. In some aspects, the IL-7 protein is administered at a dose of 2,000 μg/kg with a dosing frequency of once every 30 days.

In some aspects, the IL-7 protein is administered at a dose of 60 μg/kg with a dosing frequency of once every 40 days. In some aspects, the IL-7 protein is administered at a dose of 120 μg/kg with a dosing frequency of once every 40 days. In some aspects, the IL-7 protein is administered at a dose of 240 μg/kg with a dosing frequency of once every 40 days. In some aspects, the IL-7 protein is administered at a dose of 480 μg/kg with a dosing frequency of once every 40 days. In some aspects, the IL-7 protein is administered at a dose of 720 μg/kg with a dosing frequency of once every 40 days. In some aspects, the IL-7 protein is administered at a dose of 960 μg/kg with a dosing frequency of once every 40 days. In some aspects, the IL-7 protein is administered at a dose of 1,200 μg/kg with a dosing frequency of once every 40 days. In some aspects, the IL-7 protein is administered at a dose of 1,300 μg/kg with a dosing frequency of once every 40 days. In some aspects, the IL-7 protein is administered at a dose of 1,400 μg/kg with a dosing frequency of once every 40 days. In some aspects, the IL-7 protein is administered at a dose of 1,420 μg/kg with a dosing frequency of once every 40 days. In certain aspects, the IL-7 protein is administered at a dose of 1,440 μg/kg with a dosing frequency of once every 40 days. In certain aspects, the IL-7 protein is administered at a dose of 1,460 μg/kg with a dosing frequency of once every 40 days. In certain aspects, the IL-7 protein is administered at a dose of 1,480 μg/kg with a dosing frequency of once every 40 days. In some aspects, the IL-7 protein is administered at a dose of 1,500 μg/kg with a dosing frequency of once every 40 days. In certain aspects, the IL-7 protein is administered at a dose of 1,600 μg/kg with a dosing frequency of once every 40 days. In some aspects, the IL-7 protein is administered at a dose of 1,700 μg/kg with a dosing frequency of once every 40 days. In some aspects, the IL-7 protein is administered at a dose of 2,000 μg/kg with a dosing frequency of once every 40 days.

In some aspects, the IL-7 protein is administered at a dose of 60 μg/kg with a dosing frequency of once every 50 days. In some aspects, the IL-7 protein is administered at a dose of 120 μg/kg with a dosing frequency of once every 50 days. In some aspects, the IL-7 protein is administered at a dose of 240 μg/kg with a dosing frequency of once every 50 days. In some aspects, the IL-7 protein is administered at a dose of 480 μg/kg with a dosing frequency of once every 50 days. In some aspects, the IL-7 protein is administered at a dose of 720 μg/kg with a dosing frequency of once every 50 days. In some aspects, the IL-7 protein is administered at a dose of 960 μg/kg with a dosing frequency of once every 50 days. In some aspects, the IL-7 protein is administered at a dose of 1,200 μg/kg with a dosing frequency of once every 50 days. In some aspects, the IL-7 protein is administered at a dose of 1,300 μg/kg with a dosing frequency of once every 50 days. In some aspects, the IL-7 protein is administered at a dose of 1,400 μg/kg with a dosing frequency of once every 50 days. In some aspects, the IL-7 protein is administered at a dose of 1,420 μg/kg with a dosing frequency of once every 50 days. In certain aspects, the IL-7 protein is administered at a dose of 1,440 μg/kg with a dosing frequency of once every 50 days. In certain aspects, the IL-7 protein is administered at a dose of 1,460 μg/kg with a dosing frequency of once every 50 days. In certain aspects, the IL-7 protein is administered at a dose of 1,480 μg/kg with a dosing frequency of once every 50 days. In some aspects, the IL-7 protein is administered at a dose of 1,500 μg/kg with a dosing frequency of once every 50 days. In certain aspects, the IL-7 protein is administered at a dose of 1,600 μg/kg with a dosing frequency of once every 50 days. In some aspects, the IL-7 protein is administered at a dose of 1,700 μg/kg with a dosing frequency of once every 50 days. In some aspects, the IL-7 protein is administered at a dose of 2,000 μg/kg with a dosing frequency of once every 50 days.

In some aspects, the IL-7 protein is administered at a dose of 60 μg/kg with a dosing frequency of once every 60 days. In some aspects, the IL-7 protein is administered at a dose of 120 μg/kg with a dosing frequency of once every 60 days. In some aspects, the IL-7 protein is administered at a dose of 240 μg/kg with a dosing frequency of once every 60 days. In some aspects, the IL-7 protein is administered at a dose of 480 μg/kg with a dosing frequency of once every 60 days. In some aspects, the IL-7 protein is administered at a dose of 720 μg/kg with a dosing frequency of once every 60 days. In some aspects, the IL-7 protein is administered at a dose of 960 μg/kg with a dosing frequency of once every 60 days. In some aspects, the IL-7 protein is administered at a dose of 1,200 μg/kg with a dosing frequency of once every 60 days. In some aspects, the IL-7 protein is administered at a dose of 1,300 μg/kg with a dosing frequency of once every 60 days. In some aspects, the IL-7 protein is administered at a dose of 1,400 μg/kg with a dosing frequency of once every 60 days. In some aspects, the IL-7 protein is administered at a dose of 1,420 μg/kg with a dosing frequency of once every 60 days. In certain aspects, the IL-7 protein is administered at a dose of 1,440 μg/kg with a dosing frequency of once every 60 days. In certain aspects, the IL-7 protein is administered at a dose of 1,460 μg/kg with a dosing frequency of once every 60 days. In certain aspects, the IL-7 protein is administered at a dose of 1,480 μg/kg with a dosing frequency of once every 60 days. In some aspects, the IL-7 protein is administered at a dose of 1,500 μg/kg with a dosing frequency of once every 60 days. In certain aspects, the IL-7 protein is administered at a dose of 1,600 μg/kg with a dosing frequency of once every 60 days. In some aspects, the IL-7 protein is administered at a dose of 1,700 μg/kg with a dosing frequency of once every 60 days. In some aspects, the IL-7 protein is administered at a dose of 2,000 μg/kg with a dosing frequency of once every 60 days.

In some aspects, the IL-7 protein is administered at a dose of 60 μg/kg with a dosing frequency of once every 70 days. In some aspects, the IL-7 protein is administered at a dose of 120 μg/kg with a dosing frequency of once every 70 days. In some aspects, the IL-7 protein is administered at a dose of 240 μg/kg with a dosing frequency of once every 70 days. In some aspects, the IL-7 protein is administered at a dose of 480 μg/kg with a dosing frequency of once every 70 days. In some aspects, the IL-7 protein is administered at a dose of 720 μg/kg with a dosing frequency of once every 70 days. In some aspects, the IL-7 protein is administered at a dose of 960 μg/kg with a dosing frequency of once every 70 days. In some aspects, the IL-7 protein is administered at a dose of 1,200 μg/kg with a dosing frequency of once every 70 days. In some aspects, the IL-7 protein is administered at a dose of 1,300 μg/kg with a dosing frequency of once every 70 days. In some aspects, the IL-7 protein is administered at a dose of 1,400 μg/kg with a dosing frequency of once every 70 days. In some aspects, the IL-7 protein is administered at a dose of 1,420 μg/kg with a dosing frequency of once every 70 days. In certain aspects, the IL-7 protein is administered at a dose of 1,440 μg/kg with a dosing frequency of once every 70 days. In certain aspects, the IL-7 protein is administered at a dose of 1,460 μg/kg with a dosing frequency of once every 70 days. In certain aspects, the IL-7 protein is administered at a dose of 1,480 μg/kg with a dosing frequency of once every 70 days. In some aspects, the IL-7 protein is administered at a dose of 1,500 μg/kg with a dosing frequency of once every 70 days. In certain aspects, the IL-7 protein is administered at a dose of 1,600 μg/kg with a dosing frequency of once every 70 days. In some aspects, the IL-7 protein is administered at a dose of 1,700 μg/kg with a dosing frequency of once every 70 days. In some aspects, the IL-7 protein is administered at a dose of 2,000 μg/kg with a dosing frequency of once every 70 days.

In some aspects, the IL-7 protein is administered at a dose of 60 μg/kg with a dosing frequency of once every 80 days. In some aspects, the IL-7 protein is administered at a dose of 120 μg/kg with a dosing frequency of once every 80 days. In some aspects, the IL-7 protein is administered at a dose of 240 μg/kg with a dosing frequency of once every 80 days. In some aspects, the IL-7 protein is administered at a dose of 480 μg/kg with a dosing frequency of once every 80 days. In some aspects, the IL-7 protein is administered at a dose of 720 μg/kg with a dosing frequency of once every 80 days. In some aspects, the IL-7 protein is administered at a dose of 960 μg/kg with a dosing frequency of once every 80 days. In some aspects, the IL-7 protein is administered at a dose of 1,200 μg/kg with a dosing frequency of once every 80 days. In some aspects, the IL-7 protein is administered at a dose of 1,300 μg/kg with a dosing frequency of once every 80 days. In some aspects, the IL-7 protein is administered at a dose of 1,400 μg/kg with a dosing frequency of once every 80 days. In some aspects, the IL-7 protein is administered at a dose of 1,420 μg/kg with a dosing frequency of once every 80 days. In certain aspects, the IL-7 protein is administered at a dose of 1,440 μg/kg with a dosing frequency of once every 80 days. In certain aspects, the IL-7 protein is administered at a dose of 1,460 μg/kg with a dosing frequency of once every 80 days. In certain aspects, the IL-7 protein is administered at a dose of 1,480 μg/kg with a dosing frequency of once every 80 days. In some aspects, the IL-7 protein is administered at a dose of 1,500 μg/kg with a dosing frequency of once every 80 days. In certain aspects, the IL-7 protein is administered at a dose of 1,600 μg/kg with a dosing frequency of once every 80 days. In some aspects, the IL-7 protein is administered at a dose of 1,700 μg/kg with a dosing frequency of once every 80 days. In some aspects, the IL-7 protein is administered at a dose of 2,000 μg/kg with a dosing frequency of once every 80 days.

In some aspects, the IL-7 protein is administered at a dose of 60 μg/kg with a dosing frequency of once every 90 days. In some aspects, the IL-7 protein is administered at a dose of 120 μg/kg with a dosing frequency of once every 90 days. In some aspects, the IL-7 protein is administered at a dose of 240 μg/kg with a dosing frequency of once every 90 days. In some aspects, the IL-7 protein is administered at a dose of 480 μg/kg with a dosing frequency of once every 90 days. In some aspects, the IL-7 protein is administered at a dose of 720 μg/kg with a dosing frequency of once every 90 days. In some aspects, the IL-7 protein is administered at a dose of 960 μg/kg with a dosing frequency of once every 90 days. In some aspects, the IL-7 protein is administered at a dose of 1,200 μg/kg with a dosing frequency of once every 90 days. In some aspects, the IL-7 protein is administered at a dose of 1,300 μg/kg with a dosing frequency of once every 90 days. In some aspects, the IL-7 protein is administered at a dose of 1,400 μg/kg with a dosing frequency of once every 90 days. In some aspects, the IL-7 protein is administered at a dose of 1,420 μg/kg with a dosing frequency of once every 90 days. In certain aspects, the IL-7 protein is administered at a dose of 1,440 μg/kg with a dosing frequency of once every 90 days. In certain aspects, the IL-7 protein is administered at a dose of 1,460 μg/kg with a dosing frequency of once every 90 days. In certain aspects, the IL-7 protein is administered at a dose of 1,480 μg/kg with a dosing frequency of once every 90 days. In some aspects, the IL-7 protein is administered at a dose of 1,500 μg/kg with a dosing frequency of once every 90 days. In certain aspects, the IL-7 protein is administered at a dose of 1,600 μg/kg with a dosing frequency of once every 90 days. In some aspects, the IL-7 protein is administered at a dose of 1,700 μg/kg with a dosing frequency of once every 90 days. In some aspects, the IL-7 protein is administered at a dose of 2,000 μg/kg with a dosing frequency of once every 90 days.

In some aspects, the IL-7 protein is administered at a dose of 60 μg/kg with a dosing frequency of once every 100 days. In some aspects, the IL-7 protein is administered at a dose of 120 μg/kg with a dosing frequency of once every 100 days. In some aspects, the IL-7 protein is administered at a dose of 240 μg/kg with a dosing frequency of once every 100 days. In some aspects, the IL-7 protein is administered at a dose of 480 μg/kg with a dosing frequency of once every 100 days. In some aspects, the IL-7 protein is administered at a dose of 720 μg/kg with a dosing frequency of once every 100 days. In some aspects, the IL-7 protein is administered at a dose of 960 μg/kg with a dosing frequency of once every 100 days. In some aspects, the IL-7 protein is administered at a dose of 1,200 μg/kg with a dosing frequency of once every 100 days. In some aspects, the IL-7 protein is administered at a dose of 1,300 μg/kg with a dosing frequency of once every 100 days. In some aspects, the IL-7 protein is administered at a dose of 1,400 μg/kg with a dosing frequency of once every 100 days. In some aspects, the IL-7 protein is administered at a dose of 1,420 μg/kg with a dosing frequency of once every 100 days. In certain aspects, the IL-7 protein is administered at a dose of 1,440 μg/kg with a dosing frequency of once every 100 days. In certain aspects, the IL-7 protein is administered at a dose of 1,460 μg/kg with a dosing frequency of once every 100 days. In certain aspects, the IL-7 protein is administered at a dose of 1,480 μg/kg with a dosing frequency of once every 100 days. In some aspects, the IL-7 protein is administered at a dose of 1,500 μg/kg with a dosing frequency of once every 100 days. In certain aspects, the IL-7 protein is administered at a dose of 1,600 μg/kg with a dosing frequency of once every 100 days. In some aspects, the IL-7 protein is administered at a dose of 1,700 μg/kg with a dosing frequency of once every 100 days. In some aspects, the IL-7 protein is administered at a dose of 2,000 μg/kg with a dosing frequency of once every 100 days.

In some aspects, administering an IL-7 protein disclosed herein in combination with a population of modified immune cells (e.g., CAR-bearing immune cells and/or transgenic TCR-bearing immune cells) can decrease the dose of the population of modified immune cells (e.g., CAR-bearing immune cells and/or transgenic TCR-bearing immune cells) necessary to maintain clinical efficacy (e.g., dose necessary to reduce tumor volume and/or increase the duration of survival of the subject). Accordingly, in some aspects, the dose of the population of CAR-bearing immune cells is reduced by at least about 5%, at least about 10%, at least about 20%, at least about 30%, at least about 40%, at least about 50%, at least about 60%, at least about 70%, at least about 80%, or at least about 90% or more compared to a reference dose (e.g., dose of the population of CAR-bearing immune cells when administered alone or in combination with an agent other than the IL-7 protein of the present disclosure). In some aspects, the dose of the population of transgenic TCR-bearing immune cells is reduced by at least about 5%, at least about 10%, at least about 20%, at least about 30%, at least about 40%, at least about 50%, at least about 60%, at least about 70%, at least about 80%, or at least about 90% or more compared to a reference dose (e.g., dose of the population of transgenic TCR-bearing immune cells when administered alone or in combination with an agent other than the IL-7 protein of the present disclosure).

In some aspects, a dose of the population of CAR-bearing immune cells comprises less than about 10,000,000 CAR-bearing immune cells per kilogram of the subject's body weight. In some aspects, a dose of the population of CAR-bearing immune cells comprises less than about 1,000,000 CAR-bearing immune cells per kilogram of the subject's body weight. In some aspects, a dose of the population of CAR-bearing immune cells comprises less than about 100,000 CAR-bearing immune cells per kilogram of the subject's body weight. In some aspects, a dose of the population of CAR-bearing immune cells comprises less than about 50,000 CAR-bearing immune cells per kilogram of the subject's body weight. In some aspects, a dose of the population of CAR-bearing immune cells comprises less than about 10,000 CAR-bearing immune cells per kilogram of the subject's body weight. In certain aspects, a dose of the population of CAR-bearing immune cells comprises less than about 5,000 CAR-bearing immune cells per kilogram of the subject's body weight. In some aspects, a dose of the population of CAR-bearing immune cells comprises less than about 2,500 CAR-bearing immune cells per kilogram of the subject's body weight. In some aspects, a dose of the population of CAR-bearing immune cells comprises less than about 1,000 CAR-bearing immune cells per kilogram of the subject's body weight.

In some aspects, a dose of the population of transgenic TCR-bearing immune cells comprises less than about 10,000,000 transgenic TCR-bearing immune cells per kilogram of the subject's body weight. In some aspects, a dose of the population of transgenic TCR-bearing immune cells comprises less than about 1,000,000 transgenic TCR-bearing immune cells per kilogram of the subject's body weight. In some aspects, a dose of the population of transgenic TCR-bearing immune cells comprises less than about 10,000 transgenic TCR-bearing immune cells per kilogram of the subject's body weight. In some aspects, a dose of the population of transgenic TCR-bearing immune cells comprises less than about 50,000 transgenic TCR-bearing immune cells per kilogram of the subject's body weight. In some aspects, a dose of the population of transgenic TCR-bearing immune cells comprises less than about 10,000 transgenic TCR-bearing immune cells per kilogram of the subject's body weight. In certain aspects, a dose of the population of transgenic TCR-bearing immune cells comprises less than about 5,000 transgenic TCR-bearing immune cells per kilogram of the subject's body weight. In some aspects, a dose of the population of transgenic TCR-bearing immune cells comprises less than about 2,500 transgenic TCR-bearing immune cells per kilogram of the subject's body weight. In some aspects, a dose of the population of transgenic TCR-bearing immune cells comprises less than about 1,000 transgenic TCR-bearing immune cells per kilogram of the subject's body weight.

An IL-7 protein and population of modified immune cells (e.g., CAR-bearing immune cells and/or transgenic TCR-bearing immune cells) disclosed herein can be administered to a subject having a solid tumor by any relevant route of administration. In some aspects, the IL-7 protein is administered to the subject parenthetically, intramuscularly, subcutaneously, ophthalmic, intravenously, intraperitoneally, intradermally, intraorbitally, intracerebrally, intracranially, intraspinally, intraventricular, intrathecally, intracistemally, intracapsularly, or intratumorally. In some aspects, the population of modified immune cells (e.g., CAR-bearing immune cells and/or transgenic TCR-bearing immune cells) is administered intravenously.

In some aspects, methods disclosed herein (e.g., administering an IL-7 protein in combination with a population of modified immune cells, e.g., CAR-bearing immune cells and/or transgenic TCR-bearing immune cells) can be used in combination with one or more additional therapeutic agent (e.g., anti-cancer and/or immunomodulating agents). Such agents can include, for example, chemotherapy drugs, small molecule drugs, or antibodies that stimulate the immune response to a given cancer. In some aspects, the methods described herein are used in combination with a standard of care treatment (e.g., surgery, radiation, and chemotherapy). Methods described herein can also be used as a maintenance therapy, e.g., a therapy that is intended to prevent the occurrence or recurrence of solid tumors.

In some aspects, an additional therapeutic agent that can be used with the present disclosure comprises an immune checkpoint inhibitor (i.e., blocks signaling through the particular immune checkpoint pathway). Non-limiting examples of immune checkpoint inhibitors that can be used in the present methods comprise a CTLA-4 antagonist (e.g., anti-CTLA-4 antibody), PD-1 antagonist (e.g., anti-PD-1 antibody, anti-PD-L1 antibody), TIM-3 antagonist (e.g., anti-TIM-3 antibody), or combinations thereof.

In some aspects, the additional therapeutic agent comprises an immune checkpoint activator (i.e., promotes signaling through the particular immune checkpoint pathway). In certain aspects, the immune checkpoint activator comprises OX40 agonist (e.g., anti-OX40 antibody), LAG-3 agonist (e.g. anti-LAG-3 antibody), 4-1BB (CD137) agonist (e.g., anti-CD137 antibody), GITR agonist (e.g., anti-GITR antibody), or any combination thereof.

In some aspects, a subject having a solid tumor treated with the combination therapy disclosed herein (i.e., IL-7 protein in combination with a population of modified immune cells, e.g., CAR-bearing immune cells and/or transgenic TCR-bearing immune cells) can be further treated with a lymphocyte depleting agent. Non-limiting examples of lymphocyte depleting agents include antibodies (e.g., THYMOGLOBULIN®, ATGAM®, CAMPATH®) and chemotherapy agents (e.g., fludarabine)(FLUDARA® and cyclophosphamide)(CYTOXAN®). In some aspects, the subject is further treated with a kinase inhibitor (e.g., dasatinib (SPRYCEL®). In certain aspects, the kinase inhibitor can be used to reversibly block CAR-T cell function (e.g., to mitigate cytokine release syndrome).

In some aspects, an IL-7 protein (e.g., those disclosed herein) and a population of modified immune cells (e.g., CAR-bearing immune cells and/or transgenic TCR-bearing immune cells) can be administered concurrently as a single composition in a pharmaceutically acceptable carrier. In some aspects, the IL-7 protein and the population of modified immune cells (e.g., CAR-bearing immune cells and/or transgenic TCR-bearing immune cells) can be administered concurrently as separate compositions. In some aspects, the IL-7 protein and the population of modified immune cells (e.g., CAR-bearing immune cells and/or transgenic TCR-bearing immune cells) can be administered sequentially. For instances, in certain aspects, the IL-7 protein is administered to the subject prior to administering the population of modified immune cells (e.g., CAR-bearing immune cells and/or transgenic TCR-bearing immune cells). In certain aspects, the IL-7 protein is administered to the subject after administering the population of modified immune cells (e.g., CAR-bearing immune cells and/or transgenic TCR-bearing immune cells).

### III. IL-7 Proteins Useful for the Disclosure

Disclosed herein are IL-7 proteins that can be used in combination with a population of modified immune cells (e.g., CAR-bearing immune cells and/or transgenic TCR-bearing immune cells), e.g., to treat a solid tumor. In some aspects, IL-7 protein useful for the present uses can be wild-type IL-7 or modified IL-7 (i.e., not wild-type IL-7 protein) (e.g., IL-7 variant, IL-7 functional fragment, IL-7 derivative, or any combination thereof, e.g., fusion protein, chimeric protein, etc.) as long as the IL-7 protein contains one or more biological activities of IL-7, e.g., capable of binding to IL-7R, e.g., inducing early T-cell development, promoting T-cell homeostasis. See ElKassar and Gress. J Immunotoxicol. 2010 March; 7(1): 1-7. In some aspects, an IL-7 protein of the present disclosure is not a wild-type IL-7 protein (i.e., comprises one or more modifications). Non-limiting examples of such modifications can include an oligopeptide and/or a half-life extending moiety. See WO 2016/200219, which is herein incorporated by reference in its entirety.

IL-7 binds to its receptor which is composed of the two chains IL-7Ra (CD127), shared with the thymic stromal lymphopoietin (TSLP) (Ziegler and Liu, 2006), and the common γ chain (CD132) for IL-2, IL-15, IL-9 and IL-21. Whereas γc is expressed by most hematopoietic cells, IL-7Rα is nearly exclusively expressed on lymphoid cells. After binding to its receptor, IL-7 signals through two different pathways: Jak-Stat (Janus kinase-Signal transducer and activator of transcription) and PI3K/Akt responsible for differentiation and survival, respectively. The absence of IL-7 signaling is responsible for a reduced thymic cellularity as observed in mice that have received an anti-IL-7 neutralizing monoclonal antibody (MAb); Grabstein et al., 1993), in IL-7−/− (von Freeden-Jeffry et al., 1995), IL-7Rα−/− (Peschon et al., 1994; Maki et al., 1996), γc−/− (Malissen et al., 1997), and Jak3−/− mice (Park et al., 1995). In the absence of IL-7 signaling, mice lack T-, B-, and NK-T cells. IL-7α−/− mice (Peschon et al., 1994) have a similar but more severe phenotype than IL-7−/− mice (von Freeden-Jeffry et al., 1995), possibly because TSLP signaling is also abrogated in IL-7α−/− mice. IL-7 is required for the development of γδ cells (Maki et al., 1996) and NK-T cells (Boesteanu et al., 1997).

In some aspects, an IL-7 protein useful for the present disclosure comprises an amino acid sequence as set forth in any one of SEQ ID NOs: 1 to 6. In some aspects, the IL-7 protein comprises an amino acid sequence having a sequence identity of about 70%, about 75%, about 80%, about 85%, about 90%, about 91%, about 92%, about 93%, about 94%, about 95%, about 96%, about 97%, about 98%, or about 99% or higher, to a sequence of SEQ ID NOS: 1 to 6.

In some aspects, the IL-7 protein includes a modified IL-7 or a fragment thereof, wherein the modified IL-7 or the fragment retains one or more biological activities of wild-type IL-7. In some aspects, the IL-7 protein can be derived from humans, rats, mice, monkeys, cows, or sheep.

In some aspects, the human IL-7 can have an amino acid sequence represented by SEQ ID NO: 1 (Genbank Accession No. P13232); the rat IL-7 can have an amino acid sequence represented by SEQ ID NO: 2 (Genbank Accession No. P56478); the mouse IL-7 can have an amino acid sequence represented by SEQ ID NO: 3 (Genbank Accession No. P10168); the monkey IL-7 may have an amino acid sequence represented by SEQ ID NO: 4 (Genbank Accession No. NP 001279008); the cow IL-7 can have an amino acid sequence represented by SEQ ID NO: 5 (Genbank Accession No. P26895), and the sheep IL-7 can have an amino acid sequence represented by SEQ ID NO: 6 (Genbank Accession No. Q28540).

In some aspects, an IL-7 protein useful for the present disclosure comprises an IL-7 fusion protein. In certain aspects, an IL-7 fusion protein comprises (i) an oligopeptide and (i) an IL-7 or a variant thereof. In some aspects, the oligopeptide is linked to the N-terminal region of the IL-7 or a variant thereof.

In some aspects, an oligopeptide disclosed herein consists of 1 to 10 amino acids. In certain aspects, an oligopeptide consists of at least 1, at least 2, at least 3, at least 4, at least 5, at least 6, at least 7, at least 8, at least 9, or 10 amino acids. In some aspects, one or more amino acids of an oligopeptide are selected from the group consisting of methionine, glycine, and combinations thereof. In certain aspects, an oligopeptide is selected from the group consisting of methionine (M), glycine (G), methionine-methionine (MM), glycine-glycine (GG), methionine-glycine (MG), glycine-methionine (GM), methionine-methionine-methionine (MMM), methionine-methionine-glycine (MMG), methionine-glycine-methionine (MGM), glycine-methionine-methionine (GMM), methionine-glycine-glycine (MGG), glycine-methionine-glycine (GMG), glycine-glycine-methionine (GGM), glycine-glycine-glycine (GGG), methionine-glycine-glycine-methionine (MGGM) (SEQ ID NO: 41), methionine-methionine-glycine-glycine (MMGG) (SEQ ID NO: 42), glycine-glycine-methionine-methionine (GGMM) (SEQ ID NO: 43), methionine-glycine-methionine-glycine (MGMG) (SEQ ID NO: 44), glycine-methionine-methionine-glycine (GMMG) (SEQ ID NO: 45), glycine-glycine-glycine-methionine (GGGM) (SEQ ID NO: 46), methionine-glycine-glycine-glycine (MGGG) (SEQ ID NO: 47), glycine-methionine-glycine-glycine (GMGG) (SEQ ID NO: 48), glycine-glycine-methionine-glycine (GGMG) (SEQ ID NO: 49), glycine-glycine-methionine-methionine-methionine (GGMMM) (SEQ ID NO: 50), glycine-glycine-glycine-methionine-methionine (GGGMM) (SEQ ID NO: 51), glycine-glycine-glycine-glycine-methionine (GGGGM) (SEQ ID NO: 52), methionine-glycine-methionine-methionine-methionine (MGMMM) (SEQ ID NO: 53), methionine-glycine-glycine-methionine-methionine (MGGMM) (SEQ ID NO: 54), methionine-glycine-glycine-glycine-methionine (MGGGM) (SEQ ID NO: 55), methionine-methionine-glycine-methionine-methionine (MMGMM) (SEQ ID NO: 56), methionine-methionine-glycine-glycine-methionine (MMGGM) (SEQ ID NO: 57), methionine-methionine-glycine-glycine-glycine (MMGGG) (SEQ ID NO: 58), methionine-methionine-methionine-glycine-methionine (MMMGM) (SEQ ID NO: 59), methionine-glycine-methionine-glycine-methionine (MGMGM) (SEQ ID NO: 60), glycine-methionine-glycine-methionine-glycine (GMGMG) (SEQ ID NO: 61), glycine-methionine-methionine-methionine-glycine (GMMMG) (SEQ ID NO: 62), glycine-glycine-methionine-glycine-methionine (GGMGM) (SEQ ID NO: 63), glycine-glycine-methionine-methionine-glycine (GGMMG) (SEQ ID NO: 64), glycine-methionine-methionine-glycine-methionine (GMMGM) (SEQ ID NO: 65), methionine-glycine-methionine-methionine-glycine (MGMMG) (SEQ ID NO: 66), glycine-methionine-glycine-glycine-methionine (GMGGM) (SEQ ID NO: 67), methionine-methionine-glycine-methionine-glycine (MMGMG) (SEQ ID NO: 68), glycine-methionine-methionine-glycine-glycine (GMMGG) (SEQ ID NO: 69), glycine-methionine-glycine-glycine-glycine (GMGGG) (SEQ ID NO: 70), glycine-glycine-methionine-glycine-glycine (GGMGG) (SEQ ID NO: 71), glycine-glycine-glycine-glycine-glycine (GGGGG) (SEQ ID NO: 72), or combinations thereof. In some aspects, an oligopeptide is methionine-glycine-methionine (MGM).

In some aspects, an IL-7 fusion protein comprises (i) an IL-7 or a variant thereof, and (ii) a half-life extending moiety. In some aspects, a half-life extending moiety extends the half-life of the IL-7 or variant thereof. In some aspects, a half-life extending moiety is linked to the C-terminal region of an IL-7 or a variant thereof.

In some aspects, an IL-7 fusion protein comprises (i) IL-7 (a first domain), (ii) a second domain that includes an amino acid sequence having 1 to 10 amino acid residues consisting of methionine, glycine, or a combination thereof, e.g., MGM, and (iii) a third domain comprising a half-life extending moiety. In some aspects, the half-life extending moiety can be linked to the N-terminal or the C-terminal of the first domain or the second domain. Additionally, the IL-7 including the first domain and the second domain can be linked to both terminals of the third domain.

Non-limiting examples of half-life extending moieties include: Fc, albumin, an albumin-binding polypeptide, Pro/Ala/Ser (PAS), a C-terminal peptide (CTP) of the subunit of human chorionic gonadotropin, polyethylene glycol (PEG), long unstructured hydrophilic sequences of amino acids (XTEN), hydroxyethyl starch (HES), an albumin-binding small molecule, and combinations thereof.

In some aspects, a half-life extending moiety is Fc. In certain aspects, Fc is from a modified immunoglobulin in which the antibody-dependent cellular cytotoxicity (ADCC) or complement-dependent cytotoxicity (CDC) weakened due to the modification in the binding affinity with the Fc receptor and/or a complement. In some aspects, the modified immunoglobulin can be selected from the group consisting of IgG1, IgG2, IgG3, IgG4, IgA1, IgA2, IgD, IgE, and a combination thereof. In some aspects, an Fc is a hybrid Fc (“hFc” or “hyFc”), comprising a hinge region, a CH2 domain, and a CH3 domain. In certain aspects, a hinge region of a hybrid Fc disclosed herein comprises a human IgD hinge region. In certain aspects, a CH2 domain of a hybrid Fc comprises a part of human IgD CH2 domain and a part of human IgG4 CH2 domain. In certain aspects, a CH3 domain of a hybrid Fc comprises a part of human IgG4 CH3 domain. Accordingly, in some aspects, a hybrid Fc disclosed herein comprises a hinge region, a CH2 domain, and a CH3 domain, wherein the hinge region comprises a human IgD hinge region, wherein the CH2 domain comprises a part of human IgD CH2 domain and a part of human IgG4 CH2 domain, and wherein the CH3 domain comprises a part of human IgG4 CH3 domain.

In some aspects, an Fc disclosed herein can be an Fc variant. As used herein, the term “Fc variant” refers to an Fc which was prepared by substituting a part of the amino acids among the Fc region or by combining the Fc regions of different kinds. The Fc region variant can prevent from being cut off at the hinge region. Specifically, in some aspects, a Fc variant comprises modifications at the 144^{th} amino acid and/or 145^{th} amino acid of SEQ ID NO: 9. In certain aspects, the 144^{th} amino acid (K) and/or the 145^{th} amino acid (K) is substituted with G or S.

In some aspects, an Fc or an Fc variant disclosed herein can be represented by the following formula: N′—(Z1)p-Y-Z2-Z3-Z4-C, wherein:

N′ comprises the N-terminus;

Z1 comprises an amino acid sequence having 5 to 9 consecutive amino acid residues from the amino acid residue at position 98 toward the N-terminal, among the amino acid residues at positions from 90 to 98 of SEQ ID NO: 7;

Y comprises an amino acid sequence having 5 to 64 consecutive amino acid residues from the amino acid residue at position 162 toward the N-terminal, among the amino acid residues at positions from 99 to 162 of SEQ ID NO: 7;

Z2 comprises an amino acid sequence having 4 to 37 consecutive amino acid residues from the amino acid residue at position 163 toward the C-terminal, among the amino acid residues at positions from 163 to 199 of SEQ ID NO: 7;

Z3 comprises an amino acid sequence having 71 to 106 consecutive amino acid residues from the amino acid residue at position 220 toward the N-terminal, among the amino acid residues at positions from 115 to 220 of SEQ ID NO: 8; and

Z4 comprises an amino acid sequence having 80 to 107 consecutive amino acid residues from the amino acid residue at position 221 toward the C-terminal, among the amino acid residues at positions from 221 to 327 of SEQ ID NO: 8.

In some aspects, a Fc region disclosed herein can include the amino acid sequence of SEQ ID NO: 9 (hyFc), SEQ ID NO: 10 (hyFcM1), SEQ ID NO: 11 (hyFcM2), SEQ ID NO: 12 (hyFcM3), or SEQ ID NO: 13 (hyFcM4). In some aspects, the Fc region can include the amino acid sequence of SEQ ID NO: 14 (a non-lytic mouse Fc).

Other non-limiting examples of Fc regions that can be used with the present disclosure are described in U.S. Pat. No. 7,867,491, which is herein incorporated by reference in its entirety.

In some aspects, an IL-7 fusion protein disclosed herein comprises both an oligopeptide and a half-life extending moiety.

In some aspects, an IL-7 protein can be fused to albumin, a variant, or a fragment thereof. Examples of the IL-7-albumin fusion protein can be found at International Application Publication No. WO 2011/124718 A1. In some aspects, an 1L-7 protein is fused to a pre-pro-B cell Growth Stimulating Factor (PPBSF), optionally by a flexible linker. See US 2002/0058791A1. In some aspects, an IL-7 protein useful for the disclosure is an IL-7 conformer that has a particular three dimensional structure. See US 2005/0249701 A1. In some aspects, an IL-7 protein can be fused to an Ig chain, wherein amino acid residues 70 and 91 in the IL-7 protein are glycosylated the amino acid residue 116 in the IL-7 protein is non-glycosylated. See U.S. Pat. No. 7,323,549 B2. In some aspects, an IL-7 protein that does not contain potential T-cell epitopes (thereby to reduce anti-IL-7 T-cell responses) can also be used for the present disclosure. See US 2006/0141581 A1. In some aspects, an IL-7 protein that has one or more amino acid residue mutations in carboxy-terminal helix D region can be used for the present disclosure. The IL-7 mutant can act as IL-7R partial agonist despite lower binding affinity for the receptor. See US 2005/0054054A1. Any IL-7 proteins described in the above listed patents or publications are incorporated herein by reference in their entireties.

In addition, non-limiting examples of additional IL-7 proteins useful for the present disclosure are described in U.S. Pat. Nos. 7,708,985, 8,034,327, 8,153,114, 7,589,179, 7,323,549, 7,960,514, 8,338,575, 7,118,754, 7,488,482, 7,670,607, 6,730,512, WO0017362, GB2434578A, WO 2010/020766 A2, WO91/01143, Beq et al., Blood, vol. 114 (4), 816, 23 Jul. 2009, Kang et al., J. Virol. Doi:10.1128/JVI.02768-15, Martin et al., Blood, vol. 121 (22), 4484, May 30, 2013, McBride et al., Acta Oncologica, 34:3, 447-451, Jul. 8, 2009, and Xu el al., Cancer Science, 109: 279-288, 2018, which are incorporated herein by reference in their entireties.

In some aspects, an oligopeptide disclosed herein is directly linked to the N-terminal region of IL-7 or a variant thereof. In some aspects, an oligopeptide is linked to the N-terminal region via a linker. In some aspects, a half-life extending moiety disclosed herein is directly linked to the C-terminal region of IL-7 or a variant thereof. In certain aspects, a half-life extending moiety is linked to the C-terminal region via a linker. In some aspects, a linker is a peptide linker. In certain aspects, a peptide linker comprises a peptide of 10 to 20 amino acid residues consisting of Gly and Ser residues. In some aspects, a linker is an albumin linker. In some aspects, a linker is a chemical bond. In certain aspects, a chemical bond comprises a disulfide bond, a diamine bond, a sulfide-amine bond, a carboxy-amine bond, an ester bond, a covalent bond, or combinations thereof. When the linker is a peptide linker, in some aspects, the connection can occur in any linking region. They may be coupled using a crosslinking agent known in the art. In some aspects, examples of the crosslinking agent can include N-hydroxy succinimide esters such as 1,1-bis(diazoacetyl)-2-phenylethane, glutaraldehyde, and 4-azidosalicylic acid; imido esters including disuccinimidyl esters such as 3,3′-dithiobis (succinimidyl propionate), and bifunctional maleimides such as bis-Nmaleimido-1,8-octane, but is not limited thereto.

In some aspects, an IL-7 (or variant thereof) portion of IL-7 fusion protein disclosed herein comprises an amino sequence that is at least 70%, at least 75%, at least 80%, at least 85%, at least 90%, at least 91%, at least 92%, at least 93%, at least 94%, at least 95%, at least 96%, at least 97%, at least 98%, at least 98%, or at least 99% identical to an amino acid sequence set forth in SEQ ID NOs: 15-20. In certain aspects, an IL-7 (or variant thereof) portion of IL-7 fusion protein disclosed herein comprises the amino acid sequence set forth in SEQ ID NOs: 15-20.

In some aspects, an IL-7 fusion protein comprises: a first domain including a polypeptide having the activity of IL-7 or a similar activity thereof; a second domain comprising an amino acid sequence having 1 to 10 amino acid residues consisting of methionine, glycine, or a combination thereof; and a third domain, which is an Fc region of modified immunoglobulin, coupled to the C-terminal of the first domain.

In some aspects, an IL-7 fusion protein that can be used with the present methods comprises an amino sequence that is at least 70%, at least 75%, at least 80%, at least 85%, at least 90%, at least 91%, at least 92%, at least 93%, at least 94%, at least 95%, at least 96%, at least 97%, at least 98%, at least 98%, or at least 99% identical to an amino acid sequence set forth in SEQ ID NOs: 21-25. In certain aspects, an IL-7 fusion protein of the present disclosure comprises the amino acid sequence set forth in SEQ ID NOs: 21-25. In certain aspects, an IL-7 fusion protein disclosed herein comprises the amino acid sequence set forth in SEQ ID NOs: 26 and 27.

In some aspects, an IL-7 protein useful for the present disclosure can increase absolute lymphocyte counts in a subject when administered to the subject. In certain aspects, the subject suffers from a disease or disorder described herein (e.g., cancer). In some aspects, the subject is a healthy individual (e.g., does not suffer from a disease or disorder described herein, e.g., cancer). In certain aspects, the absolute lymphocyte count is increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% or more, compared to a reference (e.g., corresponding level in a subject that did not receive the IL-7 protein).

### IV. CAR-Bearing Immune Cells

A. Mono CAR-T Cells

As described herein, in some aspects, CAR-bearing immune cells that can be administered in combination with an IL-7 protein disclosed herein, e.g., to treat a solid tumor, comprises CAR-T cells. As used herein, the term “CAR-T cells” refers to T cells that express a chimeric antigen receptor (CAR). The term “chimeric antigen receptor” refers to a recombinant fusion protein that has an antigen-specific extracellular domain coupled to an intracellular domain that directs the cell to perform a specialized function upon binding of an antigen to the extracellular domain. The terms “artificial T cell receptor,” “chimeric T-cell receptor,” and “chimeric immunoreceptor” can each be used interchangeably herein with the term “chimeric antigen receptor.” Chimeric antigen receptors are distinguished from other antigen binding agents by their ability to both bind MEC-independent antigen and transduce activation signals via their intracellular domain. The extracellular and intracellular portions of a CAR are discussed in more detail below.

The antigen-specific extracellular domain (i.e., antigen-binding domain) of a chimeric antigen receptor recognizes and specifically binds an antigen, typically a surface-expressed antigen of a malignancy. In some aspects, the antigen-binding domain of a CAR disclosed herein binds to a solid tumor antigen (i.e., an antigen expressed on solid tumors). Non-limiting examples of solid tumor antigens include mesothelin, MR1, guanylate cyclase C (GC-C), epidermal growth factor receptor (EGFR or erbB-1), human epidermal growth factor receptor 2 (HER2 or erbB2), erbB-3, erbB-4, MUC-1, melanoma-associated chondroitin sulfate proteoglycan (MCSP), folate receptor 1 (FOLR1), CD4, CD19, CD20, CD22, CD30, CD33, CD38, CD44, CD44v6, CD44v7/8, CD70, CD123, CD138, CD171, CEA, CSPG4, CXCR5, c-Met, HERV-envelope protein, eriostin, Bigh3, SPARC, BCR, CD79, CD37, EGFRvIII, EGP2, EGP40, IGFr, L1 CAM, AXL, Tissue Factor (TF), CD74, EpCAM, EphA2, MRP3cadherin 19 (CDH19), epidermal growth factor 2 (HER2), 5T4, 8H9, αᵥβ₆ integrin, BCMA, B7-H3, B7-H6, CAIX, CA9, FAP, FBP, fetal AchR, FRcc, GD2, GD3, Glypican-1 (GPC1), Glypican-2 (GPC2), Glypican-3 (GPC3), HLA-A1+MAGE1, HLA-A1+NY-ESO-1, IL-13Rcc2, Lewis-Y, KDR, MCSP, Muc1, Muc16, NCAM, NKG2D ligands, NY-ESO-1, PRAME, PSC1, PSCA, PSMA, ROR1, ROR2, SP17, surviving, TAG72, TEMs, carcinoembryonic antigen, HMW-MAA, VEGF, CLDN18.2, or combinations thereof.

In some aspects, the antigen-specific extracellular domain of a CAR disclosed herein specifically binds an antigen (e.g., solid tumor antigen) with an affinity constant or affinity of interaction (K_{D}) between about 0.1 pM to about 10 for example, about 0.1 pM to about 1 μM or about 0.1 pM to about 100 nM. Methods for determining the affinity of interaction are known in the art. An antigen-specific extracellular domain suitable for use in a CAR of the present disclosure can be any antigen-binding polypeptide, a wide variety of which are known in the art. In some aspects, the antigen-binding domain is a single chain Fv (scFv). Other antibody-based recognition domains (cAb VHH (camelid antibody variable domains) and humanized versions thereof, lgNAR VH (shark antibody variable domains) and humanized versions thereof, sdAb VH (single domain antibody variable domains) and “camelized” antibody variable domains are suitable for use. In some aspects, T cell receptor (TCR) based recognition domains, such as single chain TCR (scTv, single chain two-domain TCR containing V.alpha.V.beta.) are also suitable for use.

As described herein, a chimeric antigen receptor disclosed herein can also include an intracellular domain that provides an intracellular signal to the cell (expressing the CAR) upon antigen binding to the antigen-specific extracellular domain. In some aspects, the intracellular signaling domain of a CAR is responsible for activation of at least one of the effector functions of the T cell in which the chimeric receptor is expressed.

The term “intracellular domain” refers to the portion of a CAR that transduces the effector function signal upon binding of an antigen to the extracellular domain and directs the T cell to perform a specialized function. Non-limiting examples of suitable intracellular domains include the zeta chain of the T-cell receptor or any of its homologs (e.g., eta, delta, gamma, or epsilon), MB1 chain, 829, FcγRIII, FcγRI, and combinations of signaling molecules, such as CD3.zeta. and CD28, CD27, 4-1BB, DAP-10, OX40, and combinations thereof, as well as other similar molecules and fragments. In some aspects, a CAR disclosed herein comprises the entire intracellular domain of a protein disclosed herein. In some aspects, the intracellular domain is truncated. Truncated portion of an intracellular domain can be used in place of the intact chain as long as it still transduces the effector function signal. The term intracellular domain is thus meant to include any truncated portion of the intracellular domain sufficient to transduce the effector function signal.

In some aspects, the antigen-specific extracellular domain (i.e., antigen-binding domain) is linked to the intracellular domain of the chimeric antigen receptor by a transmembrane domain. A transmembrane domain traverses the cell membrane, anchors the CAR to the T cell surface, and connects the extracellular domain to the intracellular signaling domain, thus impacting expression of the CAR on the T cell surface. In some aspects, the antigen-specific extracellular domain is connected to the transmembrane domain of a CAR by a peptide hinge or spacer. In certain aspects, inclusion of a spacer domain between the antigen-specific extracellular domain and the transmembrane domain, and between multiple scFvs in the case of tandem CAR, can affect flexibility of the antigen-binding domain(s) and thereby CAR function.

In some aspects, CARs disclosed herein can further comprise one or more costimulatory domains. In some aspects, the costimulatory domain is derived from intracellular signaling domains of costimulatory proteins that can enhance cytokine production, proliferation, cytotoxicity, and/or persistence in vivo. In some aspects, the transmembrane domain is fused to the costimulatory domain, optionally a costimulatory domain is fused to a second costimulatory domain, and the costimulatory domain is fused to a signaling domain, not limited to CD3ζ. Suitable transmembrane domains, costimulatory domains, and spacers that can be used in CARs disclosed herein are known in the art.

B. Genome Edited CAR-T Cells

In some aspects, CAR-bearing immune cells that can be used with the present disclosure are genome-edited CAR-T cells. In some aspects, such cells have been modified so that the cells are deficient in one or more antigens to which the CARs specifically binds. Accordingly, in some aspects, the CAR-bearing immune cells disclosed herein are fratricide-resistant. In some aspects, the one or more antigens of an immune cell (e.g., T cells) are modified such the chimeric antigen receptor no longer specifically binds the one or more modified antigens. For example, the epitope of the one or more antigens recognized by the chimeric antigen receptor can be modified by one or more amino acid changes (e.g., substitutions or deletions) or the epitope can be deleted from the antigen. In some aspects, expression of the one or more antigens is reduced in the immune cells (e.g., T cells) by at least about 5%, at least about 10%, at least about 20%, at least about 30%, at least about 40%, at least about 50%, at least about 60%, at least about 70%, at least about 80%, at least about 90% or more. Methods for decreasing the expression of a protein are known in the art and include, but are not limited to, modifying or replacing the promoter operably linked to the nucleic acid sequence encoding the protein. In some aspects, the CAR-bearing immune cells (e.g., CAR-T cells) are modified such that the one or more antigens are not expressed, e.g., by deletion or disruption of the gene encoding the one or more antigens. Methods for genetically modifying an immune cell (e.g., CAR-T cell) to be deficient in one or more antigens are well known in art (e.g., CRISPR/cas9 gene editing).

In some aspects, CAR-T cells encompassed by the present disclosure can further be deficient in endogenous T cell receptor (TCR) signaling as a result of deleting a part of the T Cell Receptor (TCR)-CD3 complex. In some aspects, decreasing or eliminating endogenous TCR signaling in CAR-T cells can prevent or reduce graft versus host disease (GvHD) when allogenic T cells are used to produce the CAR-T cells. Methods for eliminating or suppressing endogenous TCR signaling are known in the art and include, but are not limited to, deleting a part of the TCR-CD3 receptor complex, e.g., the TCR receptor alpha chain (TRAC), the TCR receptor beta chain (TRBC), CD3.epsilon, CD3.gamma, CD3.delta, and/or CD3.gamma. Deleting a part of the TCR receptor complex can block TCR mediated signaling and can thus permit the safe use of allogeneic T cells as the source of CAR-T cells without inducing life-threatening GvHD.

In some aspects, CAR-T cells that are useful for the present disclosure can further comprise one or more suicide genes. As used herein, “suicide gene” refers to a nucleic acid sequence introduced to a CAR-T cell by standard methods known in the art that, when activated, results in the death of the CAR-T cell. Suicide genes can facilitate effective tracking and elimination of the CAR-T cells in vivo if required. Facilitated killing by activating the suicide gene can occur by methods known in the art. Suitable suicide gene therapy systems known in the art include, but are not limited to, various the herpes simplex virus thymidine kinase (HSVtk)/ganciclovir (GCV) suicide gene therapy systems or inducible caspase 9 protein. In certain aspects, a suicide gene is a CD34/thymidine kinase chimeric suicide gene.

C. Dual CAR-T Cells

In some aspects, CAR-bearing immune cells are dual CAR-T cells. As used herein, the term “dual CAR-T cells” are CAR-T cells that express two distinct chimeric antigen receptor polypeptides with affinity to different target antigens expressed within the same effector cell, wherein each CAR functions independently. The CAR can be expressed from a single polynucleotide sequence or multiple polynucleotide sequences. Non-limiting examples of dual CAR-T cells are described below.

A genome-edited, dual CAR-T cell, i.e., CD2*CD3e-dCARTΔCD2ΔCD3Δ, can be generated by cloning a commercially synthesized anti-CD2 single chain variable fragment into a lentiviral vector containing a 3rd generation CAR backbone with CD28 and 4-1BB internal signaling domains and cloning a commercially synthesized anti-CD3e single chain variable into the same lentiviral vector containing an additional 3rd generation CAR backbone with CD28 and 4-1BB internal signaling domains resulting in a plasmid from which the two CAR constructs are expressed from the same vector.

In some aspects, the disclosure provides an engineered T cell comprising a dual Chimeric Antigen Receptor (dCAR), i.e., two CARs expressed from a single lentivirus construct, that specifically binds CD5 and TCR receptor alpha chain (TRAC), wherein the T cell is deficient in CD5 and TRAC (e.g., CD5*TRAC-dCARTΔCD5ΔTRAC cell). In non-limiting examples the deficiency in CD5 and the TCR receptor alpha chain (TRAC) resulted from (a) modification of CD5 and the TCR receptor alpha chain (TRAC) expressed by the T cell such that the chimeric antigen receptor no longer specifically binds the modified CD5 and the TCR receptor alpha chain (TRAC), (b) modification of the T cell such that expression of the CD5 and the TCR receptor alpha chain (TRAC) is reduced in the T cell by at least about 10%, at least about 20%, at least about 30%, at least about 40%, at least about 50%, at least about 60%, at least about 70%, at least about 80%, at least about 90% or more, or (c) modification of the T cell such that CD5 and the TCR receptor alpha chain (TRAC) is not expressed (e.g., by deletion or disruption of the gene encoding CD5 and/or the TCR receptor alpha chain (TRAC). In some aspects, the T cell comprises a suicide gene. In non-limiting examples the suicide gene expressed in the CD5*TRAC-CARTΔCD5ΔTRAC cells encodes a modified Human-Herpes Simplex Virus-1-thymidine kinase (TK) gene fused in-frame to the extracellular and transmembrane domains of the human CD34 cDNA.

In some aspects, the disclosure provides an engineered T cell compromising a dCAR that specifically binds CD7 and TCR receptor alpha chain (TRAC), wherein the T cell is deficient in CD7 and TRAC (e.g., CD7*TRAC-dCARTΔCD7ΔTRAC cell). In non-limiting examples the deficiency in CD7 and the TCR receptor alpha chain (TRAC) resulted from (a) modification of CD5 and the TCR receptor alpha chain (TRAC) expressed by the T cell such that the chimeric antigen receptor no longer specifically binds the modified CD7 and the TCR receptor alpha chain (TRAC), (b) modification of the T cell such that expression of the CD7 and the TCR receptor alpha chain (TRAC) is reduced in the T cell by at least 50%, at least 60%, at least 70%, at least 80%, at least 90% or more, or (c) modification of the T cell such that CD7 and the TCR receptor alpha chain (TRAC) is not expressed (e.g., by deletion or disruption of the gene encoding CD7 and/or the TCR receptor alpha chain (TRAC). In some aspects, the T cell comprises a suicide gene. In non-limiting examples the suicide gene expressed in the CD7*TRAC-dCARTΔCD7ΔTRAC cells encodes a modified Human-Herpes Simplex Virus-1-thymidine kinase (TK) gene fused in-frame to the extracellular and transmembrane domains of the human CD34 eDNA.

In some aspects, the disclosure provides an engineered T cell compromising a dCAR that specifically binds CD2 and TCR receptor alpha chain (TRAC), wherein the T cell is deficient in CD2 and TRAC (e.g., CD2*TRAC-dCARTΔCD2ΔTRAC cell). In non-limiting examples the deficiency in CD2 and the TCR receptor alpha chain (TRAC) resulted from (a) modification of CD2 and the TCR receptor alpha chain (TRAC) expressed by the T cell such that the chimeric antigen receptor no longer specifically binds the modified CD2 and the TCR receptor alpha chain (TRAC), (b) modification of the T cell such that expression of the CD7 and the TCR receptor alpha chain (TRAC) is reduced in the T cell by at least 50%, at least 60%, at least 70%, at least 80%, at least 90% or more, or (c) modification of the T cell such that CD2 and the TCR receptor alpha chain (TRAC) is not expressed (e.g., by deletion or disruption of the gene encoding CD2 and/or the TCR receptor alpha chain (TRAC). In some aspects, the T cell comprises a suicide gene. In non-limiting examples the suicide gene expressed in the CD2*TRAC-dCARTΔCD2ΔTRAC cells encodes a modified Human-Herpes Simplex Virus-1-thymidine kinase (TK) gene fused in-frame to the extracellular and transmembrane domains of the human CD34 cDNA.

D. Tandem CAR-T Cells

In some aspects, CAR-bearing immune cells are tandem CAR-T cells. As used herein, the term “tandem CAR-T cells” refer to CAR-T cells with a single chimeric antigen polypeptide containing two distinct antigen recognition domains with affinity to different targets wherein the antigen recognition domain are linked through a peptide linker and share common costimulatory domain (s), wherein binding of either antigen recognition domain will signal though a common costimulatory domains(s) and signaling domain. Non-limiting examples of tandem CAR-T cells are described below.

In some aspects, the disclosure provides an engineered T cell comprising a tandem Chimeric Antigen Receptor (tCAR), i.e., two scFv sharing a single intracellular domain, that specifically binds CD5 and TCR receptor alpha chain (TRAC), wherein the T cell is deficient in CD5 and TRAC (e.g., CD5*TRAC-tCARTΔCD5ΔTRAC cell). In non-limiting examples the deficiency in CD5 and the TCR receptor alpha chain (TRAC) resulted from (a) modification of CD5 and the TCR receptor alpha chain (TRAC) expressed by the T cell such that the chimeric antigen receptor no longer specifically binds the modified CD5 and the TCR receptor alpha chain (TRAC), (b) modification of the T cell such that expression of the CD5 and the TCR receptor alpha chain (TRAC) is reduced in the T cell by at least about 10%, at least about 20%, at least about 30%, at least about 40%, at least about 50%, at least about 60%, at least about 70%, at least about 80%, at least about 90% or more, or (c) modification of the T cell such that CD5 and the TCR receptor alpha chain (TRAC) is not expressed (e.g., by deletion or disruption of the gene encoding CD5 and/or the TCR receptor alpha chain (TRAC). In some aspects, the T cell comprises a suicide gene. In non-limiting examples the suicide gene expressed in the CD5*TRAC-tCARTΔCD5ΔTRAC cells encodes a modified Human-Herpes Simplex Virus-1-thymidine kinase (TK) gene fused in-frame to the extracellular and transmembrane domains of the human CD34 cDNA.

In some aspects, the disclosure provides an engineered T cell compromising a tCAR that specifically binds CD7 and TCR receptor alpha chain (TRAC), wherein the T cell is deficient in CD7 and TRAC (e.g., CD7*TRAC-tCARTΔCD7ΔTRAC cell). In non-limiting examples the deficiency in CD7 and the TCR receptor alpha chain (TRAC) resulted from (a) modification of CD5 and the TCR receptor alpha chain (TRAC) expressed by the T cell such that the chimeric antigen receptor no longer specifically binds the modified CD7 and the TCR receptor alpha chain (TRAC), (b) modification of the T cell such that expression of the CD7 and the TCR receptor alpha chain (TRAC) is reduced in the T cell by at least about 10%, at least about 20%, at least about 30%, at least about 40%, at least about 50%, at least about 60%, at least about 70%, at least about 80%, at least about 90% or more, or (c) modification of the T cell such that CD7 and the TCR receptor alpha chain (TRAC) is not expressed (e.g., by deletion or disruption of the gene encoding CD7 and/or the TCR receptor alpha chain (TRAC). In some aspects, the T cell comprises a suicide gene. In non-limiting examples the suicide gene expressed in the CD7*TRAC-tCARTΔCD7ΔTRAC cells encodes a modified Human-Herpes Simplex Virus-1-thymidine kinase (TK) gene fused in-frame to the extracellular and transmembrane domains of the human CD34 cDNA.

In some aspects, the disclosure provides an engineered T cell compromising a tCAR that specifically binds CD2 and TCR receptor alpha chain (TRAC), wherein the T cell is deficient in CD2 and TRAC (e.g., CD2*TRAC-tCARTΔCD2ΔTRAC cell). In non-limiting examples the deficiency in CD2 and the TCR receptor alpha chain (TRAC) resulted from (a) modification of CD2 and the TCR receptor alpha chain (TRAC) expressed by the T cell such that the chimeric antigen receptor no longer specifically binds the modified CD2 and the TCR receptor alpha chain (TRAC), (b) modification of the T cell such that expression of the CD7 and the TCR receptor alpha chain (TRAC) is reduced in the T cell by at least about 10%, at least about 20%, at least about 30%, at least about 40%, at least about 50%, at least about 60%, at least about 70%, at least about 80%, at least about 90% or more, or (c) modification of the T cell such that CD2 and the TCR receptor alpha chain (TRAC) is not expressed (e.g., by deletion or disruption of the gene encoding CD2 and/or the TCR receptor alpha chain (TRAC). In some aspects, the T cell comprises a suicide gene. In non-limiting examples the suicide gene expressed in the CD2*TRAC-tCARTΔCD2ΔTRAC cells encodes a modified Human-Herpes Simplex Virus-1-thymidine kinase (TK) gene fused in-frame to the extracellular and transmembrane domains of the human CD34 cDNA.

Non-limiting examples of CAR-T cells (e.g., mono CAR-T cells, genome-edited CAR-T cells, dual CAR-T cells, or tandem CAR-T cells) that can be constructed and used with the present disclosure are provided in Tables 1 and 2.

TABLE 1
Exemplary mono CAR-T cells and mono iNKT-CARs

| | Antigen Target of | |
|---|---|---|
| | a mono CAR-T or | Antigen Deletion/ |
| Example | a mono iNKT-CAR | Suppression |
| 1 | CD2 | — |
| 2 | CD3ε | — |
| 3 | CD4 | — |
| 4 | CD5 | — |
| 5 | CD7 | — |
| 6 | TRAC | — |
| 7 | TCRβ | — |
| 8 | CD2 | CD2 |
| 9 | CD3ε | CD3ε |
| 10 | CD4 | CD4 |
| 11 | CD5 | CD5 |
| 12 | CD7 | CD7 |
| 13 | TRAC | TRAC |
| 14 | TCRβ | TCRβ |

TABLE 2
Exemplary dual and tandem CAR-T cells and iNKT-CARs

| | Antigen Target of | Antigen Deletion/ | Tandem |
|---|---|---|---|
| Example | CAR-T or INKT-CAR | Suppression | or Dual |
| 15 | CD2 × CD3ε | — | Tandem |
| 16 | CD2 × CD3ε | CD2 | Tandem |
| 17 | CD2 × CD3ε | CD3ε | Tandem |
| 18 | CD2 × CD3ε | CD2 and CD3ε | Tandem |
| 19 | CD2 × CD4 | — | Tandem |
| 20 | CD2 × CD4 | CD2 | Tandem |
| 21 | CD2 × CD4 | CD4 | Tandem |
| 22 | CD2 × CD4 | CD2 and CD4 | Tandem |
| 23 | CD2 × CD5 | — | Tandem |
| 24 | CD2 × CD5 | CD2 | Tandem |
| 25 | CD2 × CD5 | CD5 | Tandem |
| 26 | CD2 × CD5 | CD2 and CD5 | Tandem |
| 27 | CD2 × CD7 | — | Tandem |
| 28 | CD2 × CD7 | CD2 | Tandem |
| 29 | CD2 × CD7 | CD7 | Tandem |
| 30 | CD2 × CD7 | CD2 and CD7 | Tandem |
| 31 | CD3ε × CD4 | — | Tandem |
| 32 | CD3ε × CD4 | CD3ε | Tandem |
| 33 | CD3ε × CD4 | CD4 | Tandem |
| 34 | CD3ε × CD4 | CD3ε and CD4 | Tandem |
| 35 | CD3ε × CD5 | — | Tandem |
| 36 | CD3ε × CD5 | CD3ε | Tandem |
| 37 | CD3ε × CD5 | CD5 | Tandem |
| 38 | CD3ε × CD5 | CD3ε and CD5 | Tandem |
| 39 | CD3ε × CD7 | — | Tandem |
| 40 | CD3ε × CD7 | CD3ε | Tandem |
| 41 | CD3ε × CD7 | CD7 | Tandem |
| 42 | CD3ε × CD7 | CD3ε and CD7 | Tandem |
| 43 | CD4 × CD5 | — | Tandem |
| 44 | CD4 × CD5 | CD4 | Tandem |
| 45 | CD4 × CD5 | CD5 | Tandem |
| 46 | CD4 × CD5 | CD4 and CD5 | Tandem |
| 47 | CD4 × CD7 | — | Tandem |
| 48 | CD4 × CD7 | CD4 | Tandem |
| 49 | CD4 × CD7 | CD7 | Tandem |
| 50 | CD4 × CD7 | CD5 and CD7 | Tandem |
| 51 | CD5 × CD7 | — | Tandem |
| 52 | CD5 × CD7 | CD5 | Tandem |
| 53 | CD5 × CD7 | CD7 | Tandem |
| 54 | CD5 × CD7 | CD5 and CD7 | Tandem |
| 55 | TRAC × CD2 | — | Tandem |
| 56 | TRAC × CD2 | TRAC | Tandem |
| 57 | TRAC × CD2 | CD2 | Tandem |
| 58 | TRAC × CD2 | TRAC and CD2 | Tandem |
| 59 | TRAC × CD3ε | — | Tandem |
| 60 | TRAC × CD3ε | TRAC | Tandem |
| 61 | TRAC × CD3ε | CD3ε | Tandem |
| 62 | TRAC × CD3ε | TRAC and CD3ε | Tandem |
| 63 | TRAC × CD4 | — | Tandem |
| 64 | TRAC × CD4 | TRAC | Tandem |
| 65 | TRAC × CD4 | CD4 | Tandem |
| 66 | TRAC × CD4 | TRAC and CD4 | Tandem |
| 67 | TRAC × CD5 | — | Tandem |
| 68 | TRAC × CD5 | TRAC | Tandem |
| 69 | TRAC × CD5 | CD5 | Tandem |
| 70 | TRAC × CD5 | TRAC and CD5 | Tandem |
| 71 | TRAC × CD7 | — | Tandem |
| 72 | TRAC × CD7 | TRAC | Tandem |
| 73 | TRAC × CD7 | CD7 | Tandem |
| 74 | TRAC × CD7 | TRAC and CD7 | Tandem |
| 75 | TCRβ × CD2 | — | Tandem |
| 76 | TCRβ × CD2 | TCRβ | Tandem |
| 77 | TCRβ × CD2 | CD2 | Tandem |
| 78 | TCRβ × CD2 | TCRβ and CD2 | Tandem |
| 79 | TCRβ × CD3ε | — | Tandem |
| 80 | TCRβ × CD3ε | TCRβ | Tandem |
| 81 | TCRβ × CD3ε | CD3ε | Tandem |
| 82 | TCRβ × CD3ε | TCRβ and CD3ε | Tandem |
| 83 | TCRβ × CD4 | — | Tandem |
| 84 | TCRβ × CD4 | TCRβ | Tandem |
| 85 | TCRβ × CD4 | CD4 | Tandem |
| 86 | TCRβ × CD4 | TCRβ and CD4 | Tandem |
| 87 | TCRβ × CD5 | — | Tandem |
| 88 | TCRβ × CD5 | TCRβ | Tandem |
| 89 | TCRβ × CD5 | CD5 | Tandem |
| 90 | TCRβ × CD5 | TCRβ and CD5 | Tandem |
| 91 | TCRβ × CD7 | — | Tandem |
| 92 | TCRβ × CD7 | TCRβ | Tandem |
| 93 | TCRβ × CD7 | CD7 | Tandem |
| 94 | TCRβ × CD7 | TCRβ and CD7 | Tandem |
| 95 | CD2 × CD3ε | — | Dual |
| 96 | CD2 × CD3ε | CD2 | Dual |
| 97 | CD2 × CD3ε | CD3ε | Dual |
| 98 | CD2 × CD3ε | CD2 and CD3ε | Dual |
| 99 | CD2 × CD4 | — | Dual |
| 100 | CD2 × CD4 | CD2 | Dual |
| 101 | CD2 × CD4 | CD4 | Dual |
| 102 | CD2 × CD4 | CD2 and CD4 | Dual |
| 103 | CD2 × CD5 | — | Dual |
| 104 | CD2 × CD5 | CD2 | Dual |
| 105 | CD2 × CD5 | CD5 | Dual |
| 106 | CD2 × CD5 | CD2 and CD5 | Dual |
| 107 | CD2 × CD7 | — | Dual |
| 108 | CD2 × CD7 | CD2 | Dual |
| 109 | CD2 × CD7 | CD7 | Dual |
| 110 | CD2 × CD7 | CD2 and CD7 | Dual |
| 111 | CD3ε × CD4 | — | Dual |
| 112 | CD3ε × CD4 | CD3ε | Dual |
| 113 | CD3ε × CD4 | CD4 | Dual |
| 114 | CD3ε × CD4 | CD3ε and CD4 | Dual |
| 115 | CD3ε × CD5 | — | Dual |
| 116 | CD3ε × CD5 | CD3ε | Dual |
| 117 | CD3ε × CD5 | CD5 | Dual |
| 118 | CD3ε × CD5 | CD3ε and CD5 | Dual |
| 119 | CD3ε × CD7 | — | Dual |
| 120 | CD3ε × CD7 | CD3ε | Dual |
| 121 | CD3ε × CD7 | CD7 | Dual |
| 122 | CD3ε × CD7 | CD3ε and CD7 | Dual |
| 123 | CD4 × CD5 | — | Dual |
| 124 | CD4 × CD5 | CD4 | Dual |
| 125 | CD4 × CD5 | CD5 | Dual |
| 126 | CD4 × CD5 | CD4 and CD5 | Dual |
| 127 | CD4 × CD7 | — | Dual |
| 128 | CD4 × CD7 | CD4 | Dual |
| 129 | CD4 × CD7 | CD7 | Dual |
| 130 | CD4 × CD7 | CD5 and CD7 | Dual |
| 131 | CD5 × CD7 | — | Dual |
| 132 | CD5 × CD7 | CD5 | Dual |
| 133 | CD5 × CD7 | CD7 | Dual |
| 134 | CD5 × CD7 | CD5 and CD7 | Dual |
| 135 | TRAC × CD2 | — | Dual |
| 136 | TRAC × CD2 | TRAC | Dual |
| 137 | TRAC × CD2 | CD2 | Dual |
| 138 | TRAC × CD2 | TRAC and CD2 | Dual |
| 139 | TRAC × CD3ε | — | Dual |
| 140 | TRAC × CD3ε | TRAC | Dual |
| 141 | TRAC × CD3ε | CD3ε | Dual |
| 142 | TRAC × CD3ε | TRAC and CD3ε | Dual |
| 143 | TRAC × CD4 | — | Dual |
| 144 | TRAC × CD4 | TRAC | Dual |
| 145 | TRAC × CD4 | CD4 | Dual |
| 146 | TRAC × CD4 | TRAC and CD4 | Dual |
| 147 | TRAC × CD5 | — | Dual |
| 148 | TRAC × CD5 | TRAC | Dual |
| 149 | TRAC × CD5 | CD5 | Dual |
| 150 | TRAC × CD5 | TRAC and CD5 | Dual |
| 151 | TRAC × CD7 | — | Dual |
| 152 | TRAC × CD7 | TRAC | Dual |
| 153 | TRAC × CD7 | CD7 | Dual |
| 154 | TRAC × CD7 | TRAC and CD7 | Dual |
| 155 | TCRβ × CD2 | — | Dual |
| 156 | TCRβ × CD2 | TCRβ | Dual |
| 157 | TCRβ × CD2 | CD2 | Dual |
| 158 | TCRβ × CD2 | TCRβ and CD2 | Dual |
| 159 | TCRβ × CD3ε | — | Dual |
| 160 | TCRβ × CD3ε | TCRβ | Dual |
| 161 | TCRβ × CD3ε | CD3ε | Dual |
| 162 | TCRβ × CD3ε | TCRβ and CD3ε | Dual |
| 163 | TCRβ × CD4 | — | Dual |
| 164 | TCRβ × CD4 | TCRβ | Dual |
| 165 | TCRβ × CD4 | CD4 | Dual |
| 166 | TCRβ × CD4 | TCRβ and CD4 | Dual |
| 167 | TCRβ × CD5 | — | Dual |
| 168 | TCRβ × CD5 | TCRβ | Dual |
| 169 | TCRβ × CD5 | CD5 | Dual |
| 170 | TCRβ × CD5 | TCRβ and CD5 | Dual |
| 171 | TCRβ × CD7 | — | Dual |
| 172 | TCRβ × CD7 | TCRβ | Dual |
| 173 | TCRβ × CD7 | CD7 | Dual |
| 175 | BCMA | — | Mono |
| 176 | CS1 | — | Mono |
| 177 | CD19 | — | Mono |
| 178 | CD38 | — | Mono |
| 179 | CS1 | CS1 | Mono |
| 180 | CD38 | CD38 | Mono |
| 181 | APRIL | — | Mono |
| 182 | BCMA × CS1 | — | Tandem |
| 183 | BCMA × CS1 | CS1 | Tandem |
| 184 | BCMA × CD19 | — | Tandem |
| 185 | BCMA × CD38 | — | Tandem |
| 186 | BCMA × CD38 | CD38 | Tandem |
| 187 | CS1 × CD19 | — | Tandem |
| 188 | CS1 × CD19 | CS1 | Tandem |
| 189 | CS1 × CD38 | — | Tandem |
| 190 | CS1 × CD38 | CS1 | Tandem |
| 191 | CS1 × CD38 | CD38 | Tandem |
| 192 | CS1 × CD38 | CS1 and CD38 | Tandem |
| 193 | CD19 × CD38 | — | Tandem |
| 194 | CD19 × CD38 | CD38 | Tandem |
| 195 | APRIL × CS1 | — | Tandem |
| 196 | APRIL × CS1 | CS1 | Tandem |
| 197 | APRIL × BCMA | — | Tandem |
| 198 | APRIL × CD19 | — | Tandem |
| 199 | APRIL × CD38 | — | Tandem |
| 200 | APRIL × CD38 | CD38 | Tandem |
| 201 | BCMA | — | Dual |
| 202 | CS1 | — | Dual |
| 203 | CD19 | — | Dual |
| 204 | CD38 | — | Dual |
| 205 | BCMA | — | Dual |
| 206 | CS1 | CS1 | Dual |
| 207 | CD38 | CD38 | Dual |
| 208 | BCMA × CS1 | — | Dual |
| 209 | BCMA × CS1 | CS1 | Dual |
| 210 | BCMA × CD19 | — | Dual |
| 211 | BCMA × CD38 | — | Dual |
| 212 | BCMA × CD38 | CD38 | Dual |
| 213 | CS1 × CD19 | — | Dual |
| 214 | CS1 × CD19 | CS1 | Dual |
| 215 | CS1 × CD38 | — | Dual |
| 216 | CS1 × CD38 | CS1 | Dual |
| 217 | CS1 × CD38 | CD38 | Dual |
| 218 | CS1 × CD38 | CS1 and CD38 | Dual |
| 219 | CD19 × CD38 | — | Dual |
| 220 | CD19 × CD38 | CD38 | Dual |
| 221 | APRIL × CS1 | — | Dual |
| 222 | APRIL × CS1 | CS1 | Dual |
| 223 | APRIL × BCMA | — | Dual |
| 224 | APRIL × CD19 | — | Dual |
| 225 | APRIL × CD38 | — | Dual |
| 226 | APRIL × CD38 | CD38 | Dual |

E. Mono iNKT-CAR Cells

In some aspects, CAR-bearing immune cells that can be used with the present disclosure comprises iNKT-CAR cells. As used herein, the term “iNKT-CAR cells” or “CAR-iNKT cells) refer to invariant natural killer T (iNKT) cells that express a chimeric antigen receptor. Non-limiting examples of iNKT-CAR cells that can be constructed and used with the present disclosure are further described below.

In some aspects, the disclosure provides an engineered iNKT cell comprising a single CAR, that specifically binds CD7, wherein the iNKT cell is deficient in CD7 (e.g., CD7-iNKT-CARΔCD7 cell). In non-limiting examples, the deficiency in CD7 resulted from (a) modification of CD7 expressed by the iNKT cell such that the chimeric antigen receptors no longer specifically binds the modified CD7, (b) modification of the iNKT cell such that expression of CD7 is reduced in the iNKT cell by at least about 10%, at least about 20%, at least about 30%, at least about 40%, at least about 50%, at least about 60%, at least about 70%, at least about 80%, at least about 90% or more, or (c) modification of the iNKT cell such that CD7 is not expressed (e.g., by deletion or disruption of the gene encoding CD7. In some aspects, the iNKT cell comprises a suicide gene. In non-limiting examples the suicide gene expressed in the CD7-iNKT-CARΔCD7 cells encodes a modified Human-Herpes Simplex Virus-1-thymidine kinase (TK) gene fused in-frame to the extracellular and transmembrane domains of the human CD34 cDNA.

The CAR for a CD7 specific iNKT-CAR cell can be generated by cloning a commercially synthesized anti-CD7 single chain variable fragment (scFv) into a 3rd generation CAR backbone with CD28 and 4-1BB internal signaling domains. An extracellular hCD34 domain can be added after a P2A peptide to enable both detection of CAR following viral transduction and purification using anti-hCD34 magnetic beads. A similar method can be followed for making CARs specific for other malignant T cell antigens.

F. Dual iNKT-CAR Cells

In some aspects, CAR-bearing immune cells that are useful for the present disclosure include dual iNKT-CAR cells. As used herein, the term “dual iNKT-CAR cells” or “iNKT-dCAR” refer to iNKT-CAR cells that express two distinct chimeric antigen receptor polypeptides with affinity to different target antigens expressed within the same effector cell, wherein each CAR functions independently. The CAR can be expressed from a single polynucleotide sequence or multiple polynucleotide sequences. Non-limiting examples of such CAR-bearing immune cells are further described below.

In some aspects, the disclosure provides an engineered iNKT cell comprising a dual CAR (dCAR), i.e., two CARs expressed from a single lentivirus construct, that specifically binds CD7 and CD2, wherein the iNKT cell is deficient in CD7 and CD2 (e.g., CD7×CD2-iNKT-dCARΔCD7ΔCD2 cell). In non-limiting examples, the deficiency in CD7 and CD2 resulted from (a) modification of CD7 and CD2 expressed by the iNKT cell such that the chimeric antigen receptors no longer specifically binds the modified CD7 or CD2, (b) modification of the iNKT cell such that expression of CD7 and CD2 is reduced in the iNKT cell by at least about 10%, at least about 20%, at least about 30%, at least about 40%, at least about 50%, at least about 60%, at least about 70%, at least about 80%, at least about 90% or more, or (c) modification of the iNKT cell such that CD7 and CD2 is not expressed (e.g., by deletion or disruption of the gene encoding CD7 and/or CD2. In some aspects, the iNKT cell comprises a suicide gene. In non-limiting examples the suicide gene expressed in the CD7*CD2-iNKT-dCARΔCD7ΔCD2 cells encodes a modified Human-Herpes Simplex Virus-1-thymidine kinase (TK) gene fused in-frame to the extracellular and transmembrane domains of the human CD34 cDNA.

G. Tandem iNKT-CAR Cells

In some aspects, CAR-bearing immune cells that are useful for the present disclosure comprise tandem iNKT-CAR cells. As used herein, the term “tandem iNKT-CAR cells” or “iNKT-tCAR” refer to iNKT-CAR cells with a single chimeric antigen polypeptide containing two distinct antigen recognition domains with affinity to different targets, wherein the antigen recognition domains are linked through a peptide linker and share common costimulatory domain(s), and wherein binding of either antigen recognition domain will signal though a common costimulatory domains(s) and signaling domain. Non-limiting examples of such CAR-bearing immune cells are further described below.

In some aspects, the disclosure provides an engineered iNKT cell comprising a tandem CAR (tCAR), i.e., two scFv sharing a single intracellular domain, that specifically binds CD7 and CD2, wherein the iNKT cell is deficient in CD7 and CD2 (e.g., CD7×CD2-iNKT-tCARΔCD7ΔCD2 cell). In non-limiting examples, the deficiency in CD7 and CD2 resulted from (a) modification of CD7 and CD2 expressed by the iNKT cell such that the chimeric antigen receptors no longer specifically binds the modified CD7 or CD2, (b) modification of the iNKT cell such that expression of CD7 and CD2 is reduced in the iNKT cell by at least about 10%, at least about 20%, at least about 30%, at least about 40%, at least about 50%, at least about 60%, at least about 70%, at least about 80%, at least about 90% or more, or (c) modification of the iNKT cell such that CD7 and CD2 is not expressed (e.g., by deletion or disruption of the gene encoding CD7 and/or CD2. In some aspects, the iNKT cell comprises a suicide gene. In non-limiting examples, the suicide gene expressed in the CD7*CD2-iNKT-tCARΔCD7ΔCD2 cells encodes a modified Human-Herpes Simplex Virus-1-thymidine kinase (TK) gene fused in-frame to the extracellular and transmembrane domains of the human CD34 cDNA.

A tCAR for a genome-edited, tandem iNKT-CAR cell, i.e., CD7*CD2-iNKT-tCARΔCD7ΔCD2, can be generated by cloning a commercially synthesized anti-CD7 single chain variable fragment (scFv) and an anti-CD2 single chain variable fragment (scFv) into a 3rd generation CAR backbone with CD28 and 4-1BB internal signaling domains. An extracellular hCD34 domain can be added after a P2A peptide to enable both detection of CAR following viral transduction and purification using anti-hCD34 magnetic beads. A similar method can be followed for making tCARs specific for other malignant T cell antigens.

Non-limiting examples of iNKT-CAR cells (e.g., mono iNKT-CAR cells, genome-edited iNKT-CAR cells, dual iNKT-CAR cells, or tandem iNKT-CAR cells) that can be constructed and used with the present disclosure are provided in Tables 1 and 1.

### V. Transgenic TCR-Bearing Immune Cells

As described herein, modified immune cells other than CAR-bearing immune cells can also be used in combination with an IL-7 protein disclosed herein, e.g., to treat a solid tumor. For example, in some aspects, a method of treating a solid tumor disclosed herein comprises administering a population of transgenic TCR-bearing immune cells in combination with an IL-7 protein.

In some aspects, the transgenic (TCR)-bearing immune cells can specifically target a solid tumor antigen. In some aspects, the solid tumor antigen is selected from mesothelin, MR1, guanylate cyclase C (GC-C), epidermal growth factor receptor (EGFR or erbB-1), human epidermal growth factor receptor 2 (HER2 or erbB2), erbB-3, erbB-4, MUC-1, melanoma-associated chondroitin sulfate proteoglycan (MCSP), folate receptor 1 (FOLR1), CD4, CD19, CD20, CD22, CD30, CD33, CD38, CD44, CD44v6, CD44v7/8, CD70, CD123, CD138, CD171, CEA, CSPG4, CXCR5, c-Met, HERV-envelope protein, eriostin, Bigh3, SPARC, BCR, CD79, CD37, EGFRvIII, EGP2, EGP40, IGFr, L1CAM, AXL, Tissue Factor (TF), CD74, EpCAM, EphA2, MRP3cadherin 19 (CDH19), epidermal growth factor 2 (HER2), 5T4, 8H9, αᵥβ₆ integrin, BCMA, B7-H3, B7-H6, CAIX, CA9, FAP, FBP, fetal AchR, FRcc, GD2, GD3, Glypican-1 (GPC1), Glypican-2 (GPC2), Glypican-3 (GPC3), HLA-A1+MAGE1, HLA-A1+NY-ESO-1, IL-13Rcc2, Lewis-Y, KDR, MCSP, Muc1, Muc16, NCAM, NKG2D ligands, NY-ESO-1, PRAMS, PSC1, PSCA, PSMA, ROR1, ROR2, SP17, surviving, TAG72, TEMs, carcinoembryonic antigen, HMW-MAA, VEGF, CLDN18.2, or combinations thereof.

As described herein, in some aspects, transducing an immune cell to express an exogenous transgene can comprise the use of a vector (e.g., lentiviral or retroviral) expressing the transgene. However, for transgenes such as those encoding a transgenic TCR, lentiviral or retroviral insertion of the transgenes can generate random integration events at multiple loci and thus, resulting in supra-physiological expression levels. For transgenic TCRs, mispairing with endogenous TCRs (e.g., where the immune cell being modified is a T cell), if not removed, can also lead to unwanted specificity and low yield. Therefore, as described above for CAR-bearing immune cells (e.g., CAR-T cells or iNKT-CAR cells), one or more genes that are endogenously expressed in the immune cells can be modified (e.g., deleted) so as not to interfere with the expression of the transgene (e.g., encoding a transgenic TCR).

In some aspects, a transgenic TCR-bearing immune cell comprises a T cell. In certain aspects, the T cell is a CD8+ T cell. In certain aspects, the T cell is a CD4+ T cell. In some aspects, the T cell comprises both CD8+ T cells and CD4+ T cells. In certain aspects, the T cell expressing the transgenic TCR is deficient in endogenous T cell receptor (TCR) signaling as a result of deleting a part of the T Cell Receptor (TCR)-CD3 complex. In some aspects, decreasing or eliminating endogenous TCR signaling in the T cells can prevent or reduce graft versus host disease (GvHD) when allogenic T cells are used to produce the transgenic TCR-bearing immune cells. In some aspects, decreasing or eliminating endogenous TCR signaling in the T cells can help increase the expression of the transgenic TCR in the cell. Methods for eliminating or suppressing endogenous TCR signaling are known in the art and include, but are not limited to, deleting a part of the TCR-CD3 receptor complex, e.g., the TCR receptor alpha chain (TRAC), the TCR receptor beta chain (TRBC), CD3.epsilon, CD3.gamma, CD3.delta, and/or CD3.gamma.

In some aspects, the TCR-bearing immune cells are autologous cells, i.e., an immune cell or cells taken from a subject who is in need of a combination therapy disclosed herein. In such aspects, the immune cells isolated from the subject can be modified to express a transgenic TCR and then reintroduced into the subject. Autologous cells have the advantage of avoiding any immunologically-based rejection of the cells. Alternatively, the cells can be allogeneic, e.g., taken from a donor. Typically, when the cells come from a donor, they will be from a donor who is sufficiently immunologically compatible with the recipient, i.e., will not be subject to transplant rejection, to lessen or remove the need for immunosuppression. In some aspects, the cells are taken from a xenogeneic source, i.e., a non-human mammal that has been genetically engineered to be sufficiently immunologically compatible with the recipient, or the recipient's species. Methods for determining immunological compatibility are known in the art, and include tissue typing to assess donor-recipient compatibility for HLA and ABO determinants. See, e.g., Transplantation Immunology, Bach and Auchincloss, Eds. (Wiley, John & Sons, Incorporated 1994).

In some aspects, the transgenic TCR-bearing immune cells disclosed herein recognize the solid tumor antigens in an HLA-independent manner. In some aspects, the transgenic TCR-bearing immune cells are capable of selectively killing solid tumors (i.e., do not kill healthy cells, even healthy, stressed, or infected healthy cells). In some aspects, the transgenic TCR-bearing immune cells recognize monomorphic MHC class 1-related protein MR1. Examples of such TCR-bearing immune cells are provided in Crowther, M. D., et al., Nat Immunol 21(2): 178-185 (February 2020) and US 2019/0389926 A1, each of which is incorporated herein by reference in its entirety.

### VI. Chimeric Antigen Receptor and Transgenic T-Cell Receptor Construction

CARs and/or transgenic TCRs useful for the present disclosure can be designed by any appropriate methods known in the art. See, e.g., WO2018027036A1 and WO2019138217A1, each of which is incorporated herein by reference in its entirety. Lentiviral vectors and cell lines can be obtained, and guide RNAs designed, validated, and synthesized, as disclosed therein as well as by methods known in the art and from commercial sources.

Engineered CARs and/or transgenic TCRs can be introduced into immune cells (e.g., T cells or iNKT cells) using retroviruses, which efficiently and stably integrate a nucleic acid sequence encoding the chimeric antigen receptor or the transgenic TCR into the target cell genome. For instance, T-cell receptor (TCR) is a molecule found on the surface of T cells which is responsible for recognizing fragments of target antigen as peptides bound to major histocompatibility complex (MHC) molecules. The TCR is a heterodimer composed of two different protein chains. In humans, in 95% of T cells the TCR consists of an alpha (α) chain and a beta (β) chain (encoded by IRA and TRB, respectively), whereas in 5% of T cells the TCR consists of gamma and delta (γ/δ) chains (encoded by TRG and TRD, respectively). Accordingly, in some aspects, a transgenic TCR-bearing immune cell disclosed herein can be constructed by artificially introducing (e.g., via a vector) the TRA and TRB genes or the TRG and TRD genes into immune cells. In certain aspects, the TRA and TRB genes or the TRG and TRD genes can be expressed in the same vector. In some aspects, TRA and TRB genes or the TRG and TRD genes are expressed in separate vectors.

Other methods known in the art include, but are not limited to, lentiviral transduction, transposon-based systems, direct RNA transfection, and CRISPR/Cas systems (e.g., type I, type II, or type Ill systems using a suitable Cas protein such Cas3, Cas4, Cas5, Cas5e (or CasD), Cas6, Cas6e, Cas6f, Cas7, Cas8a1, Cas8a2, Cas8b, Cas8c, Cas9, Cas10, Casl Od, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966, etc.). Zinc finger nucleases (ZFNs) and transcription activator-like effector nucleases (TALENs) can also be used. See, e.g., Shearer RF and Saunders DN, “Experimental design for stable genetic manipulation in mammalian cell lines: lentivirus and alternatives,” Genes Cells 2015 January; 20(1): 1-10.

### VII. Nucleic Acids, Vectors, Host Cells

Further aspect described herein pertains to one or more nucleic acid molecules that encode a therapeutic agent described herein (e.g., an IL-7 protein, CARs, and/or transgenic TCRs). The nucleic acids can be present in whole cells, in a cell lysate, or in a partially purified or substantially pure form. A nucleic acid is “isolated” or “rendered substantially pure” when purified away from other cellular components or other contaminants, e.g., other cellular nucleic acids (e.g., other chromosomal DNA, e.g., the chromosomal DNA that is linked to the isolated DNA in nature) or proteins, by standard techniques, including alkaline/SDS treatment, CsCl banding, column chromatography, restriction enzymes, agarose gel electrophoresis and others well known in the art. See, F. Ausubel, et al., ed. (1987) Current Protocols in Molecular Biology, Greene Publishing and Wiley Interscience, New York. A nucleic acid described herein can be, for example, DNA or RNA and can or cannot contain intronic sequences. In a certain aspects, the nucleic acid is a cDNA molecule. Nucleic acids described herein can be obtained using standard molecular biology techniques known in the art.

Certain nucleic acid molecules disclosed herein are those encoding an IL-7 protein (e.g., disclosed herein). Exemplary nucleic acid sequences encoding an IL-7 protein disclosed herein are set forth in SEQ ID NOs: 29-39.

In some aspects, the present disclosure provides a vector comprising an isolated nucleic acid molecule encoding a therapeutic agent disclosed herein (e.g., an IL-7 protein). In some aspects, a vector can be used for gene therapy.

When used as a gene therapy (e.g., in humans), a nucleic acid encoding a therapeutic agent disclosed herein (e.g., an IL-7 protein) can be administered at a dosage in the range of 0.1 mg to 200 mg. In certain aspects, the dosage is in the range of 0.6 mg to 100 mg. In certain aspects, the dosage is in the range of 1.2 mg to 50 mg.

Suitable vectors for the disclosure include expression vectors, viral vectors, and plasmid vectors. In some aspects, the vector is a viral vector.

As used herein, an expression vector refers to any nucleic acid construct which contains the necessary elements for the transcription and translation of an inserted coding sequence, or in the case of an RNA viral vector, the necessary elements for replication and translation, when introduced into an appropriate host cell. Expression vectors can include plasmids, phagemids, viruses, and derivatives thereof.

As used herein, viral vectors include, but are not limited to, nucleic acid sequences from the following viruses: retrovirus, such as Moloney murine leukemia virus, Harvey murine sarcoma virus, murine mammary tumor virus, and Rous sarcoma virus; lentivirus; adenovirus; adeno-associated virus; SV40-type viruses; polyomaviruses; Epstein-Barr viruses; papilloma viruses; herpes virus; vaccinia virus; polio virus; and RNA virus such as a retrovirus. One can readily employ other vectors well-known in the art. Certain viral vectors are based on non-cytopathic eukaryotic viruses in which non-essential genes have been replaced with the gene of interest. Non-cytopathic viruses include retroviruses, the life cycle of which involves reverse transcription of genomic viral RNA into DNA with subsequent proviral integration into host cellular DNA.

In some aspects, a vector is derived from an adeno-associated virus. In some aspects, a vector is derived from a lentivirus. Examples of the lentiviral vectors are disclosed in WO9931251, WO9712622, WO9817815, WO9817816, and WO9818934, each which is incorporated herein by reference in its entirety.

Other vectors include plasmid vectors. Plasmid vectors have been extensively described in the art and are well-known to those of skill in the art. See, e.g., Sambrook et al., Molecular Cloning: A Laboratory Manual, Second Edition, Cold Spring Harbor Laboratory Press, 1989. In the last few years, plasmid vectors have been found to be particularly advantageous for delivering genes to cells in vivo because of their inability to replicate within and integrate into a host genome. These plasmids, however, having a promoter compatible with the host cell, can express a peptide from a gene operably encoded within the plasmid. Some commonly used plasmids available from commercial suppliers include pBR322, pUC18, pUC19, various pcDNA plasmids, pRC/CMV, various pCMV plasmids, pSV40, and pBlueScript. Additional examples of specific plasmids include pcDNA3.1, catalog number V79020; pcDNA3.1/hygro, catalog number V87020; pcDNA4/myc-His, catalog number V86320; and pBudCE4.1, catalog number V53220, all from Invitrogen (Carlsbad, Calif.). Other plasmids are well-known to those of ordinary skill in the art. Additionally, plasmids can be custom designed using standard molecular biology techniques to remove and/or add specific fragments of DNA.

Also encompassed by the present disclosure is a method for making a therapeutic agent disclosed herein (e.g., an IL-7 protein). In some aspects, such a method can comprise expressing the therapeutic agent (e.g., an IL-7 protein) in a cell comprising a nucleic acid molecule encoding the therapeutic agent, e.g., SEQ ID NOs: 29-39. Additional details regarding the method for making an IL-7 protein disclosed herein are provided, e.g., in WO 2016/200219, which is herein incorporated by reference in its entirety. Host cells comprising these nucleotide sequences are encompassed herein. Non-limiting examples of host cell that can be used include immortal hybridoma cell, NS/0 myeloma cell, 293 cell, Chinese hamster ovary (CHO) cell, HeLa cell, human amniotic fluid-derived cell (CapT cell), COS cell, or combinations thereof.

### VIII. Pharmaceutical Compositions

Further provided herein are compositions comprising one or more therapeutic agents (e.g., an IL-7 protein and/or population of modified immune cells disclosed herein, e.g., CAR-bearing immune cells and/or transgenic TCR-bearing immune cells) having the desired degree of purity in a physiologically acceptable carrier, excipient or stabilizer (Remington's Pharmaceutical Sciences (1990) Mack Publishing Co., Easton, Pa.). In some aspects, a composition disclosed herein comprises an IL-7 protein or a population of modified immune cells (e.g., CAR-bearing immune cells and/or transgenic TCR-bearing immune cells). As disclosed herein, such compositions can be used in combination (e.g., a first composition comprising an IL-7 protein and a second composition comprising a population of modified immune cells (e.g., CAR-bearing immune cells and/or transgenic TCR-bearing immune cells)). In some aspects, a composition disclosed herein can comprise both an IL-7 protein and a population of modified immune cells (e.g., CAR-bearing immune cells and/or transgenic TCR-bearing immune cells).

Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN®, PLURONICS® or polyethylene glycol (PEG).

In some aspects, a composition disclosed herein comprises one or more additional components selected from: a bulking agent, stabilizing agent, surfactant, buffering agent, or combinations thereof.

Buffering agents useful for the current disclosure can be a weak acid or base used to maintain the acidity (pH) of a solution near a chosen value after the addition of another acid or base. Suitable buffering agents can maximize the stability of the pharmaceutical compositions by maintaining pH control of the composition. Suitable buffering agents can also ensure physiological compatibility or optimize solubility. Rheology, viscosity and other properties can also dependent on the pH of the composition. Common buffering agents include, but are not limited to, a Tris buffer, a Tris-Cl buffer, a histidine buffer, a TAE buffer, a HEPES buffer, a TBE buffer, a sodium phosphate buffer, a MES buffer, an ammonium sulfate buffer, a potassium phosphate buffer, a potassium thiocyanate buffer, a succinate buffer, a tartrate buffer, a DIPSO buffer, a HEPPSO buffer, a POPSO buffer, a PIPES buffer, a PBS buffer, a MOPS buffer, an acetate buffer, a phosphate buffer, a cacodylate buffer, a glycine buffer, a sulfate buffer, an imidazole buffer, a guanidine hydrochloride buffer, a phosphate-citrate buffer, a borate buffer, a malonate buffer, a 3-picoline buffer, a 2-picoline buffer, a 4-picoline buffer, a 3,5-lutidine buffer, a 3,4-lutidine buffer, a 2,4-lutidine buffer, a Aces, a diethylmalonate buffer, a N-methylimidazole buffer, a 1,2-dimethylimidazole buffer, a TAPS buffer, a bis-Tris buffer, a L-arginine buffer, a lactate buffer, a glycolate buffer, or combinations thereof.

In some aspects, a composition disclosed herein further comprises a bulking agent. Bulking agents can be added to a pharmaceutical product in order to add volume and mass to the product, thereby facilitating precise metering and handling thereof. Bulking agents that can be used with the present disclosure include, but are not limited to, sodium chloride (NaCl), mannitol, glycine, alanine, or combinations thereof.

In some aspects, a composition disclosed herein can also comprise a stabilizing agent. Non-limiting examples of stabilizing agents that can be used with the present disclosure include: sucrose, trehalose, raffinose, arginine, or combinations thereof.

In some aspects, a composition disclosed herein comprises a surfactant. In certain aspects, the surfactant can be selected from the following: alkyl ethoxylate, nonylphenol ethoxylate, amine ethoxylate, polyethylene oxide, polypropylene oxide, fatty alcohols such as cetyl alcohol or oleyl alcohol, cocamide MEA, cocamide DEA, polysorbates, dodecyl dimethylamine oxide, or combinations thereof. In some aspects, the surfactant is polysorbate 20 or polysorbate 80.

In some aspects, a composition comprising an IL-7 protein can be formulated using the same formulation used for the population of modified immune cells (e.g., CAR-bearing immune cells and/or transgenic TCR-bearing immune cells) (e.g., which is to be used in combination with the IL-7 protein). In some aspects, an IL-7 protein and a population of modified immune cells (e.g., CAR-bearing immune cells and/or transgenic TCR-bearing immune cells) are formulated using different formulations.

In some aspects, an IL-7 protein disclosed herein is formulated in a composition comprising (a) a basal buffer, (b) a sugar, and (c) a surfactant. In certain aspects, the basal buffer comprises histidine-acetate or sodium citrate. In some aspects, the basal buffer is at a concentration of about 10 to about 50 nM. In some aspects, a sugar comprises sucrose, trehalose, dextrose, or combinations thereof. In some aspects, the sugar is present at a concentration of about 2.5 to about 5.0 w/v %. In certain aspects, the surfactant is selected from polysorbate, polyoxyethylene alkyl ether, polyoxyethylene stearate, alkyl sulfates, polyvinyl pyridone, poloxamer, or combinations thereof. In some aspects, the surfactant is at a concentration of about 0.05% to about 6.0 w/v %.

In some aspects, a composition disclosed herein (e.g., comprising an IL-7 protein and/or population of modified immune cells, e.g., CAR-bearing immune cells and/or transgenic TCR-bearing immune cells) further comprises an amino acid. In certain aspects, the amino acid is selected from arginine, glutamate, glycine, histidine, or combinations thereof. In certain aspects, the composition further comprises a sugar alcohol. Non-limiting examples of sugar alcohol includes: sorbitol, xylitol, maltitol, mannitol, or combinations thereof.

In some aspects, an IL-7 protein disclosed herein is formulated in a composition comprising the following: (a) sodium citrate (e.g., about 20 mM), (b) sucrose (e.g., about 5%), (c) sorbitol (e.g., about 1.5%), and (d) Tween 80 (e.g., about 0.05%).

In some aspects, an IL-7 protein of the present disclosure is formulated as described in WO 2017/078385 A1, which is incorporated herein in its entirety.

A pharmaceutical composition can be formulated for any route of administration to a subject. Specific examples of routes of administration include intramuscularly, subcutaneously, ophthalmic, intravenously, intraperitoneally, intradermally, intraorbitally, intracerebrally, intracranially, intraspinally, intraventricular, intrathecally, intracistemally, intracapsularly, or intratumorally. Parenteral administration, characterized by either subcutaneous, intramuscular or intravenous injection, is also contemplated herein. In some aspects, an IL-7 protein and a population of modified immune cells (e.g., CAR-bearing immune cells and/or transgenic TCR-bearing immune cells) are administered using the same route of administration. In some aspects, an IL-7 protein and a population of modified immune cells (e.g., CAR-bearing immune cells and/or transgenic TCR-bearing immune cells) are administered using different routes of administration. In some aspects, an IL-7 protein is administered intravenously, subcutaneously, or intratumorally. In some aspects, a population of modified immune cells (e.g., CAR-bearing immune cells and/or transgenic TCR-bearing immune cells) is administered intravenously.

Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungi static concentrations can be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions includes EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions can be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Topical mixtures comprising an antibody are prepared as described for the local and systemic administration. The resulting mixture can be a solution, suspension, emulsions or the like and can be formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

A therapeutic agent described herein (e.g., an IL-7 protein) can be formulated as an aerosol for topical application, such as by inhalation (see, e.g., U.S. Pat. Nos. 4,044,126, 4,414,209 and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflations, alone or in combination with an inert carrier such as lactose. In such a case, the particles of the formulation will, in one aspect, have diameters of less than 50 microns, in one aspect less than 10 microns.

A therapeutic agent disclosed herein (e.g., an IL-7 protein) can be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the antibody alone or in combination with other pharmaceutically acceptable excipients can also be administered.

Transdermal patches, including iontophoretic and electrophoretic devices, are well known to those of skill in the art, and can be used to administer an antibody. For example, such patches are disclosed in U.S. Pat. Nos. 6,267,983, 6,261,595, 6,256,533, 6,167,301, 6,024,975, 6,010715, 5,985,317, 5,983,134, 5,948,433, and 5,860,957, each of which is herein incorporated by reference in its entirety.

In certain aspects, a pharmaceutical composition comprising a therapeutic agent described herein (e.g., an IL-7 protein) is a lyophilized powder, which can be reconstituted for administration as solutions, emulsions and other mixtures. It can also be reconstituted and formulated as solids or gels. The lyophilized powder is prepared by dissolving an antibody or antigen-binding portion thereof described herein, or a pharmaceutically acceptable derivative thereof, in a suitable solvent. In some aspects, the lyophilized powder is sterile. The solvent can contain an excipient which improves the stability or other pharmacological component of the powder or reconstituted solution, prepared from the powder. Excipients that can be used include, but are not limited to, dextrose, sorbitol, fructose, corn syrup, xylitol, glycerin, glucose, sucrose or other suitable agent. The solvent can also contain a buffer, such as citrate, sodium or potassium phosphate or other such buffer known to those of skill in the art at, in one aspect, about neutral pH. Subsequent sterile filtration of the solution followed by lyophilization under standard conditions known to those of skill in the art provides the desired formulation. In some aspects, the resulting solution can be apportioned into vials for lyophilization. Each vial can contain a single dosage or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

Compositions provided herein can also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874. In a specific aspect, the combination therapy disclosed herein (i.e., IL-7 protein in combination with a population of CAR-bearing or transgenic TCR-bearing immune cells) can be used treat a solid tumor.

The compositions to be used for in vivo administration can be sterile. This is readily accomplished by filtration through, e.g., sterile filtration membranes.

The following examples are merely illustrative and should not be construed as limiting the scope of this disclosure in any way as many variations and equivalents will become apparent to those skilled in the art upon reading the present disclosure.

### EXAMPLES

### Example 1: Analysis of the Anti-Tumor Effects of Anti-Mesothelin CAR-T Cells and IL-7 Protein Combination Therapy

To assess the efficacy of a combination therapy disclosed herein (i.e., IL-7 protein in combination with a population of CAR-bearing immune cells) in treating solid tumors, a pancreatic tumor mouse model was used. Briefly, non-conditioned NSG mice were injected with GFP-labelled AsPC-1 tumor cells (2×10⁶ cells/mouse). To prepare the CAR-T cells for in vivo administration, T cells were isolated/purified from peripheral blood mononuclear cells (PBMCs) harvested from healthy donors. The T cells were then activated in vitro using anti-CD3 and anti-CD28 antibodies. Approximately two days later, the TCR receptor alpha chain (TRAC) was deleted using CRISPR/Cas9 system. The next day, the T cells were transduced with CARs targeting Mesothelin. The transduced T cells were allowed to further expand for approximately a week. Residual TCR+ T cells, that escaped gene editing, were removed by magnetic depletion, and then, the transduced T cells (i.e., CD3− CAR+) were isolated and administered to the animals.

Approximately fifteen days after tumor cell injection, the animals received a dose of anti-mesothelin CAR-T cells (5×10⁵ cells/mouse) intravenously. About 24 hours later, the animals were further treated with either vehicle or a modified IL-7 protein (e.g., disclosed herein). The IL-7 protein was administered to the animals at a dose of 10 mg/kg for a total of three doses (at days 1, 15, and 29 post CAR-T cell administration). The different treatment groups are shown in Table 3 (below). The animals were monitored on a weekly basis for both tumor size and survival.

TABLE 3
Treatment Groups

| | | Number of |
|---|---|---|
| Group | Treatment | Animals |
| Control | Buffer + Vehicle (“Tumor only”) | 5 |
| 1 | IL-7 + Vehicle (“Tumor + IL7”) | 5 |
| 2 | Buffer + CAR-T cells (“Tumor + | 5 |
| | MesoCAR”) | |
| 3 | IL-7 + CAR-T cells (“Tumor + | 5 |
| | MesoCAR + IL-7”) | |

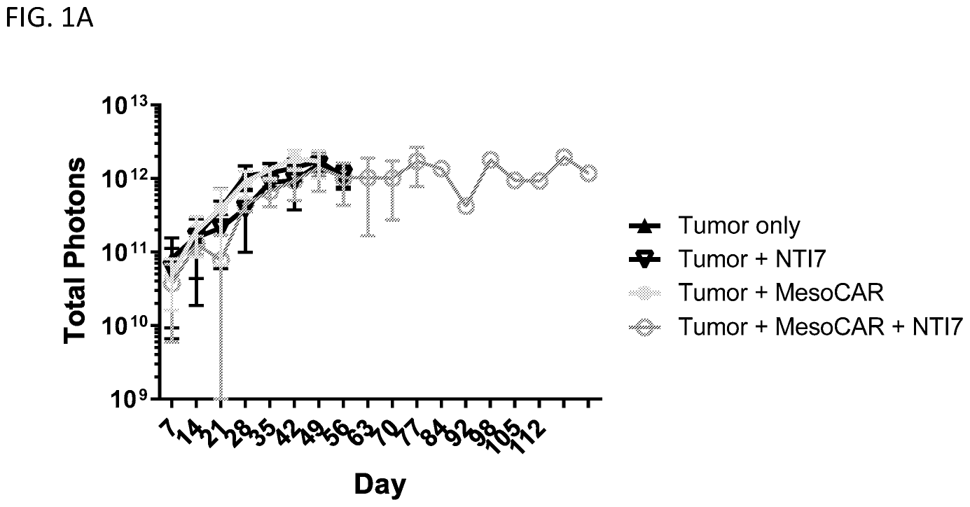

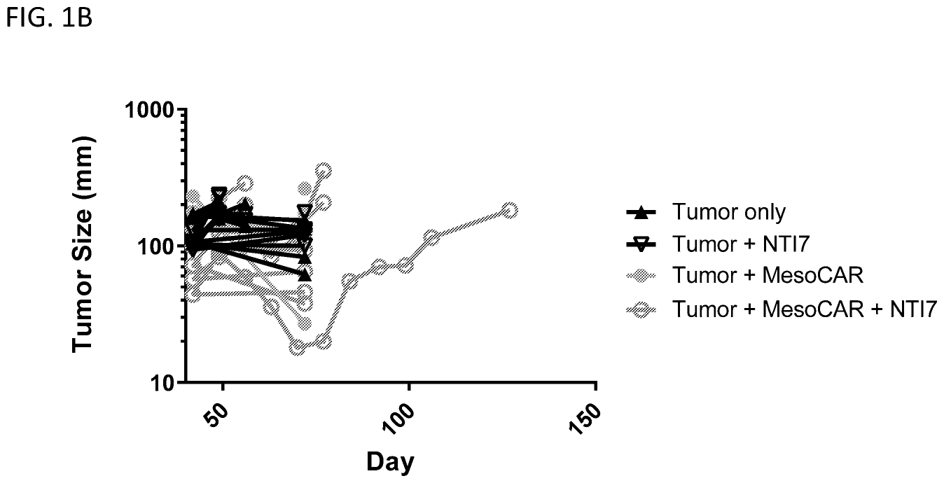

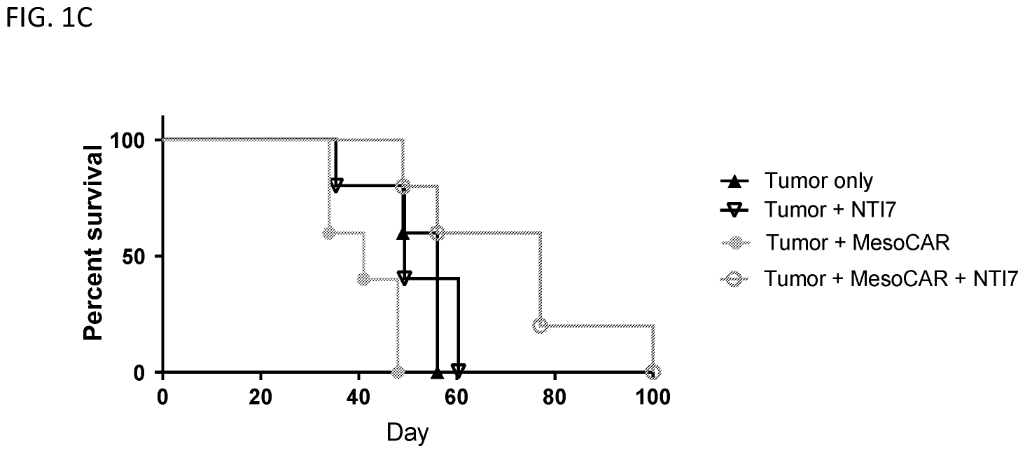

As shown in FIGS. 1A and 1B, there did not appear to be an overall significant reduction in tumor volume among the different treatment groups. At least one of the animals treated with the combination of both IL-7 protein and CAR-T cells did exhibit significant reduction in tumor volume until about 75-80 days post CAR-T cell administration (see FIG. 1B). Despite the lack of significant reduction in tumor volume, treatment of the animals with IL-7 protein in combination with CAR-T cells did increase overall survival compared to the other treatment groups.

### Example 2: Further Analysis of the Anti-Tumor Effects of Anti-Mesothelin CAR-T Cells and IL-7 Protein Combination Therapy

To further assess the efficacy of a combination therapy disclosed herein (i.e., IL-7 protein in combination with a population of CAR-bearing immune cells) in treating solid tumors, the above Example was repeated as follows. Briefly, non-conditioned NSG mice were injected with GFP-labelled AsPC-1 tumor cells (1.5×10⁶ cells/mouse). To prepare the CAR-T cells for in vivo administration, T cells were isolated/purified from peripheral blood mononuclear cells (PBMCs) harvested from healthy donors. The T cells were then activated in vitro using anti-CD3 and anti-CD28 antibodies. The next day the T cells were transduced with anti-mesothelin CARs (i.e., same as those used in Example 1). The following-day the TCR receptor alpha chain (TRAC) was deleted using CRISPR/Cas9 system. The transduced T cells were allowed to further expand for approximately a week. Residual TCR+ T cells, that escaped gene editing, were removed by magnetic depletion and then, the transduced T cells (i.e., CD3− CAR+) were isolated and administered to the animals.

Seven days after subcutaneous tumor cell injection, the animals received a dose of anti-mesothelin CAR-T cells (1×10⁶ cells/mouse) intravenously. About 24 hours later, the animals were further treated with either vehicle or a modified IL-7 protein (e.g., disclosed herein). The IL-7 protein was administered to the animals at a dose of 10 mg/kg every other week for a total of three doses. The different treatment groups are the same as in Example 1 (see Table 3). The animals were monitored on a weekly basis for both tumor size and survival.

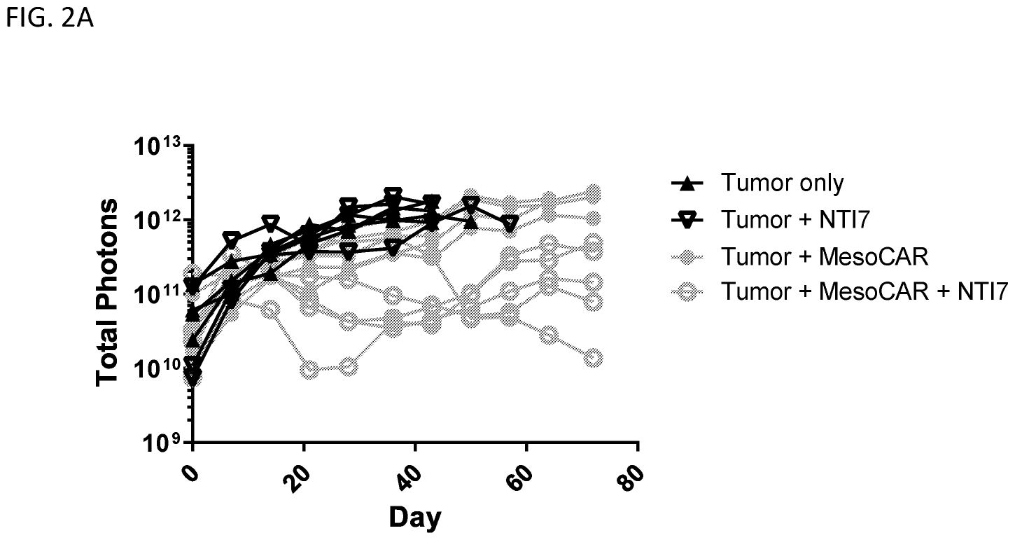

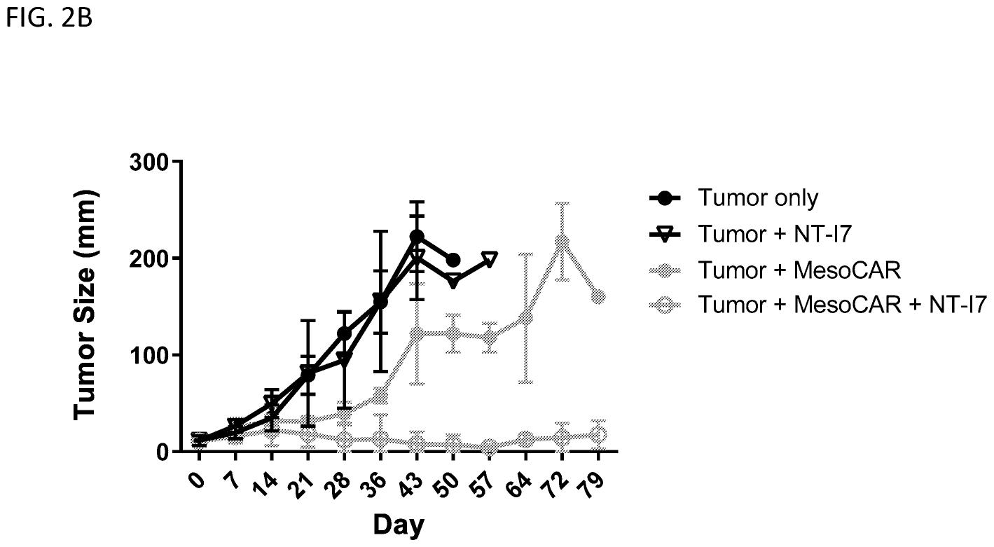

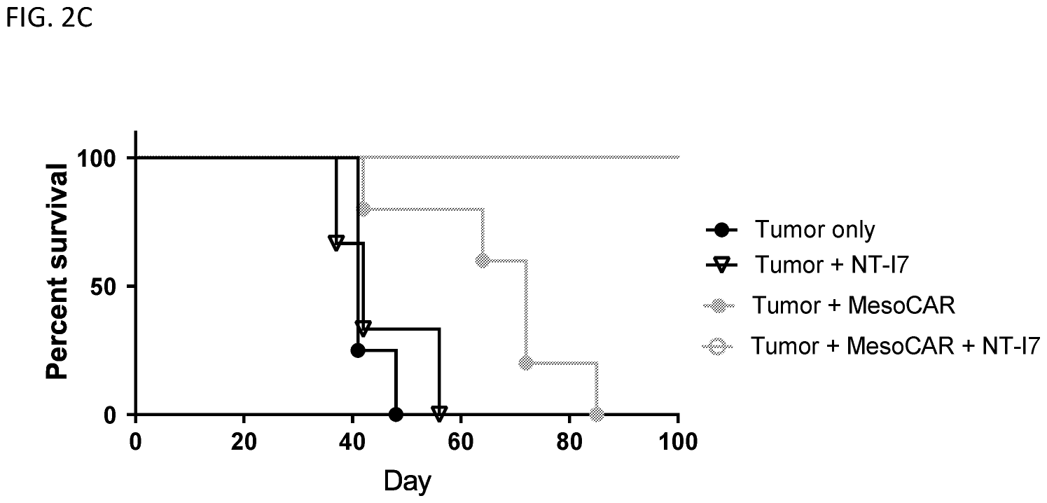

In contrast to Example 1, animals treated with the combination of IL-7 protein and CAR-T cells did exhibit a significant reduction in tumor volume compared to the other treatment groups (see FIGS. 2A and 2B). The reduction in tumor volume was noticeable as early as about 2 weeks post CAR-T cell administration. As shown in FIG. 2C, the reduction in tumor volume was associated with increased survival. Compared to the other treatment groups, all the animals that received the combination therapy (i.e., IL-7 protein in combination with a population of CAR-T cells) survived the entire duration of the experiment (i.e., 100 days post CAR-T cell administration).

Collectively, the above results demonstrate that IL-7 proteins disclosed herein can greatly improve the anti-tumor effects of CAR-T cells. Accordingly, the above results suggest that the combination therapy disclosed herein (i.e., IL-7 protein in combination with a population of modified immune cells, e.g., CAR-bearing immune cells and/or transgenic TCR-bearing immune cells) can be effective in treating solid tumors.

1. A method of treating a solid tumor in a subject in need thereof, comprising administering to the subject a population of immune cells in combination with an interleukin-7 (IL-7) protein, which is conjugated to a half-life extending moiety (IL-7 fusion protein), wherein the population of immune cells comprise chimeric antigen receptor (CAR)-bearing immune cells, transgenic T-cell receptor (TCR)-bearing immune cells, or both.

2. The method of claim 1, wherein, after the administering, the subject exhibits one or more of the following as compared to a reference subject: (a) a reduced tumor volume, (b) an increased survival, or (c) both (a) and (b), wherein the reference subject comprises the subject prior to the administering and/or a corresponding subject that received an administration of the IL-7 fusion protein or the population of immune cells alone.

3-5. (canceled)

6. A method of enhancing an anti-tumor activity of a population of immune cells in a subject having a solid tumor, comprising administering to the subject the population of immune cells in combination with an interleukin-7 (IL-7) protein, which is conjugated to a half-life extending moiety (IL-7 fusion protein), wherein the population of immune cells comprise chimeric antigen receptor (CAR)-bearing immune cells, transgenic T-cell receptor (TCR)-bearing immune cells, or both, and wherein the IL-7 fusion protein is capable of enhancing the anti-tumor activity of the population of immune cells.

7-9. (canceled)

10. A method of increasing an expansion, persistence, and/or survival of a population of immune cells in a subject having a solid tumor, comprising administering to the subject the population of immune cells in combination with an interleukin-7 (IL-7) protein, which is conjugated to a half-life extending moiety (IL-7 fusion protein), wherein the population of immune cells comprise chimeric antigen receptor (CAR)-bearing immune cells, transgenic T-cell receptor (TCR)-bearing immune cells, or both, and wherein the IL-7 fusion protein is capable of enhancing the expansion, persistence, and/or survival of the population of immune cells.

11-13. (canceled)

14. The method of claim 1, wherein the IL-7 fusion protein is administered: (a) at a dose of between about 20 μg/kg and about 2,000 μg/kg, (b) at a dosing interval of at least about once a week, or (c) both (a) and (b).

15-16. (canceled)

17. The method of claim 14, wherein the IL-7 fusion protein is administered at a dose of about 60 μg/kg, about 120 μg/kg, about 240 μg/kg, about 360 μg/kg, about 480 μg/kg, about 600 μg/kg, or about 720 μg/kg.

18. (canceled)

19. The method of claim 1, wherein the population of CAR-bearing immune cells is administered at a dose of less than about 100,000 CAR-bearing immune cells per kilogram of the subject's body weight.

20-24. (canceled)

25. The method of claim 1, wherein the IL-7 fusion protein and the population of immune cells are administered concurrently or sequentially.

26-27. (canceled)

28. The method of claim 25, wherein the IL-7 fusion protein is administered to the subject after administering the population of immune cells.

29-31. (canceled)

32. The method of claim 1, wherein the IL-7 fusion protein further comprises an oligopeptide consisting of 1 to 10 amino acid residues.

33-35. (canceled)

36. The method of claim 1, wherein the half-life extending moiety comprises an Fc, albumin, an albumin-binding polypeptide, Pro/Ala/Ser (PAS), a C-terminal peptide (CTP) of the β subunit of human chorionic gonadotropin, polyethylene glycol (PEG), long unstructured hydrophilic sequences of amino acids (XTEN), hydroxyethyl starch (HES), an albumin-binding small molecule, or a combination thereof.

37-38. (canceled)

39. The method of claim 1, wherein the IL-7 protein comprises an amino acid sequence having a sequence identity of at least about 70% to the amino acid sequence set forth in any one of SEQ ID NOs: 1-6 and 15-27.

40-41. (canceled)

42. The method of claim 1, wherein the CAR-bearing immune cells, transgenic TCR-bearing immune cells, or both comprise an antigen-binding domain, which binds to: (a) a solid tumor antigen, (b) one or more antigens selected from CD2, CD3ε, CD4, CD5, CD7, CD19, TRAC, BCMA, TCRβ, or combinations thereof, or (c) both (a) and (b).

43. The method of claim 42, wherein the solid tumor antigen comprises mesothelin, MR1, guanylate cyclase C (GC-C), epidermal growth factor receptor (EGFR or erbB-1), human epidermal growth factor receptor 2 (HER2 or erbB2), erbB-3, erbB-4, MUC-1, melanoma-associated chondroitin sulfate proteoglycan (MCSP), folate receptor 1 (FOLR1), CD4, CD19, CD20, CD22, CD30, CD33, CD38, CD44, CD44v6, CD44v7/8, CD70, CD123, CD138, CD171, CEA, CSPG4, CXCR5, c-Met, HERV-envelope protein, eriostin, Bigh3, SPARC, BCR, CD79, CD37, EGFRvIII, EGP2, EGP40, IGFr, L1CAM, AXL, Tissue Factor (TF), CD74, EpCAM, EphA2, MRP3cadherin 19 (CDH19), epidermal growth factor 2 (HER2), 5T4, 8H9, αᵥβ₆ integrin, BCMA, B7-H3, B7-H6, CAIX, CA9, FAP, FBP, fetal AchR, FRcc, GD2, GD3, Glypican-1 (GPC1), Glypican-2 (GPC2), Glypican-3 (GPC3), HLA-A1+MAGE1, HLA-A1+NY-ESO-1, IL-13Rcc2, Lewis-Y, KDR, MCSP, Muc1, Muc16, NCAM, NKG2D ligands, NY-ESO-1, PRAME, PSC1, PSCA, PSMA, ROR1, ROR2, SP17, survivin, TAG72, TEMs, carcinoembryonic antigen, HMW-MAA, VEGF, CLDN18.2, or combinations thereof.

44-46. (canceled)

47. The method of claim 1, wherein the CAR-bearing immune cells, transgenic TCR-bearing immune cells, or both are genome edited.

48-57. (canceled)

58. The method of claim 1, wherein the solid tumor is derived from a mesothelioma, cervical cancer, pancreatic cancer, ovarian cancer, squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulvar cancer, thyroid cancer, hepatic carcinoma, anal carcinoma, penile carcinoma head and neck cancer, or any combination thereof.

59-61. (canceled)

62. The method of claim 6, wherein the half-life extending moiety comprises an Fc, albumin, an albumin-binding polypeptide, Pro/Ala/Ser (PAS), a C-terminal peptide (CTP) of the β subunit of human chorionic gonadotropin, polyethylene glycol (PEG), long unstructured hydrophilic sequences of amino acids (XTEN), hydroxyethyl starch (HES), an albumin-binding small molecule, or a combination thereof.

63. The method of claim 10, wherein the half-life extending moiety comprises an Fc, albumin, an albumin-binding polypeptide, Pro/Ala/Ser (PAS), a C-terminal peptide (CTP) of the β subunit of human chorionic gonadotropin, polyethylene glycol (PEG), long unstructured hydrophilic sequences of amino acids (XTEN), hydroxyethyl starch (HES), an albumin-binding small molecule, or a combination thereof.

64. The method of claim 1, wherein the IL-7 protein does not comprise amino acid residues 1-25 of the amino acid sequence set forth in any one of SEQ ID NOs: 1-6.

65. The method of claim 1, wherein the IL-7 protein comprises: (i) amino acid residues 26-178 of SEQ ID NO: 1, (ii) amino acid residues 26-155 of SEQ ID NO: 2, (iii) amino acid residues 26-155 of SEQ ID NO: 3, (iv) amino acid residues 26-178 of SEQ ID NO: 4, (v) amino acid residues 26-177 of SEQ ID NO: 5, or (iv) amino acid residues 26-177 of SEQ ID NO: 6.
